(19) United States
(12) Patent Application Publication
(10) Pub. No.: US 2024/0301370 A1
(43) Pub. Date: Sep. 12, 2024

(54) Ligand Binding Molecules and Uses Thereof

(71) Applicant: VEGENICS PTY LIMITED, South Yarra (AU)

(72) Inventors: Michael Gerometta, Robertson (AU); Timothy Adams, Lower Plenty (AU)

(21) Appl. No.: 18/523,811

(22) Filed: Nov. 29, 2023

Related U.S. Application Data
(63) Continuation of application No. 16/593,750, filed on Oct. 4, 2019, now Pat. No. 11,866,739.
Application No. 16/593,750 is a continuation of application No. 15/639,573, filed on Jun. 30, 2017, now Pat. No. 10,494,617.
Application No. 15/639,573 is a division of application No. 14/768,701, filed on Aug. 18, 2015, now Pat. No. 9,745,558.
(60) Provisional application No. 61/782,376, filed on Mar. 14, 2013.
(60) Provisional application No. 61/765,841, filed on Feb. 18, 2013.

(51) Int. Cl. C12N 9/12; A61K 38/00; A61K 38/45; A61K 39/395; A61K 45/06; A61K 47/64; C07K 14/71; C07K 16/00

(52) U.S. Cl. CPC C12N 9/12; A61K 38/45; A61K 39/395; A61K 45/06; A61K 47/64; C07K 14/71; C07K 16/00; A61K 38/00; C07K 2319/00; C07K 2319/03; C07K 2319/30; C07K 2319/31; C07K 2319/70; C12N 2710/10043; C12Y 207/10001

(57) ABSTRACT

The present invention is directed to ligand binding molecules and uses thereof to modulate angiogenesis and/or lymphangiogenesis.

### CROSS-REFERENCE TO RELATED APPLICATIONS

This application is a continuation of U.S. patent application Ser. No. 16/593,750, filed on Oct. 4, 2019, now U.S. Pat. No. 11,866,739; which is a continuation of U.S. patent application Ser. No. 15/639,573, filed on Jun. 30, 2017, now U.S. Pat. No. 10,494,617; which is a division of U.S. patent application Ser. No. 14/768,701, accorded § 371(c)(1) date of Aug. 18, 2015, now U.S. Pat. No. 9,745,558; which is a U.S. national stage of International Patent Application No. PCT/AU2014/000114, filed Feb. 13, 2014; which claims the benefit of U.S. Provisional Application No. 61/782,376, filed Mar. 14, 2013, and U.S. Provisional Application No. 61/765,841, filed on Feb. 18, 2013.

### FIELD OF THE INVENTION

This invention relates generally to modulation of vessel growth, especially in ophthalmology and oncology.

### INCORPORATION BY REFERENCE OF MATERIALS SUBMITTED ELECTRONICALLY

This application contains, as a separate part of the disclosure, a computer-readable nucleotide/amino acid sequence listing identified as follows: 41,742 byte extensible Markup Language (.xml) file named “47459E_Seqlisting.xml”; created on Sep. 20, 2023, which is incorporated herein by reference in its entirety.

### BACKGROUND

The vascular endothelial growth factor (VEGF) proteins and their receptors (VEGFRs) play important roles in both vasculogenesis, the development of the embryonic vasculature from early differentiating endothelial cells, angiogenesis, the process of forming new blood vessels from pre-existing ones, and lymphangiogenesis, the process of forming new lymph vessels. The platelet derived growth factor (PDGF) proteins and their receptors (PDGFRs) are involved in regulation of cell proliferation, survival and migration of several cell types.

Dysfunction of the endothelial cell regulatory system is a key feature of cancer and various diseases associated with abnormal vasculogenesis, angiogenesis and lymphangiogenesis. Angiogenesis occurs in embryonic development and normal tissue growth, repair, and regeneration, the female reproductive cycle, the establishment and maintenance of pregnancy, the repair of wounds and fractures. In addition to angiogenesis which takes place in the healthy individual, angiogenic events are involved in a number of pathological processes, notably tumor growth and metastasis, and other conditions in which blood vessel proliferation, especially of the microvascular system, is increased, such as diabetic retinopathy, psoriasis and arthropathies. Inhibition of angiogenesis is useful in preventing or alleviating these pathological processes or slowing progression of them.

Although therapies directed to blockade of VEGF/PDGF signaling through their receptors have shown promise for inhibition of angiogenesis and tumor growth, there remains a need for new or improved compounds and therapies for the treatment of such diseases.

### SUMMARY OF THE INVENTION

The present invention relates to novel compositions and methods of use thereof for the inhibition of aberrant angiogenesis, lymphangiogenesis or both, and inhibition of other effects of Vascular Endothelial Growth Factor-C (VEGF-C) and Vascular Endothelial Growth Factor-D (VEGF-D), each of which is able to bind at least one growth factor receptor tyrosine kinase (i.e., VEGFR-2 or VEGFR-3) and stimulate phosphorylation of the same. The compositions of the invention include ligand binding molecules that bind one or both of human VEGF-C and human VEGF-D. In some embodiments, the ligand binding molecule comprises a polypeptide, e.g., a fragment of a growth factor receptor tyrosine kinase extracellular domain (ECD). The fragment may vary from the wildtype sequence in ways that do not eliminate growth factor binding, and the fragment preferably is engineered in ways described herein to improve its properties as a therapeutic for administration to subjects/patients in need.

The invention also provides nucleic acids encoding such ligand binding molecules. The nucleic acids are useful for expressing the polypeptide ligand binding molecules and also useful, in some embodiments, as a therapeutic for achieving expression of the polypeptide ligand binding molecules in vivo, in a biologically active form.

Administration of the compositions comprising a ligand binding molecule described herein (or polynucleotide encoding it) to patients in need thereof inhibits growth factor stimulation of VEGF receptors (e.g., inhibits phosphorylation of the receptors) and thereby inhibits biological responses mediated through the receptors including, but not limited to, VEGFR-mediated angiogenesis, lymphangiogenesis or both.

VEGF-C and D bind with high affinity to, and stimulate phosphorylation of, at least one VEGF receptor (or receptor heterodimer) selected from VEGFR-2 and VEGFR-3. This statement refers to well-known properties of the growth factors toward their cognate receptors, and is not meant as a limiting feature per se of the ligand binding molecules of the invention. However, preferred ligand binding molecules of the invention do more than simply bind their target growth factors. A preferred ligand binding molecule also inhibits the growth factor(s) to which it binds from stimulating phosphorylation of at least one (and preferably all) of the receptor tyrosine kinases to which the growth factor(s) bind. Stimulation of tyrosine phosphorylation is readily measured using in vitro cell-based assays and anti-phosphotyrosine antibodies. Because phosphorylation of the receptor tyrosine kinases is an initial step in a signaling cascade, it is a convenient indicator of whether the ligand binding molecule is capable of inhibiting growth factor-mediated signal transduction that leads to cell migration, cell growth and other responses. A number of other cell-based and in vivo assays can be used to confirm the growth factor neutralizing properties of ligand binding molecules of the invention.

Ligand binding molecules that are “specific” for a particular growth factor are ligand binding molecules that specifically recognize an active form of the growth factor (e.g., a form found circulating in the body). Preferably, the ligand binding molecules specifically bind other forms of the growth factors as well. By way of example, VEGF-C (and VEGF-D) is translated as a prepro-molecule with extensive amino-terminal and carboxy-terminal propeptides that are cleaved to yield a “fully processed” form of VEGF-C (or VEGF-D) that binds and stimulates VEGFR-2 and VEGFR-3. Ligand binding molecules specific for VEGF-C (or VEGF-D) bind to at least the fully processed form of VEGF-C (or VEGF-D), and preferably also bind to partially processed forms and unprocessed forms.

In one aspect, described herein the ligand binding molecule is a purified or isolated ligand binding polypeptide comprising a first amino acid sequence having at least 80%, or at least 85%, or at least 90%, or at least 92%, or at least 95%, or at least 96%, or at least 97%, or at least 98%, or at least 99% identity to the sequence of amino acids defined by positions 47-115 of SEQ ID NO: 2 or positions 25-115 of SEQ ID NO: 2, with the proviso that positions of the polypeptide corresponding to positions 104-106 of SEQ ID NO: 2 are not identical to N-X-S or N-X-T (X representing any amino acid), wherein the polypeptide binds to at least one ligand polypeptide selected from the VEGF or PDGF families of growth factors, such as human VEGF-A (VEGF), VEGF-B, VEGF-C, VEGF-D, PIGF, PDGF-A, PDGF-B, PDGF-C, and PDGF-D. SEQ ID NO: 2 contains an amino acid sequence for human VEGFR-3, with positions 1-24 of SEQ ID NO: 2 corresponding to a putative signal peptide and position 25 onwards of SEQ ID NO: 2 corresponding to a putative mature form of the receptor lacking a putative signal peptide. The foregoing segments of SEQ ID NO: 2 roughly correspond to or include the first immunoglobulin-like domain of the ECD of human VEGFR-3 (“D1 of VEGFR-3”). Constructs that comprise additional Ig-like domains of VEGFR-3 or other receptors, attached in a manner that result in a ligand binding polypeptide, are specifically contemplated, and constructs that bind different ligands are constructed by varying the receptor components used to make the ligand binding polypeptide. In some variations, the ligand binding polypeptide is based primarily on the extracellular domain of VEGFR-3, and in other embodiments, the ligand binding polypeptide is based on a fusion of segments of other receptor tyrosine kinases, such as VEGFR-1 and/or VEGFR-2 and/or PDGFR-α and/or PDGFR-β. In embodiments based primarily on VEGFR-3, the at least one ligand is a natural ligand for VEGFR-3, such as a VEGF-C or a VEGF-D polypeptide.

In some embodiments, the ligand binding polypeptide comprises a second amino acid sequence at least 80%, or at least 85%, or at least 90%, or at least 92%, or at least 95%, or at least 96%, or at least 97%, or at least 98%, or at least 99% identical to the sequence of amino acids defined by positions 154-210 of SEQ ID NO:2 or positions 248-314 of SEQ ID NO:2, wherein the N-terminal residue of the second amino acid sequence is connected to the C-terminal residue of the first amino acid sequence either directly or via a spacer, wherein the polypeptide binds to at least one ligand polypeptide selected from the VEGF or PDGF families of growth factors, such as human VEGF-A (VEGF), VEGF-B, VEGF-C, VEGF-D, PIGF, PDGF-A, PDGF-B, PDGF-C, and PDGF-D. The sequence of amino acids defined by positions of the polypeptide corresponding to positions 154-210 roughly corresponds to or includes the second immunoglobulin-like domain of the ECD of human VEGFR-3 (“D2 of VEGFR-3”). The sequence of amino acids defined by positions of the polypeptide corresponding to positions 248-314 roughly corresponds to or includes the third immunoglobulin-like domain of the ECD of human VEGFR-3 (“D3 of VEGFR-3”). Where the second amino acid sequence comprises a sequence of amino acids roughly corresponding to or including D2 of VEGFR-3, it is preferred that the ligand binding polypeptide comprises a third amino acid sequence at least 80%, or at least 85%, or at least 90%, or at least 92%, or at least 95%, or at least 96%, or at least 97%, or at least 98%, or at least 99% identical to the sequence of amino acids defined by positions 248-314 of SEQ ID NO:2, wherein the N-terminal residue of the third amino acid sequence is connected to the C-terminal residue of the second amino acid sequence either directly or via a spacer, wherein the polypeptide binds to at least one ligand polypeptide selected from the VEGF or PDGF families of growth factors, such as human VEGF-A (VEGF), VEGF-B, VEGF-C, VEGF-D, PIGF, PDGF-A, PDGF-B, PDGF-C, and PDGF-D. In other words, in embodiments where the ligand binding polypeptide comprises amino acid sequences roughly corresponding to or including the D1 and D2 of VEGFR-3, it is preferred that the ligand binding polypeptide also comprises an amino acid sequence roughly corresponding to or including the D3 of VEGFR-3.

In embodiments where the ligand binding polypeptide comprises amino acid sequences roughly corresponding to two or more component domains of VEGFR-3, the component domains may be connected directly to each other or may be connected via one or more spacers. Preferably, the component domains are connected by one or more spacers. In one embodiment, the spacer comprises one or more peptide sequences between the component domains which is (are) between 1-100 amino acids, preferably 1-50 amino acids in length. In one embodiment, the spacer between two component domains substantially consists of peptide sequences naturally connected to the component domain in native VEGFR-3.

In embodiments where the ligand binding polypeptide comprises amino acid sequences roughly corresponding to or including contiguous component domains of VEGFR-3 (for example, D1-D2 or D1-D2-D3), the component domains are connected via one or more spacers comprising one or more peptide sequences between the component domains which is (are) between 1-100 amino acids, preferably 1-50 amino acids in length. In one embodiment, the spacer between two component domains substantially consists of peptide sequences corresponding to those connecting the respective contiguous component domains in the native VEGFR-3. In some embodiments, the spacer between two contiguous component domains comprises an amino acid sequence at least 80%, or at least 85%, or at least 90%, or at least 92%, or at least 95%, or at least 96%, or at least 97%, or at least 98%, or at least 99% identical to the sequence of amino acids that connects the contiguous domains in the native VEGFR-3.

In one embodiment, where the ligand binding polypeptide comprises amino acid sequences roughly corresponding to or including the D1 and D2 of VEGFR-3, the component domains D1 and D2 are connected via a spacer amino acid sequence having at least 80%, or at least 85%, or at least 90%, or at least 92%, or at least 95%, or at least 96%, or at least 97%, or at least 98%, or at least 99% identity to the sequence of amino acids defined by positions 116-153 of SEQ ID NO: 2. Where the ligand binding polypeptide comprises amino acid sequences roughly corresponding to or including the D1, D2 and D3 of VEGFR-3, the component domains D2 and D3 are connected via a spacer amino acid sequence having at least 80%, or at least 85%, or at least 90%, or at least 92%, or at least 95%, or at least 96%, or at least 97%, or at least 98%, or at least 99% identity to the sequence of amino acids defined by positions 211-247 of SEQ ID NO: 2.

In some embodiments, the purified or isolated ligand binding polypeptide comprises an amino acid sequence at least 80%, or at least 85%, or at least 90%, or at least 92%, or at least 95%, or at least 96%, or at least 97%, or at least 98%, or at least 99% identical to the sequence of amino acids defined by positions 47-210 of SEQ ID NO: 2, or positions 25-210 of SEQ ID NO: 2, or positions 47-314 of SEQ ID NO: 2, or positions 25-314 of SEQ ID NO: 2, or positions 47-752 or 47-775 of SEQ ID NO: 2, or positions 25-752 or 25-775 of SEQ ID NO: 2, with the proviso that positions of the polypeptide corresponding to positions 104-106 of SEQ ID NO: 2 are not identical to N-X-S or N-X-T, wherein the polypeptide binds to at least one ligand polypeptide selected from human VEGF-A, VEGF-C, VEGF-C, VEGF-D and PIGF. In one variation, the amino acid corresponding to position 104 of SEQ ID NO: 2 is deleted and replaced with another amino acid (such as glutamine, aspartate, glutamate, arginine and lysine). Positions 47-210 include the first two immunoglobulin-like domains of the human VEGFR-3 ECD, as well as VEGFR-3 ECD sequence between the first two Ig-like motifs. Positions 47-314 include the first three immunoglobulin-like domains of the human VEGFR-3 ECD, as well as VEGFR-3 ECD sequence between these Ig-like motifs.

More generally, a ligand binding polypeptide of the invention comprises an amino acid sequence at least 80%, or at least 85%, or at least 90%, or at least 92%, or at least 95%, or at least 96%, or at least 97%, or at least 98%, or at least 99% identical to a fragment of the VEGFR-3 amino acid sequence set forth in SEQ ID NO: 2, wherein the amino terminus of the fragment is any amino acid selected from positions 25, 26, 27, 28, 29, 30, 31, 32, 33, 34, 35, 36, 37, 38, 39, 40, 41, 42, 43, 44, 45, 46, 47, 48, 49, and 50 of SEQ ID NO: 2; and wherein the carboxy terminus of the fragment is any amino acid selected from positions 110-775 of SEQ ID NO: 2 (e.g., positions 110, 111, 112, 113, 114, 115, 116, 747, 748, 749, 750, 751, 752, 753, 754, 755, 756, 757, 758, 759, 760, 761, 762 ,763, 764, 765, 766, 767, 768, 769, 770, 771, 772, 773, 774, 775), with the proviso that positions of the polypeptide corresponding to positions 104-106 of SEQ ID NO: 2 are not identical to N-X-S or N-X-T. For reasons that will be readily apparent from the description herein, the variation permitted is not variation that introduces new glycosylation sequons that are not found in wildtype VEGFR-3.

In another aspect, described herein the ligand binding molecule is a purified or isolated ligand binding polypeptide that comprises an amino acid sequence that is identical to the sequence of amino acids defined by positions of the polypeptide corresponding to positions 47-115 of SEQ ID NO: 2, 47-210 of SEQ ID NO: 2, 47-314 of SEQ ID NO: 2, 47-752 or 47-775 of SEQ ID NO: 2, or 25-752 or 25-775 of SEQ ID NO: 2, with the proviso that positions of the polypeptide corresponding to positions 104-106 of SEQ ID NO: 2 are not identical to N-X-S or N-X-T. In one variation, the amino acid corresponding to position 104 of SEQ ID NO: 2 is deleted and replaced with another amino acid (such as glutamine, aspartate, glutamate, arginine and lysine).

In another aspect, described herein the ligand binding molecule is a purified or isolated ligand binding polypeptide comprising an amino acid sequence having at least 80%, or at least 85%, or at least 90%, or at least 92%, or at least 95%, or at least 96%, or at least 97%, or at least 98%, or at least 99% identity to the sequence of amino acids defined by positions 47-115 of SEQ ID NO: 2, wherein positions of the polypeptide corresponding to positions 104-106 of SEQ ID NO: 2 are a putative VEGFR-3 glycosylation sequon, and wherein said putative glycosylation sequon is eliminated from the amino acid sequence of the ligand binding polypeptide. The term “eliminated” as used in this context means an alteration of the primary amino acid sequence in at least one position (by substitution, deletion or insertion) to destroy the N-X-T sequon motif.

The invention also includes multimeric ligand binding constructs comprising two or more ligand binding molecules as described herein, covalently or non-covalently attached to each other to form a dimeric or multimeric structure. In some variations, the attachment occurs between the VEGFR-3-like sequences of the ligand binding polypeptides; in other variations, the attachment occurs between heterologous polypeptides attached to one or both of the VEGFR-3 like sequences.

Reference herein to a ligand binding molecule or ligand binding polypeptide described herein includes reference to variants thereof as defined above with the proviso that such ligand binding polypeptides or molecules (whether monomeric, dimeric or a higher multimer) contains at least an Ig-like motif similar or identical to Ig-like motif 1 of VEGFR-3 (e.g., about 47-115 of SEQ ID NO: 2), with the proviso that positions of the polypeptide corresponding to positions 104-106 of SEQ ID NO: 2 (which represent an N-linked glycosylation sequon in the native VEGFR-3 sequence) are not identical to N-X-S or N-X-T.

In another aspect, described herein is a ligand binding molecule which is an isolated or purified ligand binding polypeptide comprising the first immunoglobulin-like domain of a VEGFR-3ΔN2 polypeptide. As used herein, the term “VEGFR-3ΔN2 polypeptide” refers to a polypeptide having at least 95% identity to the sequence of amino acids defining the ECD of human VEGFR-3, with the proviso that the portion of the polypeptide's sequence corresponding to the second putative glycosylation sequon, NDT, is mutated such that it no longer fits the N-X-S/T SEQUON motif, e.g., due to substitution at one of the positions. In some embodiments, the purified polypeptide comprises the first two immunoglobulin-like domains of the VEGFR-3ΔN2 polypeptide, and preferably includes the VEGFR-3 sequence between those domains. In some embodiments, the purified polypeptide comprises the first thee immunoglobulin-like domains of the VEGFR-3ΔN2 polypeptide, and preferably includes the VEGFR-3 sequence between those domains.

In yet another aspect, described herein is a ligand binding molecule which is a polypeptide comprising an ECD fragment of human VEGFR-3, fused to a fusion partner wherein the amino acid sequence of the ECD fragment of VEGFR-3 is modified from wildtype VEGFR-3 to eliminate the second putative N-linked glycosylation sequon of wildtype VEGFR-3, wherein the polypeptide is soluble in human serum and binds human VEGF-C or human VEGF-D; and wherein the fusion partner improves solubility or serum half-life of the ECD fragment (e.g., compared to an identical fragment that is not fused to a fusion partner). In some embodiments, the fusion partner is a heterologous polypeptide.

In some embodiments, the ligand binding polypeptide or ligand binding molecule binds human VEGF-C or human VEGF-D. In some embodiments, the ligand binding polypeptide or ligand binding molecule inhibits VEGF-C-or VEGF-D-binding to VEGFR-3 or inhibits VEGF-C- or VEGF-D-mediated stimulation of VEGFR-3 in a cell expressing VEGFR-3 on its surface. The inhibition of stimulation can be demonstrated, for example, by measuring receptor phosphorylation, or by measuring cellular growth in vitro or in vivo, or by measuring vessel growth or other tissue-level changes in vivo.

The ligand binding molecule preferably binds human VEGF-C with a K_{d} of about 1nM or less (e.g., 500 pM, 400 pM, 300 pM, 200 pM, 100 pM, 50 pM, 10 pM or less). The ligand binding molecule preferably binds human VEGF-D with a K_{d} of about 5 nM or less (e.g., 2 nM, 1 nM, 500 pM, 400 pM, 300 pM, 200 pM, 100 pM, 50 pM, 10 pM or less).

In another aspect, the purified or isolated ligand binding molecule comprises amino acids 22-290 of SEQ ID NO: 3, amino acids 23-290 of SEQ ID NO: 3, amino acids 23-537 of SEQ ID NO: 3 or amino acids 22-537 of SEQ ID NO: 3. In still other variations, the molecule comprises an amino acid sequence at least 80%, or at least 85%, or at least 90%, or at least 92%, or at least 95%, or at least 96%, or at least 97%, or at least 98%, or at least 99% identical to either of the foregoing sequences, with the proviso that the sequence of the polypeptide that corresponds to (aligns with) the VEGFR-3 N2 sequon is not a glycosylation sequence.

As described herein, ligand binding molecules can be chemically modified (e.g., glycosylation, pegylation, etc.) to impart desired characteristics, while maintaining their specific growth factor binding properties. Ig-like domains I-III of VEGFR-3 comprises five putative N-glycosylation sites (referred to herein as N1, N2, N3, N4 and N5 sequons of VEGFR-3, respectively). N1 corresponds to amino acids 33-35 of SEQ ID NO: 2; N2 corresponds to amino acids 104-106 of SEQ ID NO: 2; N3 corresponds to amino acids 166-168 of SEQ ID NO: 2; N4 corresponds to amino acids 251-253 of SEQ ID NO: 2 and N5 corresponds to amino acids 299-301 of SEQ ID NO: 2. In some embodiments, a ligand binding molecule described herein comprises a modification in the N2 sequon of the molecule. For example, in some embodiments, the amino acid in the ligand binding molecule corresponding to position 104 of SEQ ID NO: 2 is deleted and replaced with another amino acid. Conservative substitutions are preferred. In some embodiments, the amino acid corresponding to position 104 of SEQ ID NO: 2 is deleted and replaced with an amino acid selected from the group consisting of glutamine, aspartate, glutamate, arginine and lysine. In embodiments where the N2 sequon of SEQ ID NO: 2 is modified as described above, the N1, N3, N4 and N5 sequons of SEQ ID NO: 2 are preferably unaltered in terms of amino acid sequence.

As described herein, ligand binding molecules can be connected to a fusion partner either directly or via a linker. A fusion partner may be any heterologous component that enhances the functionality of the ligand binding molecule. An exemplary peptide fusion partner comprises an immunoglobulin constant domain (Fc) fragment. In some embodiments, the immunoglobulin constant fragment comprises an amino acid sequence having at least 80%, or at least 85%, or at least 90%, or at least 92%, or at least 95%, or at least 96%, or at least 97%, or at least 98%, or at least 99% identity, or having 100% identity to amino acids 306-537 of SEQ ID NO: 3.

As described herein, ligand binding molecules can be chemically modified to, for example, facilitate connection to a fusion partner (such as, for example, a heterologous peptide) or impart desired characteristics (such as, for example, increase the serum half-life, increase the solubility in an aqueous medium and enable targeting to a specific cell population, e.g., tumor cells or retinal cells).

In some embodiments, a ligand binding molecule described herein optionally comprises at least one PEG moiety attached to the molecule. For example, in some embodiments, PEG of about 20-40 kDa is attached to the amino terminus of the ligand binding molecule.

In some embodiments, a ligand binding molecule as described herein optionally comprises a linker connecting the fusion partner, such as, for example, a heterologous peptide to the ligand binding polypeptide, such as the factor Xa linker sequence PIEGRGGGGG (SEQ ID NO: 4).

In other embodiments, the ligand binding molecule comprises a polypeptide in which a C-terminal amino acid of the ligand binding polypeptide is directly attached to an N-terminal amino acid of the heterologous peptide fusion partner by a peptide bond. In some embodiments, the ligand binding polypeptide and the heterologous peptide are attached (directly or through a linker polypeptide) by amide bonding to form a single polypeptide chain.

In some variations, the ligand binding molecule comprises a signal peptide that directs secretion of the molecule from a cell that expresses the molecule.

Nucleic acids (polynucleotides) of the invention include nucleic acids that encode polypeptide ligand binding molecules, which may be used for such applications as gene therapy and recombinant in vitro expression of polypeptide ligand binding molecules. In some embodiments, nucleic acids are purified or isolated. In some embodiments, polynucleotides further comprise a promoter sequence operatively connected to a nucleotide sequence encoding a polypeptide, wherein the promoter sequence promotes transcription of the sequence that encodes the polypeptide in a host cell. Polynucleotides may also comprise a polyadenylation signal sequence. In some variations, the nucleic acid has a coding nucleotide sequence similar to a wild type human VEGFR-3-encoding nucleic acid. For example, the nucleic acid comprises a coding nucleotide sequence having at least 80%, or at least 85%, or at least 90%, or at least 92%, or at least 95%, or at least 96%, or at least 97%, or at least 98%, or at least 99% identity to the human VEGFR-3 sequence set forth in SEQ ID NO: 1, or to a fragment thereof. By way of example, in the context of a nucleotide sequence encoding amino acids 47-314 of SEQ ID NO: 2, modified at the N2 sequon, an exemplary nucleic acid comprises a coding nucleotide sequence having at least 80%, or at least 85%, or at least 90%, or at least 92%, or at least 95%, or at least 96%, or at least 97%, or at least 98%, or at least 99% identity to the human VEGFR-3 sequence set forth in positions 157 to 961 of SEQ ID NO: 1, which correspond to codons 47-314.

Vectors comprising polynucleotides are also aspects of the invention. Such vectors may comprise an expression control sequence operatively connected to the sequence that encodes the polypeptide. In some variations, the vector is selected to optimize in vitro recombinant expression in a chosen host cell, such as a eukaryotic host cell. In some variations, the vector is selected for in vivo delivery. For example, the vector may be selected from the group consisting of a lentivirus vector, an adeno-associated viral vector, an adenoviral vector, a liposomal vector, and combinations thereof. In some embodiments, the vector comprises a replication-deficient adenovirus, said adenovirus comprising the polynucleotide operatively connected to a promoter and flanked by adenoviral polynucleotide sequences.

Host cells comprising the polynucleotides, vectors and other nucleic acids, and methods for using the same to express and isolate the ligand binding molecules are also aspects of the invention. Eukaryotic host cells, including Chinese Hamster Ovary (CHO) cells and other mammalian cell lines comprising a polynucleotide encoding a ligand binding polypeptide or ligand binding molecule described herein are specifically contemplated. In some variations, the cell line is selected or engineered to introduce a human or human-like glycosylation at glycosylation sequons of polypeptides produced in the cells.

Methods of making a ligand binding polypeptide or molecule described herein are also contemplated. (Such methods could also be described as uses of the polynucleotides or cells of the invention.) In one aspect, the method comprises growing a cell that has been transformed or transfected with a polynucleotide or vector described herein under conditions in which the ligand binding polypeptide or ligand binding molecule encoded by the polynucleotide is expressed. In some embodiments, the method further comprises purifying or isolating the ligand binding polypeptide or the ligand binding molecule from the cell or from a growth media of the cell. In some embodiments, the method further includes attaching one or more polyethylene glycol (PEG) or other moieties to the expressed and purified/isolated polypeptide.

The invention also includes compositions comprising a polypeptide, ligand binding molecule or nucleic acid encoding the same, together with a pharmaceutically acceptable diluent, adjuvant, or carrier medium. In some embodiments, the composition is formulated for local administration to the eye (e.g., a topical formulation such as an ointment or eyedrop, or a formulation suitable for intravitreal injection). In other embodiments, the composition is formulated for local administration to a tumor or to the organ or tissue from which the tumor has been surgically removed, e.g., by intravenous injection or injection directly into the affected tissue, or application by way of device during tumor resection.

The invention also includes methods of using materials described herein (polypeptides, molecules and constructs, polynucleotides and vectors, transformed cells, compositions) for inhibiting vessel growth (blood vessel and/or lymphatic vessel) in therapeutic and prophylactic contexts. Methods of using as described herein can alternatively be characterized as uses of the various materials for the stated indication. Exemplary subjects for treatment include humans and other primates, livestock (e.g., bovines, equines, porcines), zoo animals (e.g., felines, canines, pachyderms, cervidae), and pets (e.g., dogs, cats), and rodents.

In some variations, the invention includes a method of inhibiting neovascularization in a subject, the method comprising administering to the subject any of the foregoing materials or compositions, in an amount effective to inhibit neovascularization in the subject. Exemplary pathogenic neovascular conditions include those of the eye, and tumor neovascularization.

In some variations, the invention includes a method of inhibiting retinal neovascularization in a subject, the method comprising administering to the subject materials or compositions as described herein, in an amount effective to inhibit retinal neovascularization in the subject. In related variations, the invention includes a method of treating a subject having an ocular disorder associated with retinal neovascularization, the method comprising administering to the subject a material or composition as described herein and summarized above, in an amount effective to inhibit retinal neovascularization in the subject. For example, a composition as described herein is administered locally to the eye of the subject, such as by eye drops or other topical administration, by subconjunvtival administration (e.g., injection), by intravitreal injection, or by intravitreal implant.

Compositions preferably are administered in an amount and at a repeated dosing frequency and duration effective to inhibit VEGF-C and/or VEGF-D in the eye of the subject from binding to or stimulating VEGFR-2 and/or VEGFR-3 expressed in cells of the eye or vessels of the eye. Such beneficial effect may be measured in terms of slowing or halting of deterioration/progression in the pathological eye condition (such as macular degeneration, diabetic retinopathy and macular telangiectasia), or improvement in clinical symptoms. The beneficial effect also may be observable in terms of monitoring of vessel growth in and around the targeted tissue.

Methods and uses described herein may be practiced in combination with additional therapeutic agents or treatments (e.g., forms of radiation), as described herein in detail.

Methods (or uses) of the invention described herein may be carried out with one or more ligand binding molecule, or with at least one ligand binding molecule in combination with another therapeutic (such as a standard of care therapeutic for the treatment of cancer or for the treatment of a back of the eye disorder). In embodiments wherein the ligand binding molecules are for the treatment of a back of the eye disorder, contemplated additional therapies include focal laser treatment (or photocoagulation), scatter laser treatment (or panretinal photocoagulation) and virectomy. In some embodiments, antibiotics are also administered to the subject receiving treatment.

In embodiments where the ligand binding molecules described herein are for use in the treatment of cancer, contemplated standard of care therapeutics include anti-sense RNA, RNA interference, bispecific antibodies, other antibody types, and small molecules, e.g., chemotherapeutic agents, which target growth factors and/or their receptors. A cytokine, radiotherapeutic agent, or radiation therapy may also be used in combination with a ligand binding molecule described herein. The chemotherapeutic agent or radiotherapeutic agent may be a member of the class of agents including an anti-metabolite; a DNA-damaging agent; a cytokine or growth factor; a covalent DNA-binding drug; a topoisomerase inhibitor; an anti-mitotic agent; an anti-tumor antibiotic; a differentiation agent; an alkylating agent; a methylating agent; a hormone or hormone antagonist; a nitrogen mustard; a radiosensitizer; and a photosensitizer. Specific examples of these agents are described elsewhere in the application. Combination therapies are preferably synergistic, but they need not be, and additive therapies are also considered aspects of the invention.

In addition to their use in methods, the ligand binding molecules may be combined or packaged with other therapeutics in kits or as unit doses. Neoplastic diseases are not the only diseases that may be treated with the ligand binding molecules. The ligand binding molecules may be used as therapeutics for any disease associated with aberrant angiogenesis or lymphangiogenesis.

The invention can also described in the following additional embodiments:
48. A purified or isolated ligand binding polypeptide comprising an amino acid sequence having at least 95% identity to the sequence of amino acids defined by positions 47-115 of SEQ ID NO: 2, with the proviso that positions of the polypeptide corresponding to positions 104-106 of SEQ ID NO: 2 are not identical to N-X-S or N-X-T, wherein the polypeptide binds to at least one ligand polypeptide selected from human VEGF-C, VEGF-D, and PIGF.
49. The purified or isolated ligand binding polypeptide according to paragraph 48, comprising an amino acid sequence having at least 95% identity to the sequence of amino acids defined by positions 47-210 of SEQ ID NO: 2, with the proviso that positions of the polypeptide corresponding to positions 104-106 of SEQ ID NO: 2 are not identical to N-X-S or N-X-T.
50. The purified or isolated ligand binding polypeptide according to paragraph 48, comprising an amino acid sequence having at least 95% identity to the sequence of amino acids defined by positions 47-314 of SEQ ID NO: 2, with the proviso that positions of the polypeptide corresponding to positions 104-106 of SEQ ID NO: 2 are not identical to N-X-S or N-X-T.
51. The purified or isolated ligand binding polypeptide according to paragraph 48, comprising an amino acid sequence having at least 95% identity to the sequence of amino acids defined by positions 47-752 of SEQ ID NO: 2, with the proviso that positions of the polypeptide corresponding to positions 104-106 of SEQ ID NO: 2 are not identical to N-X-S or N-X-T.
52. The purified or isolated ligand binding polypeptide according to any one of paragraphs 48-51 that retains four N-glycosylation sequon sites corresponding to positions 33-35 of SEQ ID NO: 2, positions 166-168 of SEQ ID NO: 2, positions 251-253 of SEQ ID NO: 2, and positions 299-301 of SEQ ID NO: 2.
53. The purified or isolated ligand binding polypeptide according to paragraph 52, that is glycosylated at said four N-glycosylation sequon sites.
54. The purified or isolated ligand binding polypeptide according to any one of paragraphs 48-53 that is a soluble polypeptide.
55. The purified or isolated ligand binding polypeptide according to any one of paragraphs 48-54, comprising an amino acid sequence that is identical to the sequence of amino acids defined by positions 47-115 of SEQ ID NO: 2, positions 47-210 of SEQ ID NO: 2 , positions 47-314 of SEQ ID NO: 2, or positions 47-752 of SEQ ID NO: 2, with the proviso that positions of the polypeptide corresponding to positions 104-106 of SEQ ID NO: 2 are not identical to N-X-S or N-X-T.
56. The purified or isolated ligand binding polypeptide according to any one of paragraphs 48-55 that binds human VEGF-C or human VEGF-D.
57. The purified or isolated ligand binding polypeptide according to paragraph 56, that inhibits VEGF-C-or VEGF-D-binding to VEGFR-3 or inhibits VEGF-C-or VEGF-D-mediated stimulation of VEGFR-3 in a cell expressing VEGFR-3 on its surface.
58. The purified or isolated ligand binding polypeptide according to any one of paragraphs 48-57 that binds human VEGF-C with a Kd or 1 nM or less.
59. The purified or isolated ligand binding polypeptide according to any one of paragraphs 48-57, that binds human VEGF-D with a Kd of 5 nM or less.
60. The purified or isolated ligand binding polypeptide according to any one of paragraphs 48-59, wherein the amino acid in the polypeptide corresponding to position 104 of SEQ ID NO: 2 is deleted or replaced with another amino acid.
61. The purified or isolated ligand binding polypeptide according to paragraph 55, wherein the amino acid at position 104 of SEQ ID NO: 2 is deleted or replaced with another amino acid selected from the group consisting of glutamine, aspartate, glutamate, arginine and lysine.
62. The purified or isolated ligand polypeptide according to any one of paragraphs 48-56, wherein the polypeptide comprises amino acids 23-290 of SEQ ID NO: 3.
63. The purified or isolated ligand binding polypeptide according to any one of paragraphs 48-62, further comprising a signal peptide.
64. The purified or isolated ligand binding polypeptide according to any one of paragraphs 48-63, further comprising at least one polyethylene glycol moiety attached to the polypeptide.
65. The purified or isolated ligand binding polypeptide according to paragraph 64, comprising polyethylene glycol of about 20-40 kDa attached to the amino terminus of the polypeptide.
66. A ligand binding molecule comprising the ligand binding polypeptide according to any one of paragraphs 48-65 connected to a heterologous peptide.
67. The ligand binding molecule according to paragraph 66, wherein the heterologous peptide comprises an immunoglobulin constant domain fragment.
68. The ligand binding molecule according to paragraph 66, wherein the immunoglobulin constant domain fragment is an IgG constant domain fragment.
69. The ligand binding molecule according to paragraph 67, wherein the immunoglobulin constant fragment comprises amino acids 306-537 of SEQ ID NO: 3.
70. The ligand binding molecule according to paragraph 66, wherein the ligand binding molecule comprises amino acids 22-537 of SEQ ID NO: 3.
71. The ligand binding molecule according to any one of paragraphs 66-70, optionally comprising a linker connecting the heterologous peptide to the ligand binding polypeptide.
72. The ligand binding molecule according to any one of paragraphs 66-70 that comprises a polypeptide in which a C-terminal amino acid of the ligand binding polypeptide is directly attached to an N-terminal amino acid of the heterologous peptide by a peptide bond.
73. The ligand binding molecule according to any one of paragraphs 66-72, further comprising a signal peptide that directs secretion of the molecule from a cell that expresses the molecule.
74. The ligand binding molecule according to paragraph 66, wherein the molecule comprises the amino acid sequence set forth in SEQ ID NO: 3.
75. The ligand binding molecule according to any one of paragraphs 66-70, wherein the ligand binding polypeptide and the heterologous peptide are linked by amide bonding to form a single polypeptide chain.
76. The ligand binding polypeptide according to any one of paragraphs 48-65 or the ligand binding molecule according to any one of paragraphs 66-75, further comprising a detectable label.
77. A conjugate comprising the ligand binding polypeptide according to any one of paragraphs 48-65 or the ligand binding molecule according to any one of paragraphs 66-75 and a chemotherapeutic agent.
78. An isolated polynucleotide comprising a coding nucleotide sequence encoding the ligand binding polypeptide according to any one of paragraphs 48-65 or the ligand binding molecule according to any one of paragraphs 66-75.
79. The polynucleotide according to paragraph 78, further comprising a promoter sequence operatively connected to the coding nucleotide sequence to promote transcription of the coding nucleotide sequence in a host cell.
80. A vector comprising the polynucleotide of paragraph 78 or paragraph 79.
81. The vector according to paragraph 80, further comprising an expression control sequence operatively connected to the coding nucleotide sequence.
82. The vector according to paragraph 80, wherein said vector is selected from the group consisting of a lentivirus vector, an adeno-associated viral vector, an adenoviral vector, a liposomal vector, and combinations thereof.
83. The vector according to paragraph 80, wherein said vector comprises a replication-deficient adenovirus, said adenovirus comprising the polynucleotide operatively connected to a promoter and flanked by adenoviral polynucleotide sequences.
84. An isolated cell or cell line transformed or transfected with a polynucleotide according to paragraph 78 or 79 or with a vector according to paragraph 80-83.
85. The isolated cell or cell line according to paragraph 84 that is a eukaryotic cell.
86. The isolated cell or cell line according to paragraph 84 that is a human cell.
87. The isolated cell or cell line according to paragraph 84, that is a Chinese Hamster Ovary (CHO) cell.
88. A method of making a ligand binding polypeptide comprising growing a cell according to any one of paragraphs 84-87 under conditions in which the ligand binding polypeptide or ligand binding molecule encoded by the polynucleotide is expressed.
89. The method according to paragraph 88, further comprising purifying or isolating the ligand binding polypeptide or the ligand binding molecule from the cell or from a growth media of the cell.
90. A composition comprising a purified ligand binding polypeptide or ligand binding molecule according to any one of paragraphs 48-76 and a pharmaceutically acceptable diluent, adjuvant, excipient, or carrier.
91. A composition comprising a polynucleotide or vector according to any one of paragraphs 78-83 and a pharmaceutically acceptable diluent, adjuvant, excipient, or carrier.
92. The composition according to paragraph 90 or paragraph 91, that is formulated for topical administration.
93. The composition according to paragraph 92, that is in the form of a solid, a paste, an ointment, a gel, a liquid, an aerosol, a mist, a polymer, a film, an emulsion, or a suspension.
94. The composition according to paragraph 90 or paragraph 91, that is formulated for intravitreal administration.
95. A method of inhibiting neovascularization in a subject, the method comprising administering to the subject a composition according to any one of paragraphs 90-94 in an amount effective to inhibit neovascularization in the subject.
96. A method of inhibiting retinal neovascularization in a subject, the method comprising administering to the subject a composition according to any one of paragraphs 90-94, in an amount effective to inhibit retinal neovascularization in the subject.
97. A method of treating a subject having an ocular disorder associated with retinal neovascularization, the method comprising administering to the subject a composition according to any one of paragraphs 90-95, in an amount effective to inhibit retinal neovascularization in the subject.
98. Use of a composition according to any one of paragraphs 90-94 for inhibiting neovascularization, such as retinal neovascularization or tumor neovascularization, in a subject in need thereof.
99. The method or use according to any one of paragraphs 96-98, wherein the composition is administered locally to the eye of the subject.
100. The method or use according to paragraph 99, wherein the composition is administered by intravitreal injection.
101. The method or use according to paragraph 99, wherein the composition is administered by topical administration.
102. The method or use according to any one of paragraphs 96-101, wherein the composition is administered in an amount effective to inhibit VEGF-C and/or VEGF-D in the eye of the subject from binding to or stimulating VEGFR-2 and/or VEGFR-3 expressed in cells of the eye or vessels of the eye.
103. The method or use of paragraph 97 or 98, wherein the ocular disorder is selected from the group consisting of macular degeneration, diabetic retinopathy and macular telangiectasia.
104. The method or use according to any one of paragraphs 96-103, further comprising administering an antibiotic to the subject.
105. The method according to paragraph 104, wherein the antibiotic is selected from the group consisting of amikacin, gentamicin, kanamycin, neomycin, netilmicin, streptomycin, tobramycin, teicoplanin, vancomycin, azithromycin, clarithromycin, clarithromycin, dirithromycin, erythromycin, roxithromycin, troleandomycin, amoxicillin, ampicillin, azlocillin, carbenicillin, cloxacillin, dicloxacillin, flucloxacillin, meziocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, mafenide, sulfacetamide, sulfamethizole, sulfasalazine, sulfisoxazole, trimethoprim, cotrimoxazole, demeclocycline, doxycycline, minocycline, oxytetracycline, and tetracycline.
106. The method or use according to paragraph 95-98, wherein the subject has been diagnosed with a tumor, and wherein the composition is administered in an amount effective to inhibit neovascularization in the tumor.
107. The method or use according to paragraph 106, wherein the composition is administered locally to the tumor or to the organ or tissue from which the tumor has been surgically removed.
108. The method or use according to paragraph 106, wherein the composition is administered in an amount effective to inhibit VEGF-C and/or VEGF-D in the tumor of the subject from binding to or stimulating VEGFR-2 and/or VEGFR-3 expressed in tumor cells.

This summary of the invention is not intended to be limiting or comprehensive, and additional embodiments are described in the drawings and detailed description, including the examples. All such embodiments are aspects of the invention. Moreover, for the sake of brevity, various details that are applicable to multiple embodiments have not been repeated for every embodiment. Variations reflecting combinations and rearrangements of the embodiments described herein are intended as aspects of the invention. In addition to the foregoing, the invention includes, as an additional aspect, all embodiments of the invention narrower in scope in any way than the variations specifically mentioned above. For example, for aspects described as a genus or range, every subgenus, subrange or species is specifically contemplated as an embodiment of the invention.

### BRIEF DESCRIPTION OF THE FIGURE

FIG. 1A shows the PK profiles of VGX-300 and VGX-301-ΔN2 produced by transient CHO expression. FIG. 1B shows the PK profiles of VGX-300 and VGX-301-ΔN2 produced by transient HEK expression.

FIG. 2A and FIG. 2B demonstrate that both VGX-300 and VGX-301-ΔN2 specifically bound to both VEGF-C and VEGF-D.

FIG. 3A and FIG. 3B show VGX-300 blocks VEGF-C and VEGF-D binding and cross-linking of VEGFR-2 (FIG. 3A) and VEGFR-3 (FIG. 3B).

FIG. 4A and FIG. 4B show VGX-300 and VGX-300-N2 block VEGF-C (FIG. 4A) and VEGF-D (FIG. 4B) binding and cross-linking of VEGFR-3 in a cell-based Ba/F3 assay. Data points represent the average of n≥2±SD.

FIG. 5A-FIG. 5E show the pharmacokinetics and ocular biodistribution in rabbits following intravitreal administration.

### DETAILED DESCRIPTION

The present invention is based in part on research demonstrating that fragments of the ECD of human VEGFR-3 having one or modifications in an N-glycan region of the ECD are capable of binding to and neutralizing human VEGF-C and human VEGF-D in vitro and are also capable of inhibiting vessel development in animal models of age-related macular degeneration.

Growth factor receptor tyrosine kinases generally comprise three principal domains: an extracellular domain (ECD), a transmembrane domain, and an intracellular domain. The ECD binds ligands, the transmembrane domain anchors the receptor to a cell membrane, and the intracellular domain possesses one or more tyrosine kinase enzymatic domains and interacts with downstream signal transduction molecules. The vascular endothelial growth factor receptors (VEGFRs) bind their ligand through their ECDs, which are comprised of multiple immunoglobulin-like domains (Ig-like domains). Ig-like domains are identified herein using the designation “D #.” For example “D1” refers to the first Ig-like domain of a particular receptor ECD. “D1-3” refers to a construct containing at least the first three Ig-like domains, and intervening sequence between domains 1 and 2 and 2 and 3, of a particular ligand binding molecule.

The complete ECD of VEGFRs is not required for ligand (growth factor) binding. The ECD of VEGFR-3 has six intact Ig-like domains and one cleaved Ig-like domain—D5 of VEGFR-3 is cleaved post-translationally into disulfide linked subunits leaving VEGFR-3. Veikkola, T., et al., Cancer Res. 60:203-212 (2000). In some embodiments, receptor fragments comprising at least the first three Ig-like domains for this family are sufficient to bind ligand. Soluble receptors capable of binding VEGF-C and VEGF-D, thereby inhibiting VEGF-C or VEGF-D activity or signaling via VEGFR-3, are also disclosed in WO2000/023565, WO2000/021560, WO2002/060950 and WO2005/087808, the disclosures of which are incorporated herein by reference in their entireties. Those soluble receptors, modified with the ΔN2 sequon change and optionally other modifications described herein, are contemplated as aspects of the invention.

Table 1 defines approximate boundaries of the Ig-like domains for human VEGFR-3. These boundaries are significant as the boundaries chosen can be used to form ligand binding molecules, and so can influence the binding properties of the resulting constructs.

TABLE 1
Immunoglobulin-like domains for human VEGFR-3

| | VEGFR-3 | VEGFR-3 |
|---|---|---|
| | SEQ ID NO: 1 positions | SEQ ID NO: 2 positions |
| D1 | 158-364 | 47-115 |
| D2 | 479-649 | 154-210 |
| D3 | 761-961 | 248-314 |
| D4 | 1070-1228 | 351-403 |
| D5 | 1340-1633 | 441-538 |
| D6 | 1739-1990 | 574-657 |
| D7 | 2102-2275 | 695-752 |

The complete ECD extends to about position 775 of SEQ ID NO: 2.

Soluble receptor constructs for use as a ligand binding molecule for human VEGF-C or VEGF-D preferably comprise at least one Ig-like domain of VEGFR-3 as described in Table 1, to as many as seven. The ligand binding molecule optionally will include sequence before the most N-terminally positioned Ig-like domain, optionally will include sequence beyond the most C-terminally Ig-like domain, and optionally will include sequence between the Ig-like domains as well. Variants, e.g., with one or more amino acid substitutions, additions, or deletions of an amino acid residue, are also contemplated. In some embodiments, the ligand binding molecule comprises a fragment of human VEGFR-3 comprising at least the first three Ig-like domains of human VEGFR-3.

In some embodiments, the ligand binding molecule is a polypeptide that comprises a portion of a human VEGFR-3 ECD, wherein the portion binds to one or both of human VEGF-C and human VEGF-D, and comprises at least the first, second and third Ig-like domains of the VEGFR-3 ECD, wherein the amino acid sequence of the ECD fragment of VEGFR-3 is modified from wildtype VEGFR-3 to eliminate the second putative N-linked glycosylation sequon of wildtype VEGFR-3, and wherein the polypeptide lacks VEGFR-3 Ig-like domains 4-7 and preferably any transmembrane domain and preferably any intracellular domain.

In some embodiments, the ligand binding molecule comprises a polypeptide similar or identical in amino acid sequence to a human VEGFR-3 polypeptide (SEQ ID NO: 2) or fragment thereof, with the proviso that positions of the ligand binding molecule corresponding to positions 104-106 of the human VEGFR-3 polypeptide set forth in SEQ ID NO: 2 are not identical to N-X-S or N-X-T, wherein the ligand binding molecule binds one or more growth factors selected from the group consisting of human VEGF-C and human VEGF-D. The fragment minimally comprises enough of the VEGFR-3 sequence to bind the ligand, and may comprise the complete receptor. ECD fragments are preferred. Preferred polypeptides have an amino acid sequence at least 80% identical to a ligand binding fragment thereof. Fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated. A genus of similar polypeptides can alternatively be defined by the ability of encoding polynucleotides to hybridize to the complement of a nucleotide sequence that corresponds to the cDNA sequence encoding the VEGFR-3 receptor.

The term “identity”, as known in the art, refers to a relationship between the sequences of two or more polypeptide molecules or two or more nucleic acid molecules, as determined by comparing the sequences. In the art, “identity” also means the degree of sequence relatedness nucleic acid molecules or polypeptides sequences, as the case may be, as determined by the match between strings of two or more nucleotide or two or more amino acid sequences. “Identity” measures the percent of identical matches between the smaller of two or more sequences with gap alignments (if any) addressed by particular a mathematical model of computer program (i.e., “algorithms”). Appropriate algorithms for determining the percent identities of the invention include BLASTP and BLASTN, using the most common and accepted default parameters.

Ligand binding molecules may also be described as having an amino acid sequence encoded by a nucleic acid sequence at least 80% identical to a fragment of SEQ ID NO: 1 encoding a ligand binding fragment of VEGFR-3, with the proviso that positions of the ligand binding molecule corresponding to positions 104-106 of the encoded ligand binding fragment of VEGFR-3 are not identical to N-X-S or N-X-T. Nucleic acid fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated. For example, a preferred ligand binding molecule comprises an amino acid sequence that binds human VEGF-C and/or human VEGF-D and that is encoded by a nucleotide sequence that hybridizes to the complement of SEQ ID NO: 1 under moderately or highly stringent conditions discussed herein.

In some embodiments, the ligand binding molecule comprises a polypeptide comprising a fragment of human VEGFR-3 (SEQ ID NO: 2) selected from the group consisting of positions 1-226 or 25-226 of SEQ ID NO: 2, positions 1-229 or 25-229 of SEQ ID NO: 2 and positions 1-329 or 25-229 of SEQ ID NO: 2, with the proviso that positions 104-106 of the encoded ligand binding fragment of VEGFR-3 are not identical to N-X-S or N-X-T. In some embodiments, the ligand binding molecule is a polypeptide comprising a fragment of human VEGFR-3 (SEQ ID NO: 2) selected from the group consisting of positions 47-224 of SEQ ID NO: 2, positions 47-225 of SEQ ID NO: 2, positions 47-226 of SEQ ID NO: 2, positions 47-227 of SEQ ID NO: 2, positions 47-228 of SEQ ID NO: 2, positions 47-229 of SEQ ID NO: 2, positions 47-230 of SEQ ID NO: 2, positions 47-231 of SEQ ID NO: 2, positions 47-232 of SEQ ID NO: 2, positions 47-236 of SEQ ID NO: 2, positions 47-240 of SEQ ID NO: 2, and positions 47-245 of SEQ ID NO: 2, with the proviso that positions 104-106 of the encoded ligand binding fragment of VEGFR-3 are not identical to N-X-S or N-X-T. In some embodiments, the ligand binding molecule is a polypeptide comprising a fragment of human VEGFR-3 (SEQ ID NO: 2), selected from the group consisting of positions 47-314 of SEQ ID NO: 2, positions 47-210 of SEQ ID NO: 2, and positions 47-247 of SEQ ID NO: 2, with the proviso that positions 104-106 of the encoded ligand binding fragment of VEGFR-3 are not identical to N-X-S or N-X-T.

Ligand binding molecules can also be described as having an amino acid sequence that is similar or identical to the amino acid sequence set forth in SEQ ID NO: 3. Preferred polypeptides have an amino acid sequence at least 80% identical to the amino acid sequence set forth in SEQ ID NO: 3, with the proviso that positions 80-82 of the polypeptide set forth in SEQ ID NO: 3 are not identical to N-X-S or N-X-T, wherein the ligand binding molecule binds one or more growth factors selected from the group consisting of human VEGF-C and human VEGF-D. Polypeptides that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated. A genus of similar polypeptides can alternatively be defined by the ability of encoding polynucleotides to hybridize to the complement of a nucleotide sequence that corresponds to the cDNA sequence encoding the VEGFR-3 receptor.

In some embodiments, the ligand binding molecule comprises an amino acid sequence comprising amino acids 22-290 of SEQ ID NO: 3. In some embodiments, the ligand binding molecule comprises an amino acid sequence comprising amino acids 23-290 of SEQ ID NO: 3. In some embodiments, the ligand binding molecule comprises amino acids 22-537 of SEQ ID NO: 3, or amino acids 23-537 of SEQ ID NO: 3 or amino acids 1-537 of SEQ ID NO: 3.

The term “component domain” as used herein to refers to a domain within a ligand binding molecule which is derived from or based on a protein domain within the extracellular portion of a receptor protein. For example, each Ig-domain of VEGFR-3 (D1-D7) and other tyrosine kinase receptor family members (e.g. such as VEGFR-1 and VEGFR-2) constitute component domains. Reference herein to a component domain includes both the complete native wildtype domain and also insertional, deletional and/or substitutional variants thereof which substantially retain the functional characteristics of the intact domain. It will be readily apparent to one of skill in the art that numerous variants of the above domains (e.g. Ig-domains) can be obtained which will retain substantially the same functional characteristics as the wild-type domain.

The growth factor receptors, from which ligand binding molecules may be derived, include splice variants and naturally-occurring allelic variations. Allelic variants are well known in the art, and represent alternative forms or a nucleic acid sequence that comprise substitution, deletion or addition of one or more nucleotides, but which do not result in any substantial functional alteration of the encoded polypeptide. Exemplary allelic variants of VEGFR-3 have been reported in the literature, e.g., at http<colon>//www.uniprot.org/uniprot/P35916, and include positions 149, 378, 494, 527, and 641 within the ECD. Standard methods can readily be used to generate such polypeptides including site-directed mutagenesis of polynucleotides, or specific enzymatic cleavage and ligation. Similarly, use of peptidomimetic compounds or compounds in which one or more amino acid residues are replaced by a non-naturally-occurring amino acid or an amino acid analog that retain binding activity is contemplated. Preferably, where amino acid substitution is used, the substitution is conservative, i.e. an amino acid is replaced by one of similar size and with similar charge properties. As used herein, the term “conservative substitution” denotes the replacement of an amino acid residue by another, biologically similar residue. Examples of conservative substitutions include the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic acid for aspartic acid, or glutamine for asparagine, and the like. Neutral hydrophilic amino acids that can be substituted for one another include asparagine, glutamine, serine and threonine. The term “conservative substitution” also includes the use of a substituted amino acid in place of an unsubstituted amino acid.

Alternatively, conservative amino acids can be grouped as described in Lehninger, (Biochemistry, Second Edition; Worth Publishers, Inc. NY:NY, pp. 71-77 (1975)) as set out in the following:

### Non-Polar (Hydrophobic)

A. Aliphatic: A, L, I, V, P,
B. Aromatic: F, W,
C. Sulfur-containing: M,
D. Borderline: G.

### Uncharged-Polar

A. Hydroxyl: S, T, Y,
B. Amides: N, Q,
C. Sulfhydryl: C,
D. Borderline: G.

### Positively Charged (Basic): K, R, H.

### Negatively Charged (Acidic): D, E.

For the avoidance of doubt, “component domain” includes a domain corresponding to D1 of VEGFR-3 in which the N-X-S/T sequon motif at position 104-106 of SEQ ID No: 2 has been mutated, e.g. due to substitution.

In embodiments where the ligand binding molecule comprises multiple component domains, for example component domains D1, D2 and D3 of VEGFR-3, the component domains may be connected directly to each other or may be connected via one or more spacers. Generally, the term “spacer” means one or more molecules, for example nucleic acids or amino acids, or non-peptide moieties, such as polyethylene glycol or disulfide bridges, which may be inserted between one or more component domains forming a covalent bond. Spacer sequences may be used to provide a desirable site of interest between components for ease of manipulation. A spacer may also be provided to enhance expression of the ligand binding polypeptide from a host cell, to decrease steric hindrance such that the component or group of components may assume its/their optimal tertiary structure and/or interact appropriately with its/their target molecule. For spacers and methods of identifying desirable spacers, see, for example, George et al. (2003) Protein Engineering 15:871-879, herein specifically incorporated by reference. A spacer sequence may include one or more amino acids naturally connected to a receptor component, or may be an added sequence used to enhance expression of the ligand binding polypeptides, provide specifically desired sites of interest, allow component domains to form optimal tertiary structures and/or to enhance the interaction of a component or group of components with its/their target molecule. In one embodiment, the spacer comprises one or more peptide sequences between one or more components which is (are) between 1-100 amino acids, preferably 1-50 amino acids in length. In a preferred embodiment, the spacer between two component domains substantially consists of amino acids naturally connected to the receptor component in the wildtype receptor. In the case of a ligand binding molecule comprising multiple component domains from the same receptor which domains are adjacent each other in the native receptor, such as for example D1, D2 and D3 of VEGFR-3, in one embodiment, the domains are connected to each other (e.g. D1 to D2 and D2 to D3) using spacers corresponding to the naturally-occurring amino acid linking sequences.

In some variations, each ligand binding polypeptide is expressed as a fusion with a fusion partner protein, such as an immunoglobulin constant region, and the heterologous fusion partners are linked to form the ligand binding molecule.

### Multimers, Multimerizing Components, Fusion Partners and Linkers

The fusion partner is any heterologous component that enhances the functionality of the ligand binding molecule. Thus, for example, a fusion partner may increase the solubility, modulate the clearance, facilitate targeting of particular cell or tissue types, enhance the biological activity, aid the production and/or recovery, enhance a pharmacological property or enhance a pharmacokinetic (PK) profile of the ligand binding polypeptide. With regards to enhancing the PK profile, this may be achieved by, for example, enhancing the serum half-life, tissue penetrability, lack of immunogenicity or stability of the ligand binding molecule. In preferred embodiments, a fusion partner is selected from the group consisting of a multimerizing component, a serum protein or a molecule capable of binding a serum protein. When the fusion partner is a serum protein or fragment thereof, it is selected from the group consisting of α-1-microglobulin, AGP-1, orosomuciod, α-1-acid glycoprotein, vitamin D binding protein (DBP), hemopexin, human serum albumin (hSA), transferrin, ferritin, afamin, haptoglobin, α-fetoprotein thyroglobulin, α-2-HS-glycoprotein, 3-2-glycoprotein, hyaluronan-binding protein, syntaxin, C1R, C1q a chain, galectin3-Mac2 binding protein, fibrinogen, polymeric Ig receptor (PIGR), α-2-macroglobulin, urea transport protein, haptoglobin, IGFBPs, macrophage scavenger receptors, fibronectin, giantin, Fc, α-1-antichyromotrypsin, α-1-antitrypsin, antithrombin III, apolipoprotein A-1, apolipoprotein B, β-2-microglobulin, ceruloplasmin, complement component C3 or C4, CI esterase inhibitor, C-reactive protein, cystatin C, and protein C. In a more specified embodiment, the fusion partner is selected from the group consisting of α-1-microglobulin, AGP-1, orosomuciod, α-1-acid glycoprotein, vitamin D binding protein (DBP), hemopexin, human serum albumin (hSA), afamin, and haptoglobin. The inclusion of a fusion partner component may extend the serum half-life of the fusion polypeptide of the invention when desired. See, for example, U.S. Pat. Nos. 6,423,512, 5,876,969, 6,593,295, and 6,548,653, herein specifically incorporated by reference in their entirety, for examples of serum albumin fusion polypeptides. hSA is widely distributed throughout the body, particularly in the intestinal and blood components, and has an important role in the maintenance of osmolarity and plasma volume. It is slowly cleared in the liver, and typically bas an in vivo half-life of 14-20 days in humans (Waldmann et al. (1977) Albumin, Structure Function and Uses; Pergamon Press; pp. 255-275).

When a fusion partner is a molecule capable of binding a serum protein, the molecule may be a synthetic small molecule, a lipid or liposome, a nucleic acid, including a synthetic nucleic acid such as an aptamer, a peptide, or an oligosaccharide. The molecule may further be a protein, such as, for example, FcγR1, FcγR2, FcγR3, polymeric Ig receptor (PIGR), ScFv, and other antibody fragments specific for a serum protein.

When the fusion partner is a multimerizing component, it is any natural or synthetic sequence or compound capable of operably linking a first ligand binding molecule with another ligand binding molecule or another multimerizing component of another ligand binding molecule to form a higher order structure, e.g., a dimer, a trimer, etc. Suitable multimerizing components may include a leucine zipper, including leucine zipper domains derived from c-jun or c-fos; sequences derived from the constant regions of kappa or lambda light chains; synthetic sequences such as helix-loop-helix motifs (Muller et al. (1998) FEBS Lett. 432:45-49), coil-coil motifs, etc., or other generally accepted multimerizing domains known to the art. In some embodiments, the fusion component comprises an immunoglobulin-derived domain from, for example, human IgG, IgM or IgA.

In one aspect, a ligand binding molecule described herein is produced as a multimer. Each subunit of the multimer comprises or consists of a ligand binding molecule, for example a ligand binding polypeptide. These multimers may be homodimeric, heterodimeric, or multimeric soluble receptors, with multimeric receptors consisting of 9 or fewer subunits, preferably 6 or fewer subunits, even more preferably 3 or fewer subunits, and most preferably 2 subunits. Preferably, these multimeric soluble receptors are homodimers of ligand binding molecules.

The at least two subunits in a multimer are operably linked to one another. The term “operably linked” indicates that the subunits are associated through covalent and/or non-covalent bonding. The subunits may be covalently linked by any suitable means, such as via a cross-linking reagent or a linker such as a polypeptide or peptide linker. In another embodiment, the subunits are linked via non-covalent linkages. In some variations, the two subunits (for example two ligand binding polypeptides) are attached by a peptide linkage, either directly or via a “peptide linker”. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met), for example, a 13-amino acid linker sequence consisting of Glu-Phe-Gly-Ala-Gly-Leu-Val-Leu-Gly-Gly-Gln-Phe-Met (SEQ ID NO: 7), a 15-amino acid linker sequence consisting Of (G4S)3 (SEQ ID NO: 8), a 16-amino acid linker sequence consisting of GGSGG SGGGG SGGGG S (SEQ ID NO: 9) or the hinge region of human lgG (e.g. IgGI, IgG2, IgG3 or IgG4). In some variations, the two subunits are ligand binding polypeptides comprising two distinct polypeptide chains that are linked to each other, e.g, by disulfide bonding or other bonds.

In some embodiments, the ligand binding molecule is in the form of a fusion protein comprising at least two subunits each comprising a ligand binding polypeptide. In this manner, the fusion protein can be produced recombinantly, by direct expression in a host cell of a nucleic acid molecule encoding the same as a single open reading frame.

In some variations, a ligand binding polypeptide is expressed as a fusion with a heterologous protein fusion partner, such as an immunoglobulin constant region, and the heterologous fusion partners are linked to form a multimeric ligand binding molecule. In one embodiment, the subunits are operably linked to a multimerizing component. A multimerizing component includes any natural or synthetic sequence capable of operably linking two or more subunits to form a higher order structure, e.g., a dimer, a trimer, etc. A multimerizing component may operably link two or more subunits by interacting “directly” with the subunits. Alternatively, a multimerizing component for one subunit may interact with another multimerizing component for another subunit to operably link the subunits.

In one embodiment, the subunits are operably linked to an additional amino acid domain that provides for the multimerization of the subunits (in particular the additional domains comprise any functional region providing for dimerization of the subunits). The term “operably linked” indicates that VEGFR-3-based subunit, and the additional amino acid domain are associated through peptide linkage, either directly or via a “peptide linker” (as defined herein), and the VEGFR-3-based subunit retains ligand binding properties. The additional amino acid domain may be located upstream (N-ter) or downstream (C-ter) from the VEGFR-3 subunit sequence. Preferably it is located downsteam (i.e. away from the first immunoglobulin-like domain (Ig-I domain). In this manner, the fusion protein can be produced recombinantly, by direct expression in a host cell of a nucleic acid molecule encoding the same. In such embodiments, a ligand binding molecule described herein is a multimer of fusion proteins containing ligand binding polypeptides and a multimerizing component capable of interacting with the multimerizing component present in another fusion protein to form a higher order structure, such as a dimer. These type of fusion proteins may be prepared by operably linking the VEGFR-3 subunit sequence (i.e., ligand binding polypeptide) to domains isolated from other proteins allowing the formation of dimers, trimers, etc. Examples for protein sequences allowing the multimerization of the ligand polypeptides described herein include, but are not limited to, domains isolated from proteins such as immunoglobulins, hCG (WO 97/30161), collagen X (WO 04/33486), C4BP (WO 04/20639), Erb proteins (WO 98/02540) or coiled coil peptides (WO 01/00814), the disclosure of which are incorporated herein by reference in their entireties.

The multimerizing component may, for example, be selected from (i) an amino acid sequences between 1 to about 500 amino acids in length, (ii) leucine zippers, (iii) helix loop motifs and (iv) coil-coil motifs. When the multimerizing component comprises an amino acid sequence between 1 to about 500 amino acids in length, the sequence may contain one or more cysteine residues capable of forming a disulfide bond with a corresponding cysteine residue on another fusion polypeptide comprising a multimerizing component with one or more cysteine residues.

In a particular aspect, the multimers are dimers of ligand binding polypeptides where the polypeptides are operably linked to an immunoglobulin or a portion of an immunoglobulin as the fusion partner, which may also act as the multimerizing component. The term “operably linked” indicates that the ligand binding polypeptides and the immunoglobulin or portion thereof are associated through peptide linkage, either directly or via a “peptide linker” (as defined herein), and ligand binding properties of the ligand binding polypeptides are retained. In this embodiment, the ligand binding polypeptides are operably linked to all or a portion of an immunoglobulin, particularly a human immunoglobulin, even more particularly the Fc portion of a human immunoglobulin. Typically an Fc portion of a human immunoglobulin contains two constant region domains (the CH2 and CH3 domains) and a hinge region but lacks the variable region. (See e.g. U.S. Pat. Nos. 6,018,026 and 5,750,375, incorporated herein by reference.) The immunoglobulin may be selected from any of the major classes of immunoglobulins, including IgA, IgD, IgE, IgG and IgM, and any subclass or isotype, e.g. IgG1, IgG2, IgG3 and IgG4; IgA-I and IgA-2. In an embodiment, the Fc moiety is of human IgG4, which is stable in solution and has little or no complement activating activity. In another embodiment, the Fc moiety is of human IgGI. The Fc part may be mutated in order to prevent unwanted activities, such as complement binding, binding to Fc receptors, or the like. The amino acid sequence derived from the immunoglobulin may be linked to the C-terminus or to the N-terminus of the ligand binding polypeptide, preferably to the C-terminus. Such fusion proteins can be prepared by transfecting cells with DNA encoding VEGFR-3 subunit:Fc fusion protein and expressing the dimers in the same cells. In a particular embodiment, the ligand binding polypeptides are the same on each monomer subunit (i.e the dimer is a homodimer).

Methods for making immunoglobulin fusion proteins are well known in the art, such as the ones described in Hollenbaugh and Aruffo (“Construction of Immunoglobulin Fusion Proteins”, in Current Protocols in Immunology, Suppl. 4, pages 10.19.1-10.19.11, 1992) or WO 01/03737, for example, both incorporated herein by reference.

Alternatively, the dimers of ligand binding polypeptides of the present invention can be prepared by operably linking one of the ligand binding polypeptides to the constant region of an immunoglobulin heavy chain and operably linking the other ligand binding polypeptide to the constant region of an immunoglobulin light chain. For example, a ligand binding polypeptide can be operably linked to the CH1-hinge-CH2-CH3 region of human IgG1 and another or the same ligand binding polypeptide can be operably linked to the C kappa region of the Ig kappa light chain. In an embodiment, the heavy constant chain is human y4, which is stable in solution and has little or no complement activating activity. In another embodiment, the heavy constant chain is human γI. The heavy constant chain may be mutated in order to prevent unwanted activities, such as complement binding, binding to Fc receptors, or the like. Also, if needed, fusion proteins described herein may comprise any functional region facilitating purification or production. Specific examples of such additional amino acid sequences include a GST sequence or a His tag sequence. In some variations, the region facilitating purification is removed for formulation of a composition for pharmaceutical use.

The amino acid sequence derived from the immunoglobulin may be linked to the C-terminus or to the N-terminus of the ligand binding polypeptide, preferably to the C-terminus. Cells transfected with DNA encoding the immunoglobulin light chain fusion protein and the immunoglobulin heavy chain fusion protein express heavy chain/light chain heterodimers containing each a ligand binding polypeptide. Both ligand binding polypeptides advantageously comprise a native or heterologous signal peptide when initially synthesized, to promote secretion from the cell, but the signal sequence is cleaved upon secretion. Variations of any of the foregoing embodiments that include the signal peptide are contemplated. The native signal peptide of human VEGFR-3 comprises residues 1-24 of SEQ ID NO: 2. Numerous other signal peptide proteins are taught in the literature.

In another particular aspect of the present invention, ligand binding polypeptides of the multimers are linked via non-covalent linkages. Non-covalent bonding of the subunits may be achieved by any suitable means that does not interfere with its biological activity (i.e. its ability to bind human VEGF-C and/or VEGF-D). In a particular aspect, these multimers are dimers of ligand binding polypeptides where one ligand binding polypeptide is operably linked to a first compound and another or the same ligand binding polypeptide is operably linked to a second compound that will non-covalently bond to the first compound. Examples of such compounds are biotin and avidin. The dimers of ligand binding polypeptides can be prepared by operably linking one VEGFR-3 subunit to biotin and operably linking the other ligand binding polypeptide to avidin. The receptor is thus formed through the non-covalent interactions of biotin with avidin. Other examples include subunits of heterodimeric proteinaceous hormone. In these embodiments, a DNA construct encoding one ligand binding protein is fused to a DNA construct encoding a subunit of a heterodimeric proteinaceous hormone, such as hCG, and a DNA construct encoding the other ligand binding polypeptide is fused to DNA encoding the other subunit of the heterodimeric proteinaceous hormone, such as hCG (as disclosed in U.S. Pat. No. 6,193,972). These DNA constructs are co-expressed in the same cells leading to the expression of a ligand binding molecule, as each co-expressed sequence contains a corresponding hormone subunit so as to form a heterodimer upon expression. The amino acid sequence derived from the heterodimeric proteinaceous hormone may be linked to the C-terminus or to the N-terminus of the ligand binding polypeptides, preferably to the C-terminus. Both subunits advantageously comprise a native or heterologous signal peptide when initially synthesized, to promote secretion from the cell, but the signal sequence is cleaved upon secretion.

In one embodiment, the ligand binding molecule is operably linked to a non-VEGFR-3 derived binding unit, i.e. a binding unit which contains no component domains derived from VEGFR-3. Such chimeric ligand binding molecules may, for example, comprise heterologous binding units based on other tyrosine kinase receptors. In one embodiment, such heterologous binding units bind to at least one ligand polypeptide selected from VEGF-A (VEGF), VEGF-B, PIGF, PDGF-A, PDGF-B, PDGF-C and PDGF-D. In a preferred embodiment, such heterologous binding units bind to at least VEGF-A (VEGF).

In one embodiment, such heterologous binding units comprise component domains derived from VEGFR-1 or VEGFR-2 or both. An examples of heterologous binding units which may be employed, in combination with the ligand binding molecules of the present invention in the form of chimeric ligand binding molecules, include the VEGF-trap molecules described in, for example, WO 2000/75319, WO 2005/000895 and WO 2006/088650. A preferred heterologous binding unit comprises Ig-domain 2 from VEGFR-1 (R1D2) and Ig-domain 3 from VEGFR-2 (R2D3), optionally fused to an Fc portion an immunoglobulin. In one embodiment is envisaged a chimeric molecule comprising a ligand binding polypeptide of the present invention linked to an Fc portion of an immunoglobulin operably linked with a R1D2R2D3 binding unit fused to an Fc portion an immunoglobulin. The two binding units are operably linked via disulphide bonding between the two Fc portions.

### Linkers

While Ig-like domains of human VEGFR-3 may be directly attached to one another (via a peptide, disulfide or other type of covalent bond) or to Ig-like domains of other receptors, the ligand binding molecules described herein optionally further comprise a (one or more) linker that connects together two or more different binding units, e.g., VEGFR-3 ECD fragments with another VEGFR-3 ECD fragment, or even a copy of itself. A linker may also link a binding unit to other substituents described herein. In some embodiments, the linker comprises a heterologous polypeptide. For example, in some embodiments, the linker comprises a peptide that links the binding units to form a single continuous peptide that can be expressed as a single ligand binding molecule. Linkers may be chosen such that they are less likely to induce an allergic reaction. Polysaccharides or other moieties also may be used to link binding units to form a ligand binding molecule.

More than one linker may be used per ligand binding molecule. The linker may be selected for optimal conformational (steric) freedom between the various ligand binding units to allow them to interact with each other if desired, e.g., to form dimers, or to allow them to interact with ligand. The linker may be linear such that consecutive binding units are linked in series, or the linker may serve as a scaffold to which various binding units are attached, e.g., a branched linker. A linker may also have multiple branches, e.g., as disclosed in Tam, J. Immunol. Methods 196:17 (1996). Binding units may be attached to each other or to the linker scaffold via N-terminal amino groups, C-terminal carboxyl groups, side chains, chemically modified groups, side chains, or other means.

Linker peptides may be designed to have sequences that permit desired characteristics. For example, the use of glycyl residues allow for a relatively large degree of conformational freedom, whereas a proline would tend to have the opposite effect. Peptide linkers may be chosen so that they achieve particular secondary and tertiary structures, e.g., alpha helices, beta sheets or beta barrels. Quaternary structure can also be utilized to create linkers that join two binding units together non-covalently. For example, fusing a protein domain with a hydrophobic face to each binding unit may permit the joining of the two binding units via the interaction between the hydrophobic interaction of the two molecules. In some embodiments, the linker may provide for polar interactions. For example, a leucine zipper domain of the proto-oncoproteins Myc and Max, respectively, may be used. Luscher and Larsson, Ongogene 18:2955-2966 (1999). In some embodiments, the linker allows for the formation of a salt bridge or disulfide bond. Linkers may comprise non-naturally occurring amino acids, as well as naturally occurring amino acids that are not naturally incorporated into a polypeptide.

In some embodiments, the linker comprises a coordination complex between a metal or other ion and various residues from the multiple peptides joined thereby.

Linear peptide linkers of at least one amino acid residue are contemplated. In some embodiments the linker has more than 10,000 residues. In some embodiments the linker has from 1-10,000 residues, 1-1000 residues, 1-100 residues, 1-50 residues, or 1-10 residues. In some embodiments, the linear peptide linker comprises residues with relatively inert side chains. Peptide linker amino acid residues need not be linked entirely or at all via alpha-carboxy and alpha-amino groups. That is, peptides may be linked via side chain groups of various residues.

The linker may affect whether the polypeptide(s) to which it is fused to is able to dimerize to each other or to another polypeptide. The linker serves a number of functions. Native receptor monomers restrained to the roughly two-dimensional plane of the cell membrane enjoy a relatively high local concentration and in the availability of co-receptors (binding units), increasing the probability of finding a partner. Receptors free in solution lacking such advantages may be aided by a linker that increases the effective concentration of the monomers.

In some embodiments, a ligand binding molecule may comprise more than one type of linker. Suitable linkers may also comprise the chemical modifications discussed above.

The ligand binding molecules described herein may comprise an additional N-terminal amino acid residue, preferably a methionine. Indeed, depending on the expression system and conditions, polypeptides may be expressed in a recombinant host cell with a starting Methionine. This additional amino acid may then be either maintained in the resulting recombinant protein, or eliminated by means of an exopeptidase, such as Methionine Aminopeptidase, according to methods disclosed in the literature (Van Valkenburgh H A and Kahn R A, Methods Enzymol. (2002) 344:186-93; Ben-Bassat A, Bioprocess Technol. (1991) 12:147-59).

### Substituents and Other Chemical Modifications

The ligand binding molecules described herein are optionally chemically modified with various substituents. Such modifications preferably do not substantially reduce the growth factor binding affinities or specificities of the ligand binding molecule. Rather, the chemical modifications impart additional desirable characteristics as discussed herein. Chemical modifications may take a number of different forms such as heterologous peptides, polysaccharides, lipids, radioisotopes, non-standard amino acid resides and nucleic acids, metal chelates, and various toxins.

The receptor fragments (or “binding units” or “component domains”) and ligand binding molecules described herein are optionally fused to heterologous fusion partners such as heterologous polypeptides to confer various properties, e.g., increased solubility, modulation of clearance, targeting to particular cell or tissue types. In some embodiments, the receptor fragment is linked to an Fc domain of IgG or other immunoglobulin. In some embodiments, a receptor fragment is fused to alkaline phosphatase (AP). Methods for making Fc or AP fusion constructs are found in WO 02/060950. By fusing the ligand binding polypeptide or molecule with protein domains that have specific properties (e.g. half-life, bioavailability, interaction partners) it is possible to confer these properties to the ligand binding molecule (e.g., the molecules is engineered to have a specific tissue distribution or specific biological half-life).

In some embodiments, ligand binding molecule includes a co-receptor and a VEGFR fragment.

The particular fusion partner (e.g., heterologous polypeptide) used in a particular ligand binding molecule can influence whether or not a VEGR-3 fragment will dimerize, which in turn may affect ligand binding.

For substituents such as an Fc region of human IgG, the fusion can be fused directly to a ligand binding molecule or fused through an intervening sequence. For example, a human IgG hinge, CH2 and CH3 region may be fused at either the N-terminus or C-terminus of a ligand binding molecule to attach the Fc region. The resulting Fc-fusion construct enables purification via a Protein A affinity column (Pierce, Rockford, III.). Peptide and proteins fused to an Fc region can exhibit a substantially greater half-life in vivo than the unfused counterpart. A fusion to an Fc region allows for dimerization/multimerization of the fusion polypeptide. The Fc region may be a naturally occurring Fc region, or may be modified for superior characteristics, e.g., therapeutic qualities, circulation time, reduced aggregation.

Polypeptides can be modified, for instance, by glycosylation, amidation, carboxylation, or phosphorylation, or by the creation of acid addition salts, amides, esters, in particular C-terminal esters, and N-acyl derivatives. Ig-like domains I-III of VEGFR-3 comprises 5 putative N-glycosylation sites (referred to herein as N1, N2, N3, N4 and N5 sequons or regions of VEGFR-3, respectively). N1 corresponds to amino acids 33-35 of SEQ ID NO: 2; N2 corresponds to amino acids 104-106 of SEQ ID NO: 2; N3 corresponds to amino acids 166-168 of SEQ ID NO: 2; N4 corresponds to amino acids 251-253 of SEQ ID NO: 2 and N5 corresponds to amino acids 299-301 of SEQ ID NO: 2. In some embodiments, a ligand binding molecule described herein comprises a modification in the N2 region of the molecule. For example, in some embodiments, the amino acid in the ligand binding molecule corresponding to position 104 of SEQ ID NO: 2 is deleted and replaced with another amino acid. Conservative substitutions are preferred. In some embodiments, the amino acid corresponding to position 104 of SEQ ID NO: 2 is deleted and replaced with an amino acid selected from the group consisting of glutamine, aspartate, glutamate, arginine and lysine. In still other variations, position 106 is substituted to eliminate the N2 sequon. In embodiments where the N2 sequon of SEQ ID NO: 2 is modified as described above, the N1, N3, N4 and N5 sequons of SEQ ID NO: 2 are preferably unmodified.

The proteins also can be modified to create peptide derivatives by forming covalent or noncovalent complexes with other moieties. Covalently bound complexes can be prepared by linking the chemical moieties to functional groups on the side chains of amino acids of the polypeptides, or at the N-or C-terminus.

Polypeptides can be conjugated to a reporter group, including, but not limited to a radiolabel, a fluorescent label, an enzyme (e.g., that catalyzes a calorimetric or fluorometric reaction), a substrate, a solid matrix, or a carrier (e.g., biotin or avidin). Examples of analogs are described in WO 98/28621 and in Olofsson, et al., Proc. Nat'l. Acad. Sci. USA, 95:11709-11714 (1998), U.S. Pat. Nos. 5,512,545, and 5,474,982; U.S. patents application Ser. Nos. 20020164687 and 20020164710.

Cysteinyl residues most commonly are reacted with haloacetates (and corresponding amines), such as chloroacetic acid or chloroacetamide, to give carboxymethyl or carbocyamidomethyl derivatives. Cysteinyl residues also are derivatized by reaction with bromotrifluoroacetone, α-bromo-β(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, orchloro-7-nitrobenzo-2-oxa-1,3-diazole.

Histidyl residues are derivatized by reaction with diethylprocarbonate at pH 5.5-7.0 because this agent is relatively specific for the histidyl side chain. Para-bromophenacyl bromide also is useful; the reaction is preferably performed in 0.1M sodium cacodylate at pH 6.0.

Lysinyl and amino terminal residues are reacted with succinic or carboxylic acid anhydrides. Derivatization with these agents has the effect of reversing the charge of the lysinyl residues. Other suitable reagents for derivatizing α-amino-containing residues include imidoesters such as methyl picolinimidate; pyridoxal phosphate; pyridoxal; chloroborohydride; trinitrobenzenesulfonic acid; O-methylissurea; 2,4 pentanedione; and transaminase catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pK of the guanidine functional group. Furthermore, these reagents may react with the groups of lysine as well as the arginine epsilon-amino group.

The specific modification of tyrosyl residues per se has been studied extensively, with particular interest in introducing spectral labels into tyrosyl residues by reaction with aromatic diazonium compounds or tetranitromethane. Most commonly, N-acetylimidizol and tetranitromethane are used to form O-acetyl tyrosyl species and 3-nitro derivatives, respectively. Tyrosyl residues are iodinated using 1251 or 1311 to prepare labeled proteins for use in radioimmunoassay.

Carboxyl side groups (aspartyl or glutamyl) are selectively modified by reaction with carbodiimides (R1) such as 1-cyclohexyl-3-(2-morpholinyl-(4-ethyl) carbodiimide or 1-ethyl-3 (4 azonia 4,4-dimethylpentyl)carbodiimide. Furthermore, aspartyl and glutamyl residues are converted to asparaginyl and glutaminyl residues by reaction with ammonium ions.

Derivatization with bifunctional agents is useful for crosslinking the ligand binding molecule to water-insoluble support matrixes. Such derivation may also provide the linker that may connect adjacent binding elements in a ligand binding molecule, or a binding elements to a heterologous peptide, e.g., a Fc fragment. Commonly used crosslinking agents include, e.g., 1,1-bis(diazoacetyl)-2-phenylethane, glutaraldehyde, N-hydroxysuccinimide esters, for example, esters with 4-azidosalicylic acid, homo-bifunctional imidoesters, including disuccinimidyl esters such as 3,3′-dithiiobis(succinimidylpropioonate), and bifunctional maleimides such as bis-N-maleimido-1,8-octane. Derivatizing agents such as methyl-3-[(p-azidophenyl) dithio] propioimidate yield photoactivatable intermediates that are capable of forming cross links in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440, incorporated herein by reference, are employed for protein immobilization.

Glutaminyl and asparaginyl residues are frequently deamidated to the corresponding glutamyl and aspartyl residues. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the a-amino groups of lysine, arginine, and histidine side chains (T. E. Creighton, Proteins: Structure and Molecule Properties, W. H. Freeman & Co., San Francisco, pp. 79-86, 1983), acetylation of the N-terminal amine, and, in some instances, amidation of the C-terminal carboxyl groups. Such derivatives are chemically modified polypeptide compositions in which the ligand binding molecule polypeptide is linked to a polymer. The polymer selected is typically water soluble so that the protein to which it is attached does not precipitate in an aqueous environment, such as a physiological environment. The polymer selected is usually modified to have a single reactive group, such as an active ester for acylation or an aldehyde for alkylation, so that the degree of polymerization may be controlled as provided for in the present methods. The polymer may be of any molecular weight, and may be branched or unbranched. Included within the scope of the ligand binding molecule polypeptide polymers is a mixture of polymers. Preferably, for therapeutic use of the end-product preparation, the polymer will be pharmaceutically acceptable.

The polymers each may be of any molecular weight and may be branched or unbranched. The polymers each typically have an average molecular weight of between about 2 kDa to about 100 kDa (the term “about” indicating that in preparations of a water soluble polymer, some molecules will weigh more, some less, than the stated molecular weight). The average molecular weight of each polymer is between about 5 kDa and about 50 kDa, more preferably between about 12 kDa to about 40 kDa and most preferably between about 20 kDa to about 35 kDa.

Suitable water soluble polymers or mixtures thereof include, but are not limited to, N-linked or O-linked carbohydrates, sugars, phosphates, carbohydrates; sugars; phosphates; polyethylene glycol (PEG) (including the forms of PEG that have been used to derivatize proteins, including mono-(C1-C10) alkoxy- or aryloxy-polyethylene glycol); monomethoxy-polyethylene glycol; dextran (such as low molecular weight dextran, of, for example about 6 kD), cellulose; cellulose; other carbohydrate-based polymers, poly-(N-vinyl pyrrolidone)polyethylene glycol, propylene glycol homopolymers, a polypropylene oxide/ethylene oxide co-polymer, polyoxyethylated polyols (e.g., glycerol) and polyvinyl alcohol. Also encompassed by the present invention are bifunctional crosslinking molecules which may be used to prepare covalently attached multimers.

In general, chemical derivatization may be performed under any suitable condition used to react a protein with an activated polymer molecule. Methods for preparing chemical derivatives of polypeptides will generally comprise the steps of (a) reacting the polypeptide with the activated polymer molecule (such as a reactive ester or aldehyde derivative of the polymer molecule) under conditions whereby the ligand binding molecule becomes attached to one or more polymer molecules, and (b) obtaining the reaction product(s). The optimal reaction conditions will be determined based on known parameters and the desired result. For example, the larger the ratio of polymer molecules:protein, the greater the amount of attached polymer molecule. In one embodiment, the ligand binding molecule polypeptide derivative may have a single polymer molecule moiety at the amino terminus. (See, e.g., U.S. Pat. No. 5,234,784).

A particularly preferred water-soluble polymer for use herein is polyethylene glycol (PEG). As used herein, polyethylene glycol is meant to encompass any of the forms of PEG that can be used to derivatize other proteins, such as mono-(C1-C10) alkoxy- or aryloxy-polyethylene glycol. PEG is a linear or branched neutral polyether, available in a broad range of molecular weights, and is soluble in water and most organic solvents. PEG is effective at excluding other polymers or peptides when present in water, primarily through its high dynamic chain mobility and hydrophilic nature, thus creating a water shell or hydration sphere when attached to other proteins or polymer surfaces. PEG is nontoxic, non-immunogenic, and approved by the Food and Drug Administration for internal consumption.

Proteins or enzymes when conjugated to PEG have demonstrated bioactivity, non-antigenic properties, and decreased clearance rates when administered in animals. F. M. Veronese et al., Preparation and Properties of Monomethoxypoly(ethylene glycol)-modified Enzymes for Therapeutic Applications, in J. M. Harris ed., Poly(Ethylene Glycol) Chemistry—Biotechnical and Biomedical Applications, 127-36, 1992, incorporated herein by reference. These phenomena are due to the exclusion properties of PEG in preventing recognition by the immune system. In addition, PEG has been widely used in surface modification procedures to decrease protein adsorption and improve blood compatibility. S. W. Kim et al., Ann. N.Y. Acad. Sci. 516: 116-30 1987; Jacobs et al., Artif. Organs 12: 500-501, 1988; Park et al., J. Poly. Sci, Part A 29:1725-31, 1991, incorporated herein by reference. Hydrophobic polymer surfaces, such as polyurethanes and polystyrene can be modified by the grafting of PEG (MW 3,400) and employed as nonthrombogenic surfaces. Surface properties (contact angle) can be more consistent with hydrophilic surfaces, due to the hydrating effect of PEG. More importantly, protein (albumin and other plasma proteins) adsorption can be greatly reduced, resulting from the high chain motility, hydration sphere, and protein exclusion properties of PEG.

PEG (MW 3,400) was determined as an optimal size in surface immobilization studies, Park et al., J. Biomed. Mat. Res. 26:739-45, 1992, while PEG (MW 5,000) was most beneficial in decreasing protein antigenicity. (F. M. Veronese et al., In J. M. Harris, et al., Poly(Ethylene Glycol) Chemistry—Biotechnical and Biomedical Applications, 127-36.)

Methods for preparing pegylated ligand binding molecules will generally comprise the steps of (a) reacting the polypeptide with polyethylene glycol (such as a reactive ester or aldehyde derivative of PEG) under conditions whereby the ligand molecule becomes attached to one or more PEG groups, and (b) obtaining the reaction product(s). In general, the optimal reaction conditions for the acylation reactions will be determined based on known parameters and the desired result. For example, the larger the ratio of PEG: protein, the greater the percentage of poly-pegylated product. In some embodiments, the ligand binding molecule will have a single PEG moiety at the N-terminus. See U.S. Pat. No. 8,234,784, herein incorporated by reference. In some embodiments, a ligand binding molecule described herein optionally comprises at least one PEG moiety attached to the molecule. For example, in some embodiments, PEG of about 20-40 kDa is attached to the amino terminus of the ligand binding molecule.

Derivatized ligand binding molecules disclosed herein may have additional activities, enhanced or reduced biological activity, or other characteristics, such as increased or decreased half-life, as compared to the non-derivatized molecules.

### Polynucleotides Encoding Ligand Binding Molecules and Expression Systems

The invention comprises not only the ligand binding molecules, binding units, and polypeptides described herein, but also nucleic acids encoding such molecules, vectors comprising such molecules and host cells comprising such vectors. Methods employing any of the molecules, units, polypeptides, nucleic acids, vectors and hosts cells are all considered aspects of the invention.

An exemplary human VEGFR-3 coding sequence is set forth in SEQ ID NO: 1, and fragments of SEQ ID NO: 1 (modified at the N2 sequon) are contemplated as coding sequences for ligand binding polypeptides described herein. (For example, fragments encoding all or portions of the VEGFR-3 ECD are contemplated.) Due to the well-known degeneracy of the genetic code, numerous equivalent coding sequences are possible for any polypepide-encoding sequence, and all such equivalents are contemplated as aspects of the invention.

Furthermore, just as amino acid sequence variation from VEGFR-3 wild type ECD is contemplated, as described above, nucleic acid sequence variation is also contemplated. The nucleic acid sequence variation can be characterized as percent identity relative to SEQ ID NO: 1 (e.g. at least 80, 85, 90, 92, 93, 94, 95, 96, 97, 98, or 99% identity).

Nucleotide sequence variation also can be characterized by ability to hybridize to the complement of a preferred coding sequence. Nucleic acid molecules include those molecules which comprise nucleotide sequences which hybridize under moderately or highly stringent conditions as defined herein with the ECD-encoding sequence of the nucleic acid molecule set forth in SEQ ID NO: 1, or of a molecule encoding a polypeptide, which polypeptide comprises the receptor tyrosine kinase amino acids sequence set forth in SEQ ID NOs: 2 and 3, or of a nucleic acid fragment as described herein, or of a nucleic acid fragment encoding a polypeptide as described herein.

The term “highly stringent conditions” refers to those conditions that are designed to permit hybridization of DNA strands whose sequences are highly complementary, and to exclude hybridization of significantly mismatched DNAs. Hybridization stringency is principally determined by temperature, ionic strength, and the concentration of denaturing agents such as formamide. Examples of “highly stringent conditions” for hybridization and washing are 0.015 M sodium chloride, 0.0015 M sodium citrate at 65-68° C. or 0.015 M sodium chloride, 0.0015 M sodium citrate, and 50% formamide at 42° C. See Sambrook, Fritsch & Maniatis, Molecular Cloning: A Laboratory Manual, 2nd Ed., Cold Spring Harbor Laboratory, (Cold Spring Harbor, N.Y. 1989); and Anderson et al., Nucleic Acid Hybridization: a Practical approach, Ch. 4, IRL Press Limited (Oxford, England). Limited, Oxford, England. Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate (NaDodSO₄ or SDS), ficoII, Denhardt's solution, sonicated salmon sperm DNA (or another non-complementary DNA), and dextran sulfate, although other suitable agents can also be used.

The concentration and types of these additives can be changed without substantially affecting the stringency of the hybridization conditions. Hybridization experiments are usually carried out at pH 6.8-7.4; however, at typical ionic strength conditions, the rate of hybridization is nearly independent of pH. See Anderson et al., Nucleic Acid Hybridization: a Practical Approach, Ch. 4, IRL Press Limited (Oxford, England).

Factors affecting the stability of a DNA duplex include base composition, length, and degree of base pair mismatch. Hybridization conditions can be adjusted by one skilled in the art in order to accommodate these variables and allow DNAs of different sequence relatedness to form hybrids. The melting temperature of a perfectly matched DNA duplex can be estimated by the following equation:

Tm(° C.)=81.5+16.6(log[Na+])+0.41(% G+C)−600/N−0.72(% formamide)

where N is the length of the duplex formed, [Na+] is the molar concentration of the sodium ion in the hybridization or washing solution, % G+C is the percentage of (guanine+cytosine) bases in the hybrid. For imperfectly matched hybrids, the melting temperature is reduced by approximately 1° C. for each 1% mismatch.

The term “moderately” stringent conditions”” refers to conditions under which a DNA duplex with a greater degree of base pair mismatching than could occur under “highly stringent conditions” is able to form. Examples of typical “moderately stringent conditions” are 0.015 M sodium chloride, 0.0015 M sodium citrate at 50-65° C. or 0.015 M sodium chloride, 0.0015 M sodium citrate and 20% formamide at 37-50° C. By way of example, a “moderately stringent” condition of 50° C. in 0.015 M sodium ion will allow about a 21% mismatch.

A good estimate of the melting temperature in 1M NaCl* for oligonucleotide probes up to about 20 nt is given by:

Tm=2° C. per A−T base pair+4° C. per G−C base pair

*The sodium ion concentration in 6× salt sodium citrate (SSC) is 1 M. See Suggs et al., Developmental Biology Using Purified Genes, p. 683, Brown and Fox (eds.) (1981).

High stringency washing conditions for oligonucleotides are usually at a temperature of 0-5° C. below the Tm of the oligonucleotide in 6× SSC, 0.1% SDS.

Differences in the nucleic acid sequence may result in conservative and/or non-conservative modifications of the amino acid sequence relative to the amino acid sequence of SEQ ID NO: 2 or SEQ ID NO: 3. The invention is also directed to an isolated and/or purified DNA that corresponds to, or that hybridizes under stringent conditions with, any one of the foregoing DNA sequences.

A nucleic acid molecule encoding all or part of a polypeptide of the invention such as a ligand binding molecule or binding unit described herein can be made in a variety of ways, including, without limitation, chemical synthesis, cDNA or genomic library screening, expression library screening, and/or PCR amplification of cDNA or genomic DNA. These methods and others useful for isolating such DNA are set forth, for example, by Sambrook, et al., “Molecular Cloning: A Laboratory Manual,” Cold Spring Harbor Laboratory Press, Cold Spring Harbor, N.Y. (1989), by Ausubel, et al., eds., “Current Protocols In Molecular Biology,” Current Protocols Press (1994), and by Berger and Kimmel, “Methods In Enzymology: Guide To Molecular Cloning Techniques,” vol. 152, Academic Press, Inc., San Diego, Calif. (1987). Preferred nucleic acid sequences are mammalian sequences, such as human, rat, and mouse.

Chemical synthesis of nucleic acid molecules can be accomplished using methods well known in the art, such as those set forth by Engels, et al., Angew. Chem. Intl. Ed., 28:716-734 (1989). These methods include, inter alia, the phosphotriester, phosphoramidite and H-phosphonate methods of nucleic acid synthesis. Nucleic acids larger than about 100 nucleotides in length can be synthesized as several fragments, each fragment being up to about 100 nucleotides in length. The fragments can then be ligated together, as described below, to form the full length nucleic acid of interest. A preferred method is polymer-supported synthesis using standard phosphoramidite chemistry.

The term “vector” refers to a nucleic acid molecule amplification, replication, and/or expression vehicle, often derived from or in the form of a plasmid or viral DNA or RNA system, where the plasmid or viral DNA or RNA is functional in a selected host cell, such as bacterial, yeast, plant, invertebrate, and/or mammalian host cells. The vector may remain independent of host cell genomic DNA or may integrate in whole or in part with the genomic DNA. The vector will contain all necessary elements so as to be functional in any host cell it is compatible with. Such elements are set forth below.

Where nucleic acid encoding a polypeptide or fragment thereof has been isolated, it is preferably inserted into an amplification and/or expression vector in order to increase the copy number of the gene and/or to express the encoded polypeptide in a suitable host cell and/or to transform cells in a target organism (to express the polypeptide in vivo). Numerous commercially available vectors are suitable, though “custom made” vectors may be used as well. The vector is selected to be functional in a particular host cell or host tissue (i.e., for replication and/or expression). The polypeptide or fragment thereof may be amplified/expressed in prokaryotic and/or eukaryotic host cells, e.g,, yeast, insect (baculovirus systems), plant, and mammalian cells. Selection of the host cell will depend at least in part on whether the polypeptide or fragment thereof is to be glycosylated. If so, yeast, insect, or mammalian host cells are preferable; yeast and mammalian cells will glycosylate the polypeptide if a glycosylation site is present on the amino acid sequence.

Typically, the vectors used in any of the host cells will contain 5′ flanking sequence and other regulatory elements such as an enhancer(s), a promoter, an origin of replication element, a transcriptional termination element, a complete intron sequence containing a donor and acceptor splice site, a signal peptide sequence, a ribosome binding site element, a polyadenylation sequence, a polylinker region for inserting the nucleic acid encoding the polypeptide to be expressed, and a selectable marker element. Optionally, the vector may contain a “tag” sequence, i.e., an oligonucleotide sequence located at the 5′ or 3′ end of the coding sequence that encodes polyHis (such as hexaHis) or another small immunogenic sequence. This tag will be expressed along with the protein, and can serve as an affinity tag for purification of the polypeptide from the host cell. Optionally, the tag can subsequently be removed from the purified polypeptide by various means such as using a selected peptidase. The vector/expression construct may optionally contain elements such as a 5′ flanking sequence, an origin of replication, a transcription termination sequence, a selectable marker sequence, a ribosome binding site, a signal sequence, and one or more intron sequences. The 5′ flanking sequence may be homologous (i.e., from the same species and/or strain as the host cell), heterologous (i.e., from a species other than the host cell species or strain), hybrid (i.e., a combination of 5′ flanking sequences from more than one source), synthetic, or it may be the native polypeptide 5′ flanking sequence. As such, the source of the 5′ flanking sequence may be any unicellular prokaryotic or eukaryotic organism, any vertebrate or invertebrate organism, or any plant, provided that the 5′ flanking sequence is functional in, and can be activated by, the host cell machinery.

A transcription termination element is typically located 3′ to the end of the polypeptide coding sequence and serves to terminate transcription of the polypeptide. Usually, the transcription termination element in prokaryotic cells is a G-C rich fragment followed by a poly T sequence. Such elements can be cloned from a library, purchased commercially as part of a vector, and readily synthesized.

Selectable marker genes encode proteins necessary for the survival and growth of a host cell in a selective culture medium. Typical selectable marker genes encode proteins that (a) confer resistance to antibiotics or other toxins, e.g., ampicillin, tetracycline, or kanamycin for prokaryotic host cells, (b) complement auxotrophic deficiencies of the cell; or (c) supply critical nutrients not available from complex media.

A ribosome binding element, commonly called the Shine-Dalgarno sequence (prokaryotes) or the Kozak sequence (eukaryotes), is necessary for translation initiation of mRNA. The element is typically located 3′ to the promoter and 5′ to the coding sequence of the polypeptide to be synthesized. The Shine-Dalgarno sequence is varied but is typically a polypurine (i.e., having a high A-G content). Many Shine-Dalgarno sequences have been identified, each of which can be readily synthesized using methods set forth above.

All of the elements set forth above, as well as others useful in this invention, are well known to the skilled artisan and are described, for example, in Sambrook, et al., “Molecular Cloning: A Laboratory Manual,” Cold Spring Harbor Laboratory Press, Cold Spring Harbor, N.Y. (1989) and Berger, et al., eds., “Guide To Molecular Cloning Techniques,” Academic Press, Inc., San Diego, Calif. (1987].

For those embodiments of the invention where the recombinant polypeptide is to be secreted, a signal sequence is preferably included to direct secretion from the cell where it is synthesized. Typically, the polynucleotide encoding the signal sequence is positioned at the 5′ end of the coding region. Many signal sequences have been identified, and any of them that are functional in a target cell or species may be used in conjunction with the transgene. In many cases, gene transcription is increased by the presence of one or more introns on the vector. The intron may be naturally-occurring, especially where the transgene is a full length or a fragment of a genomic DNA sequence. The intron may be homologous or heterologous to the transgene and/or to the transgenic mammal into which the gene will be inserted. The position of the intron with respect to the promoter and the transgene is important, as the intron must be transcribed to be effective. A preferred position for an intron is 3′ to the transcription start site, and 5′ to the polyA transcription termination sequence. For cDNA transgenes, an intron is placed on one side or the other (i.e., 5′ or 3′) of the transgene coding sequence. Any intron from any source, including any viral, prokaryotic and eukaryotic (plant or animal) organisms, may be used to express the polypeptide, provided that it is compatible with the host cell(s) into which it is inserted. Also included herein are synthetic introns. Optionally, more than one intron may be used in the vector.

Exemplary vectors for recombinant expression are those that are compatible with bacterial, insect, and mammalian host cells. Such vectors include, inter alia, pCRII (Invitrogen Company, San Diego, Calif.), pBSII (Stratagene Company, La Jolla, Calif.), and pETL (BlueBacII; Invitrogen).

After the vector has been constructed and a nucleic acid has been inserted into the proper site of the vector, the completed vector may be inserted into a suitable host cell for amplification and/or polypeptide expression. Commonly used include: Prokaryotic cells such as gram negative or gram positive bacteria, i.e., any strain of E. coli, Bacillus, Streptomyces, Saccharomyces, Salmonella, and the like; eukaryotic cells such as CHO (Chinese hamster ovary) cells; human kidney 293 cells; COS-7 cells; insect cells such as Sf4, Sf5, Sf9, and Sf21 and High 5 (all from the Invitrogen Company, San Diego, Calif.); plant cells and various yeast cells such as Saccharomyces and Pichia. Any transformable or transfectable cell or cell line derived from any organism such as bacteria, yeast, fungi, monocot and dicot plants, plant cells, and animals are suitable.

Insertion (also referred to as “transformation” or “transfection”) of the vector into the selected host cell may be accomplished using such methods as calcium chloride, electroporation, microinjection, lipofection or the DEAE-dextran method. The method selected will in part be a function of the type of host cell to be used. These methods and other suitable methods are well known to the skilled artisan, and are set forth, for example, in Sambrook, et al., supra. The host cells containing the vector (i.e., transformed or transfected) may be cultured using standard media well known to the skilled artisan. The media will usually contain all nutrients necessary for the growth and survival of the cells. Suitable media for culturing E. coli cells are for example, Luria Broth (LB) and/or Terrific Broth (TB). Suitable media for culturing eukaryotic cells are RPMI 1640, MEM, DMEM, all of which may be supplemented with serum and/or growth factors as required by the particular cell line being cultured. A suitable medium for insect cultures is Grace's medium supplemented with yeastolate, lactalbumin hydrolysate, and/or fetal calf serum as necessary.

Typically, an antibiotic or other compound useful for selective growth of the transformed cells only is added as a supplement to the media. The compound to be used will be dictated by the selectable marker element present on the plasmid with which the host cell was transformed. For example, where the selectable marker element is kanamycin resistance, the compound added to the culture medium will be kanamycin.

The amount of polypeptide produced in the host cell can be evaluated using standard methods known in the art. Such methods include, without limitation, Western blot analysis, SDS-polyacrylamide gel electrophoresis, non-denaturing gel electrophoresis, HPLC separation, immunoprecipitation, and/or binding assays.

If the polypeptide has been designed to be secreted from the host cells, the majority of polypeptide will likely be found in the cell culture medium. If, however, the polypeptide is not secreted from the host cells, it will be present in the cytoplasm (for eukaryotic, gram positive bacteria, and insect host cells) or in the periplasm (for gram negative bacteria host cells).

For intracellular polypeptides, the host cells are first disrupted mechanically or osmotically to release the cytoplasmic contents into a buffered solution. The polypeptide is then isolated from this solution.

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type. A cell line substantially enriched in such cells can be then isolated to provide a stable cell line.

A particularly preferred method of high-yield production of a recombinant polypeptide of the present invention is through the use of dihydrofolate reductase (DHFR) amplification in DHFR-deficient CHO cells, by the use of successively increasing levels of methotrexate as described in U.S. Pat. No. 4,889,803. The polypeptide obtained may be in a glycosylated form.

Purification of the polypeptide from solution can be accomplished using a variety of techniques. If the polypeptide has been synthesized such that it contains a tag such as hexahistidine or other small peptide at either its carboxyl or amino terminus, it may essentially be purified in a one-step process by passing the solution through an affinity column where the column matrix has a high affinity for the tag or for the polypeptide directly (i.e., a monoclonal antibody specifically recognizing the polypeptide). For example, polyhistidine binds with great affinity and specificity to nickel, thus an affinity column of nickel (such as the Qiagen nickel columns) can be used for purification of the His-tagged polypeptide. (See, for example, Ausubel, et al., eds., “Current Protocols In Molecular Biology,” Section 10.11.8, John Wiley & Sons, New York (1993)).

The strong affinity a ligand for its receptor permits affinity purification of ligand binding molecules, and ligand binding molecules using an affinity matrix comprising a complementary binding partner. Affinity chromatography may be employed, e.g., using either natural binding partners (e.g., a ligand when purifying a ligand binding molecule with affinity for the same) or antibodies generated using standard procedures (e.g., immunizing a mouse, rabbit or other animal with an appropriate polypeptide). The peptides of the present invention may be used to generate such antibodies. Known antibodies or antibodies to known growth factor receptors may be employed when they share an epitope with a targeted ligand binding molecule.

In addition, other well-known procedures for purification can be used. Such procedures include, without limitation, ion exchange chromatography, molecular sieve chromatography, HPLC, native gel electrophoresis in combination with gel elution, and preparative isoelectric focusing (“Isoprime” machine/technique, Hoefer Scientific). In some cases, two or more of these techniques may be combined to achieve increased purity. Preferred methods for purification include polyhistidine tagging and ion exchange chromatography in combination with preparative isoelectric focusing.

Polypeptide found in the periplasmic space of the bacteria or the cytoplasm of eukaryotic cells, the contents of the periplasm or cytoplasm, including inclusion bodies (bacteria) if the processed polypeptide has formed such complexes, can be extracted from the host cell using any standard technique known to the skilled artisan. For example, the host cells can be lysed to release the contents of the periplasm by French press, homogenization, and/or sonication. The homogenate can then be centrifuged.

If the polypeptide has formed inclusion bodies in the periplasm, the inclusion bodies can often bind to the inner and/or outer cellular membranes and thus will be found primarily in the pellet material after centrifugation. The pellet material can then be treated with a chaotropic agent such as guanidine or urea to release, break apart, and solubilize the inclusion bodies. The solubilized polypeptide can then be analyzed using gel electrophoresis, immunoprecipitation or the like. If it is desired to isolate the polypeptide, isolation may be accomplished using standard methods such as those set forth below and in Marchston, et al., Meth. Enz., 182:264-275 (1990).

### Gene Therapy

In some embodiments, polynucleotides of the invention further comprise additional sequences to facilitate the gene therapy. In one embodiment, a “naked” transgene encoding a ligand binding molecule described herein (i.e. a transgene without a viral, liposomal, or other vector to facilitate transfection) is employed for gene therapy.

Vectors also are useful for “gene therapy” treatment regimens, wherein a polynucleotide that encodes a ligand binding polypeptide or molecule is introduced into a subject in need of inhibition of neovascularization, in a form that causes cells in the subject to express the ligand binding molecule of the invention in vivo. Gene therapy aspects that are described in U.S. Patent Publication No. 2002/0151680 and WO 01/62942 both of which are incorporated herein by reference, also are applicable herein.

Any suitable vector may be used to introduce a polynucleotide that encodes a ligand binding molecule described herein, into the host. Exemplary vectors that have been described in the literature include replication deficient retroviral vectors, including but not limited to lentivirus vectors (Kim et al., J. Virol., 72(1): 811-816,1998; Kingsman & Johnson, Scrip Magazine, October 1998, pp. 43-46); adeno-associated viral (AAV) vectors (U.S. Pat. Nos. 5,474,935I; 5,139,941; 5,622,856; 5,658,776; 5,773,289; 5,789,390; 5,834,441; 5,863,541; 5,851,521; 5,252,479; Gnatenko et al., J. Invest. Med., 45: 87-98, 1997); adenoviral (AV) vectors (U.S. Pat. Nos. 5,792,453; 5,824,544; 5,707,618; 5,693,509; 5,670,488; 5,585,362; Quantin et al., Proc. Natl. Acad. Sci. USA, 89: 2581-2584, 1992; Stratford Perricadet et al., J. Clin. Invest., 90:626-630, 1992; and Rosenfeld et al., Cell, 68: 143-155, 1992); an adenoviral adeno-associated viral chimeric (U.S. Pat. No. 5,856,152) or a vaccinia viral or a herpesviral (U.S. Pat. Nos. 5,879,934; 5,849,571; 5,830,727; 5,661,033; 5,328,688); Lipofectin mediated gene transfer (BRL); liposomal vectors (U.S. Pat. No. 5,631,237, Liposomes comprising Sendai virus proteins); and combinations thereof. All of the foregoing documents are incorporated herein by reference in their entireties.

Other non-viral delivery mechanisms contemplated include, but are not limited to, calcium phosphate precipitation (Graham and Van Der Eb, Virology, 52:456-467, 1973; Chen and Okayama, Mol. Cell Biol., 7:2745-2752, 1987; Rippe et al., Mol. Cell Biol., 10:689-695, 1990) DEAE-dextran (Gopal, Mol. Cell Biol., 5:1188-1190, 1985), electroporation (Tur-Kaspa et al., Mol. Cell Biol., 6:716-718, 1986; Potter et al., Proc. Nat. Acad. Sci. USA, 81:7161-7165, 1984), direct microinjection (Harland and Weintraub, J. Cell Biol., 101:1094-1099, 1985.), DNA-loaded liposomes (Nicolau and Sene, Biochim. Biophys. Acta, 721:185-190, 1982; Fraley et al., Proc. Natl. Acad. Sci. USA, 76:3348-3352, 1979; Felgner, Sci Am. 276(6):102-6, 1997; Felgner, Hum Gene Ther. 7(15):1791-3, 1996), cell sonication (Fechheimer et al., Proc. Natl. Acad. Sci. USA, 84:8463-8467, 1987), gene bombardment using high velocity microprojectiles (Yang et al., Proc. Natl. Acad. Sci USA, 87:9568-9572, 1990), and receptor-mediated transfection (Wu and Wu, J. Biol. Chem., 262:4429-4432, 1987; Wu and Wu, Biochemistry, 27:887-892, 1988; Wu and Wu, Adv. Drug Delivery Rev., 12:159-167, 1993).

The expression construct (or indeed a ligand binding molecule described herein) may be entrapped in a liposome. Liposomes are vesicular structures characterized by a phospholipid bilayer membrane and an inner aqueous medium. Multi-lamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, In: Liver diseases, targeted diagnosis and therapy using specific receptors and ligands, Wu G, Wu C ed., New York: Marcel Dekker, pp. 87-104, 1991). The addition of DNA to cationic liposomes causes a topological transition from liposomes to optically birefringent liquid-crystalline condensed globules (Radler et al., Science, 275(5301):810-4, 1997). These DNA-lipid complexes are potential non-viral vectors for use in gene therapy and delivery.

Liposome-mediated nucleic acid delivery and expression of foreign DNA in vitro has been successful. Also contemplated in the present invention are various commercial approaches involving “lipofection” technology. In certain embodiments of the invention, the liposome may be complexed with a hemagglutinating virus (HVJ). This has been shown to facilitate fusion with the cell membrane and promote cell entry of liposome-encapsulated DNA (Kaneda et al., Science, 243:375-378, 1989). In other embodiments, the liposome may be complexed or employed in conjunction with nuclear nonhistone chromosomal proteins (HMG-1) (Kato et al., J. Biol. Chem., 266:3361-3364, 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., Nature, 327:70-73, 1987). Several devices for accelerating small particles have been developed. One such device relies on a high voltage discharge to generate an electrical current, which in turn provides the motive force (Yang et al., Proc. Natl. Acad. Sci USA, 87:9568-9572, 1990). The microprojectiles used have consisted of biologically inert substances such as tungsten or gold beads.

In embodiments employing a viral vector, preferred polynucleotides still include a suitable promoter and polyadenylation sequence as described above. Moreover, it will be readily apparent that, in these embodiments, the polynucleotide further includes vector polynucleotide sequences (e.g., adenoviral polynucleotide sequences) operably connected to the sequence encoding a polypeptide of the invention.

### Therapeutic Uses of the Ligand Binding Molecules

The ligand binding polypeptides and molecules described herein, and the polynucleotides and vectors that encode them, are useful for inhibiting cellular processes that are mediated through endothelial growth factors inducing signal transduction through VEGFR-2 or VEGFR-3, and have indications for prophylaxis or therapy of disorders associated with aberrant angiogenesis and/or lymphangiogenesis (e.g., various ocular disorders and cancer) that is stimulated by the actions of such growth factors on these receptors. The ligand binding polypeptides and molecules described herein, and the polynucleotides and vectors that encode them, are therapeutically useful for treating or preventing any disease of condition which is improved, ameliorated, inhibited or prevented by the removal, inhibition or reduction of VEGF-C and/or VEGF-D. A non-exhaustive list of specific conditions improved by inhibition or reduction of VEGF-C and/or VEGF-D (and in particular at least VEGF-C) include: clinical conditions that are characterized by excessive vascular endothelial cell proliferation, vascular permeability, edema or inflammation such as brain edema associated with injury, stroke or tumor; edema associated with inflammatory disorders such as psoriasis or arthritis, including rheumatoid arthritis; asthma; generalized edema associated with burns; ascites and pleural effusion associated with tumors, inflammation or trauma; chronic airway inflammation; capillary leak syndrome; sepsis; kidney disease associated with increased leakage of protein; and eye disorders such as age related macular degeneration and diabetic retinopathy.

Although for brevity many of the methods are described below with respect to compositions comprising a ligand binding molecule, it should be understood that practice of the invention with any of the constructs described herein (ligand binding polypeptides, molecules, and constructs, and polynucleotides that encode them, dimers and other multimers, etc.) is contemplated.

An exemplary therapeutic use is a method of inhibiting neovascularization in a subject in need thereof comprising administering to the subject a composition comprising a ligand binding molecule described herein, in an amount effective to inhibit neovascularization in the subject.

In some embodiments, the neovascularization comprises choroidal retinal neovascularization. In some embodiments, the neovascularization is tumor neovascularization that occurs in malignant cancers and other tumors.

In another aspect, described herein is a method of prophylaxis or therapy for an ocular disorder associated with neovascularization comprising administering to a subject in need of prophylaxis or therapy for the ocular disorder a composition comprising a ligand binding molecule described herein.

In another aspect, described herein is a method of prophylaxis or therapy for an ocular disorder which results in retinal edema comprising administering to a subject in need of prophylaxis or therapy for the ocular disorder or disease a composition comprising a ligand binding molecule described herein.

Examples of ocular disorders which may be treated include choroidal neovascularization, diabetic macular edema, age-related macular degeneration, proliferative diabetic retinopathy, retinal vein occlusion and corneal neovascularization/transplant rejection. Preferably, the amount of the ligand binding molecule employed is effective to inhibit the binding of VEGF-C and/or VEGF-D ligand to VEGFR-3 (and preferably also to VEGFR-2) or the stimulatory effect of VEGF-C and/or VEGF-D on VEGFR-3 (and preferably also VEGFR-2).

In one embodiment, the ocular disorder is age-related macular degeneration. Examples of age-related macular degeneration are non-neovascular (also known as “Dry”) and neovascular (also known as “Wet”) macular degeneration. In a preferred embodiment, the ocular disorder is wet age-related macular degeneration. Treating or preventing wet age-related macular degeneration also encompasses treating or preventing choroidal neovascularization or pigment epithelial detachment.

In one embodiment, the ocular disorder is polypoidal choroidal vasculopathy. Polypoidal choroidal vasculopathy is characterized by a lesion from an inner choroidal vascular network of vessels ending in an aneurysmal bulge or outward projection (Ciardella et al. (2004) Surv Ophthalmol. 49:25-37).

In one embodiment, the ocular disorder is a condition associated with choroidal neovascularization. Examples of conditions associated with choroidal neovascularization include a degenerative, inflammatory, traumatic or idiopathic condition. Treating or preventing a degenerative disorder associated with choroidal neovascularization also encompasses treating or preventing a heredodegenerative disorder. Examples of heredodegenerative disorders include vitelliform macular dystrophy, fundus flavimaculatus and optic nerve head drusen. Examples of degenerative conditions associated with choroidal neovascularization include myopic degeneration or angioid streaks. Treating or preventing an inflammatory disorder associated with choroidal neovascularization also encompasses treating or preventing ocular histoplasmosis syndrome, multifocal choroiditis, serpininous choroiditis, toxoplasmosis, toxocariasis, rubella, Vogt-Koyanagi-Harada syndrome, Behcet syndrome or sympathetic ophthalmia. Treating or preventing a traumatic disorder associated with choroidal neovascularization also encompasses treating or preventing choroidal rupture or a traumatic condition caused by intense photocoagulation.

In one embodiment, the ocular disorder is hypertensive retinopathy.

In one embodiment, the ocular disorder is diabetic retinopathy. Diabetic retinopathy can be non-proliferative or proliferative diabetic retinopathy. Examples of non-proliferative diabetic retinopathy include macular edema and macular ischemia.

In one embodiment, the ocular disorder is sickle cell retinopathy.

In one embodiment, the ocular disorder is a condition associated with peripheral retinal neovascularization. Examples of conditions associated with peripheral retinal neovascularization include ischemic vascular disease, inflammatory disease with possible ischemia, incontinentia pigmenti, retinitis pigmentosa, retinoschisis or chronic retinal detachment.

Examples of ischemic vascular disease include proliferative diabetic retinopathy, branch retinal vein occlusion, branch retinal arteriolar occlusion, carotid cavernous fistula, sickling hemoglobinopathy, non-sickling hemoglobinopathy, IRVAN syndrome (retinal vasculitic disorder characterized by idiopathic retinal vasculitis, an aneurysm, and neuroretinitis), retinal embolization, retinopathy of prematurity, familial exudative vitreoretinopathy, hyperviscosity syndrome, aortic arch syndrome or Eales disease. Examples of sickling hemoglobinopathy include SS hemoglobinopathy and SC hemoglobinopathy. Examples of non-sickling hemoglobinopathy include AC hemoglobinopathy and AS hemoglobinopathy. Examples of hyperviscosity syndrome include leukemia, Waldenstrom macroglobulinemia, multiple myeloma, polycythemia or myeloproliferative disorder.

Treating or preventing an inflammatory disease with possible ischemia also encompasses treating or preventing retinal vasculitis associated with systemic disease, retinal vasculitis associated with an infectious agent, uveitis or birdshot retinopathy. Examples of systemic diseases include systemic lupus erythematosis, Behcet's disease, inflammatory bowel disease, sarcoidosis, multiple sclerosis, Wegener's granulomatosis and polyarteritis nodosa. Examples of infectious agents include a bacterial agent that is the causative agent for syphilis, tuberculosis, Lyme disease or cat-scratch disease, a virus such as herpesvirus, or a parasite such as Toxocara canis or Toxoplasma gondii. Examples of uveitis include pars planitis or Fuchs uveitis syndrome.

In one embodiment, the ocular disorder is retinopathy of prematurity. Retinopathy of prematurity can result from abnormal growth of blood vessels in the vascular bed supporting the developing retina (Pollan C (2009) Neonatal Netw. 28:93-101).

In one embodiment, the ocular disorder is venous occlusive disease. Examples of venous occlusive disease include branch retinal vein occlusion and central retinal vein occlusion. A branch retinal vein occlusion can be a blockage of the portion of the circulation that drains the retina of blood. The blockage can cause back-up pressure in the capillaries, which can lead to hemorrhages and also to leakage of fluid and other constituents of blood.

In one embodiment, the ocular disorder is arterial occlusive disease. Examples of arterial occlusive disease include branch retinal artery occlusion, central retinal artery occlusion or ocular ischemic syndrome. A branch retinal artery occlusion (BRAO) can occur when one of the branches of the arterial supply to the retina becomes occluded.

In one embodiment, the ocular disorder is central serous chorioretinopathy (CSC). In one embodiment, CSC is characterized by leakage of fluid in the central macula.

In one embodiment, the ocular disorder is cystoid macular edema (CME). In one embodiment, CME affects the central retina or macula. In another embodiment, CME occurs after cataract surgery.

In one embodiment, the ocular disorder is retinal telangiectasia. In one embodiment, retinal telangiectasia is characterized by dilation and tortuosity of retinal vessels and formation of multiple aneurysms. Idiopathic JXT, Leber's miliary aneurysms, and Coats' disease are three types of retinal telangiectasias.

In one embodiment, the ocular disorder is arterial macroaneurysm.

In one embodiment, the ocular disorder is retinal angiomatosis. In one embodiment, retinal angiomatosis occurs when the ocular vessels form multiple angiomas.

In one embodiment, the ocular disorder is radiation-induced retinopathy (RIRP). In one embodiment, RIRP may display symptoms such as macular edema and nonproliferative and proliferative retinopathy.

In one embodiment, the ocular disorder is rubeosis iridis. In another embodiment, rubeosis iridis results in the formation of neovascular glaucoma. In another embodiment, rubeosis iridis is caused by diabetic retinopathy, central retinal vein occlusion, ocular ischemic syndrome, or chronic retinal detachment.

In one embodiment, the ocular disorder is a neoplasm. Examples of neoplams include an eyelid tumor, a conjunctival tumor, a choroidal tumor, an iris tumor, an optic nerve tumor, a retinal tumor, an infiltrative intraocular tumor or an orbital tumor. Examples of an eyelid tumor include basal cell carcinoma, squamous carcinoma, sebaceous carcinoma, malignant melanoma, capillary hemangioma, hydrocystoma, nevus or seborrheic keratosis. Examples of a conjunctival tumor include conjunctival Kaposi's sarcoma, squamous carcinoma, intraepithelial neoplasia of the conjunctiva, epibular dermoid, lymphoma of the conjunctiva, melanoma, pingueculum, or pterygium. Examples of a choroidal tumor include choroidal nevus, choroidal hemangioma, metastatic choroidal tumor, choroidal osteoma, choroidal melanoma, ciliary body melanoma or nevus of Ota. Examples of an iris tumor include anterior uveal metastasis, iris cyst, iris melanocytoma, iris melanoma, or pearl cyst of the iris. Examples of an optic nerve tumor include optic nerve melanocytoma, optic nerve sheath meningioma, choroidal melanoma affecting the optic nerve, or circumpapillary metastasis with optic neuropathy. Examples of a retinal tumor include retinal pigment epithelial (RPE) hypertrophy, RPE adenoma, RPE carcinoma, retinoblastoma, hamartoma of the RPE, or von Hippel angioma. Examples of an infiltrative intraocular tumor include chronic lymphocytic leukemia, infiltrative choroidopathy, or intraocular lymphoma. Examples of an orbital tumor include adenoid cystic carcinoma of the lacrimal gland, cavernous hemangioma of the orbit, lymphangioma of the orbit, orbital mucocele, orbital pseudotumor, orbital rhabdomyosarcoma, periocular hemangioma of childhood, or sclerosing orbital psuedotumor.

In a further aspect, the invention features a method of treating an eye injury, comprising locally administering an effective amount of a ligand binding molecule described herein to a subject in need thereof, such that the eye injury is ameliorated or improved. Preferably, the eye injury is a corneal injury or conjunctival injury and the method of treatment reduces angiogenesis and inflammation associated with the eye injury. In some embodiments, the method is useful to treat acute and sub-acute corneal injury or conjunctival injury. Acute corneal injury may be treated within 24 hours of occurrence, and includes corneal injury or conjunctival injury caused by a penetrating object, a foreign body, or a chemical or burn injury. A sub-acute injury may be treated up to two weeks post-injury and may include the above listed injuries as well as infectious etiologies. In some embodiments, the eye injury is caused by trauma, e.g., surgical injuries, chemical burn, corneal transplant, infectious or inflammatory diseases.

Length of treatment will vary according to the injury, but treatment duration may be short, e.g., up to one month, and may include a 3-6 month observation period, during which retreatment may be provided. Administration may also include a second agent, such as an immunosuppressive agent, for example, one or more of a corticosteroid, dexamethasone, or cyclosporin A. Local administration includes, for example, administration of the ligand binding molecule in eye drops applied to the eye, or subconjunctival injection to the eye.

In a further aspect, described herein is a method of healing an eye injury, comprising locally administering an effective amount of a ligand binding molecule described herein to a subject in need thereof, such that the eye injury heals.

In a further aspect, described herein is a method of reducing or ameliorating angiogenesis associated with an eye injury, comprising locally administering an effective amount of a ligand binding molecule described herein to a subject in need thereof, such that the angiogenesis associated with the eye injury is reduced or ameliorated.

In a further aspect, described herein is a method of reducing or ameliorating inflammation associated with an eye injury, comprising locally administering an effective amount of a ligand binding molecule described herein to a subject in need thereof, such that the inflammation associated with the eye injury is reduced or ameliorated.

In a further aspect, described herein is a method of administering a ligand binding molecule of the present invention for treatment of angiogenesis and/or inflammation associated with eye injury or infection, comprising local administration by eye drops comprising a ligand binding molecule described herein, or subconjunctival administration by injection or implantation.

In a further aspect, the described herein is a method of extending corneal graft survival following corneal transplantation in a patient by administering to the patient an effective amount of a pharmaceutical composition containing a ligand binding molecule described herein (whereby angiogenesis and/or lymphangiogenesis is suppressed in the cornea of the patient).

Dose response studies permit accurate determination of a proper quantity of ligand binding molecule to employ. Effective quantities can be estimated, for example, from measurements of the binding affinity of a polypeptide for a target receptor, of the quantity of receptor present on target cells, of the expected dilution volume (e.g., patient weight and blood volume for in vivo embodiments), and of polypeptide clearance rates. For example, existing literature regarding dosing of known VEGF-C antibodies known also provides guidance for dosing of the ligand binding molecules described herein. Literature describing dosing of Aflibercept (Regeneron), a ligand trap based on VEGFR-1/VEGFR-2, also may be used to provide guidance for dosing of therapeutic molecules described herein.

In some embodiments, when being administered by intravitreal injection, the ligand binding molecule is administered in a concentration of about 2 mg to about 4 mg per eye (or about 1 mg to about 3 mg, or about 1 mg to about 4 mg, or about 3 mg to about 4 mg, or about 1 mg to about 2 mg per eye). In some embodiments, the ligand binding molecule is administered in a concentration of about 1 mg, or about 2 mg, or about 3 mg, or about 4 mg, or about 5 mg, or about 6 mg per eye. The ligand binding molecule, in some embodiments, is present in any of the concentrations listed above in a volume of 10 μl, 15 μl, 20 μl, 25 μl, 30 μl, 35 μl, 40 μl, 45 μl, 50 μl, 60 μl, 70 μl, 80 μl, 90 μl, 95 μl or 100 μl. In some embodiments, the ligand binding molecule is administered at a concentration of about 2-4 mg/50 μl.

The ligand binding molecule described herein can be administered purely as a prophylactic treatment to prevent neovascularization in subjects at risk for developing an ocular disease associated with neovascularization (e.g., diabetic retinopathy, macular degeneration) , or as a therapeutic treatment to subjects afflicted the ocular disease, for the purpose of inhibiting neovascularization in the eye of a subject in need thereof.

Subjects who are at risk of developing diabetic retinopathy or macular degeneration include subjects over the age of fifty; subjects afflicted with rheumatoid arthritis, subjects with diabetes, subjects with thyroid abnormalities, subjects with asthma, subjects with cataracts, subjects with glaucoma, subjects with lupus, subjects with high blood pressure and subjects with retinal detachment. Other risk factors include genetics, diet, smoking and sublight exposure.

In some embodiments, described herein is a method of selecting a therapeutic regimen for a subject in need thereof comprising screening a subject for one or more symptoms of an ocular disorder associated with retinal neovascularization and prescribing for the subject administration of a composition comprising a ligand binding molecule described herein. In another embodiment, described herein is a method of treating a subject affected with an ocular disorder associated with retinal neovascularization comprising identifying a subject as having one or more symptoms of the ocular disorder and administering a composition comprising a ligand binding molecule to the subject. Symptoms associated with an ocular disorder associated with retinal neovascularization include, but are not limited to, blurred vision and slow vision loss over time, tiny particles drifting inside the eye, shadows or missing areas of vision, distorted vision and night blindness.

In some embodiments, the methods described herein further comprise prescribing (or administering) a standard of care regimen for the treatment of dry eye disease. In the context of methods described herein, “standard of care” refers to a treatment that is generally accepted by clinicians for a certain type of patient diagnosed with a type of illness. For diabetic retinopathy and macular degeneration, for example, an aspect of the invention is to improve standard of care therapy with co-therapy with a ligand binding molecule described herein that inhibit retinal neovascularization. Exemplary standard of care therapeutic for diabetic retinopathy and macular degeneration include, but are not limited to, eyelid hygiene, topical antibiotics (including, but not limited to erythromycin or bacitracin ointments), oral tetracyclines (tetracycline, doxycycline, or minocycline), anti-inflammatory compounds (including, but not limited to, cyclosporine), corticosteroids, laser photocoagulation and photodynamic therapy.

Also contemplated are methods of treating a mammalian subject with an ocular disorder associated with retinal neovascularization that is hypo-responsive to a standard of care regimen for the treatment of the ocular disorder comprising administering a ligand binding molecule to the subject in an amount effective to treat the disorder.

The mammalian subject is preferably a human subject. Practice of methods of the invention in other mammalian subjects, especially mammals that are conventionally used as models for demonstrating therapeutic efficacy in humans (e.g., primate, porcine, canine, or rabbit animals), is also contemplated.

### Combination Therapies and Additional Active Agents

Combination therapy and prophylactic embodiments of the invention include products and methods. Exemplary compounds that may be administered in combination with one or more of the ligand binding molecules described herein include, but are not limited to, the compounds provided below in Table 2.

| | | |
|---|---|---|
| Product | Target or Mechanism of Action | Comments |

VEGF-A Inhibitors

| | | |
|---|---|---|
| KH902 | VEGF-A inhibitor | VEGF-Receptor-Fc Recombinant |
| | | fusion protein with ligand binding |
| | | domain taken from VEGFR-1 and |
| | | VEGFR-2 that binds all VEGF-A |
| | | isoforms and PIGF but not VEGF-C |
| | | or -D |
| VEGF-A DARPin | VEGF-A inhibitor | Derived from ankyrin protein with |
| (AGN-150998) | | selective binding to VEGF-A and |
| | | not other members of the VEGF |
| | | family. |
| ESBA1008 | Single chain antibody fragment to | |
| | VEGF-A | |
| Ranibizumab | Monoclonal antibody fragment | Derived from the same parent |
| (Lucentis ™) | (Fab) | mouse antibody as bevacizumab |
| | | (Avastin ™) |

Anti-Pericyte (PDGF-B Inhibitors)

| | | |
|---|---|---|
| E10030 | Anti-PDGF aptamer | Targets pericyte mediated |
| (Fovista ™) | | resistance to anti-VEGF-A therapy. |

Multi-Targeted Kinase Inhibitors

| | | |
|---|---|---|
| Vatalanib | Tyrosine kinase inhibitor | |
| (PTK787/PTK/ZK | | |
| AL-39324 | Tyrosine kinase inhibitor | Injectable. |
| Pazopanib | Tyrosine kinase inhibitor | TKI of VEGFR-1, VEGFR-2, |
| | | VEGFR-3, PDGFR-a/b and cKit. |
| | | Topical eye drop application. |
| TG100801 | Tyrosine kinase inhibitor | Prodrug inhibits VEGF, PDGF, FGF |
| | | receptors and Src family of kinases. |
| | | Topical administration. |
| Squalamine | Small molecule aminosterol binds | Binds calmodulin and prevents |
| | calmodulin | modulation of VEGF, PDGF and |
| | | bFGF. |

mTOR Pathway Inhibitors

| | | |
|---|---|---|
| Sirolimus (DE-109) | mTOR inhibitor | Broad acting anti-proliferative and |
| | | immune suppressive agent. |
| Sirolimus | mTOR inhibitor | |
| PF-655 | Synthetic siRNA to RTP801 | Stress induced mTOR inhibitor that |
| (REDD14P) | (mTOR regulator) | stabilises TSC1-TSC2 inhibitory |
| | | complex and enhances oxidative |
| | | stress-dependent cell death. |
| Palomid529 | Small molecule TORC1/TORC2 | |
| | inhibitor (mTOR pathway) | |

Vascular Disrupting Agents

| | |
|---|---|
| Zybrestat | VDA (vascular disrupting agent) |
| | and cadherin 5 inhibitor |
| Fosbretabulin | Vascular disrupting agent (VDA) |
| (combretastatin | |
| A4 phosphate) | |

Anti-Inflammatory Agents
Corticosteroids

| | |
|---|---|
| Posurdex/SK- | Corticosteroid and VEGF-A |
| 0503 | inhibitor |
| Iluvien | Corticosteroid (intravitreal insert) |
| (fluocinolone | |
| acetonide) | |
| IBI-20089 | Slow release triamcinolone |

Complement Inhibitors

| | | |
|---|---|---|
| LFG316 | Anti-C5 (complement pathway) | Selectively targets inflammation |
| | | associated with AMD |
| ARC1905 | Anti-C5 aptamer | |
| AL-78898A | Anti-C3 cyclic peptide | Targets C3 in the complement |
| (POT-4) | | pathway. |

‘Other’ Anti-Inflammatory Agents

| | |
|---|---|
| Humira | Anti-TNF mAb |
| (adalimumab) | |

Miscellaneous Targeted Agents

| | | |
|---|---|---|
| iSONEP | Anti-S1P mAb | mAb targets the lipid sphingosine-1- |
| | | phosphate |
| Ocriplasmin | Truncated form of Human serine | Approved for the treatment of |
| | protease plasmin | symptomatic vitreomacular |
| | | adhesion |
| Volociximab | Chimeric Ab to a5b1 integrin | Blocks binding of a5b1 integrin to |
| | | fibronectin involved in vascular |
| | | stabilisation |
| hl-con1 | Anti-Tissue Factor | Chimeric, IgG-like homodimeric |
| | | protein composed of a mutant factor |
| | | V11a domain fused to an effector |
| | | region (IgG Fc). Mutant fV11 binds |
| | | to tissue factor which is expressed |
| | | on the luminal surface of pathologic |
| | | cells including AMD lesions, |
| | | triggering immune destruction of h1- |
| | | con1 targeted cells via effector |
| | | functions |
| ORA102 | Target unknown. | |

Gene Therapy

| | | |
|---|---|---|
| rAAV.sFlt-1 | Adenovrial gene delivery of | Sub-retinally delivered gene |
| | soluble form of VEGFR-1. | therapy. ‘Traps’ VEGFR-1 ligands |
| | | only (VEGF-A, VEGF-B, PIGF). |
| adPEDF | Adenoviral gene delivery of | PEDF is anti-angiogenic (inhibits |
| | Pigment epithelium derived | VEGF induced proliferation, EC |
| | factor (PEDF) | migration and permeability). |
| RetinoStat | Lentiviral delivery of angiostatin | Angiostatin (fragment of plasmin) |
| | & endostatin | and endostatin (C-term fragement |
| | | of Type XVIII collagen) are |
| | | endogenous inhibitors of |
| | | angiogenesis. |
| AAV2-sFLT01 | Adenoviral gene delivery of | Intravitreally delivered gene |
| | soluble form of VEGFR-1 | therapy. ‘Traps’ VEGFR-1 ligands |
| | | only (VEGF-A, VEGF-B, PIGF) |

Antisense & siRNA

| | | |
|---|---|---|
| GS-101 | Antisense targeting IRS-1 | Topical application of antisense to |
| | | Insulin-Receptor-Substrate-1 |
| Bevasiranib | siRNA targeting VEGF | |
| AGN211745 | siRNA targeting VEGFR-1 | |

The ligand binding molecules may be administered in combination with one or more additional active compounds or therapies, including a second receptor trap molecule, a cytotoxic agent, surgery, catheter devices and radiation. Exemplary combination products include two or more agents formulated as a single composition or packaged together in separate compositions, e.g., as a unit dose package or kit. Exemplary combination methods include prescribing for administration, or administration of two or more agents simultaneously or concurrently or at staggered times (i.e., sequentially).

The term “cytotoxic agent” as used herein refers to a substance that inhibits or prevents the function of cells and/or causes destruction of cells. The term is intended to include radioactive isotopes (e.g. I¹³¹, I¹²⁵, Y⁹⁰ and Re¹⁸⁶), chemotherapeutic agents, and toxins such as enzymatically active toxins of bacterial, fungal, plant or animal origin, or fragments thereof. A “chemotherapeutic agent” is a chemical compound useful in the treatment of cancer. Examples of chemotherapeutic agents include alkylating agents such as thiotepa and cyclosphosphamide (Cytoxan®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estrainustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (Taxol®, Bristol-Myers Squibb Oncology, Princeton, N.J.) and docetaxel (Taxotere®; Aventis Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; and pharmaceutically acceptable salts, acids or derivatives of any of the above.

A “growth inhibitory agent” when used herein refers to a compound or composition which inhibits growth of a cell, especially a cancer cell either in vitro or in vivo. Examples of growth inhibitory agents include agents that block cell cycle progression (at a place other than S phase), such as agents that induce G 1 arrest and M-phase arrest. Classical M-phase blockers include the vincas (vincristine and vinblastine), Taxol ®, and topo II inhibitors such as doxorubicin, epirubicin, daunorubicin, etoposide, and bleomycin. Those agents that arrest G 1 also spill over into S-phase arrest, for example, DNA alkylating agents such as tamoxifen, prednisone, dacarbazine, mechlorethamine, cisplatin, methotrexate, 5-fluorouracil, and ara-C.

### VEGF-A (VEGF) Inhibitor Products

In some embodiments, methods described herein optionally comprise administering a therapeutic active to inhibit VEGF-A binding to one or more of its receptors, especially VEGFR-2. A VEGF-A inhibitor product may be administered in combination with one or more of the ligand binding molecules described herein. In some embodiments, the VEGF-A inhibitor product and the ligand binding molecule are co-administered in a single composition. In other embodiments, the VEGF-A inhibitor product is administered as a separate composition from the ligand binding molecule.

In one embodiment, the VEGF-A inhibitor product is selected from ranibizumab, bevacizumab, aflibercept, KH902 VEGF receptor-Fc fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-hydroxyisovalerylshikonin, EYE001, ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, or G6-31 antibody, or a pharmaceutically acceptable salt thereof of any of the aforementioned.

cDNA and amino acid sequences of human VEGFR-2 ECD are set forth in SEQ ID NOs: 5 and 6, respectively. The “VEGF-A inhibitor product” can be any molecule that acts with specificity to reduce VEGF-A/VEGFR-2 interactions, e.g., by blocking VEGF-A binding to VEGFR-2 or by reducing expression of VEGFR-2. The term “VEGF-A” as used herein refers to the vascular endothelial growth factor that induces angiogenesis or an angiogenic process and includes the various subtypes of VEGF that arise by, e.g., alternative splicing of the VEGF-A gene including VEGF₁₂₁, VEGF₁₆₅ and VEGF₁₈₉ induce angiogenesis or an angiogenic process. The term “VEGF” can be used to refer to a “VEGF” polypeptide or a “VEGF” encoding gene or nucleic acid.

The term “VEGF-A inhibitor product” refers to an agent that reduces, or inhibits, either partially or fully, the activity or production of VEGF-A. A VEGF-A inhibitor product can directly or indirectly reduce or inhibit the activity or production of a specific VEGF-A such as VEGF₁₆₅. Furthermore, “VEGF-A inhibitor products” include agents that act on either a VEGF-A ligand or its cognate receptor so as to reduce or inhibit a VEGF-A associated receptor signal. Examples of “VEGF-A inhibitor products” include antisense molecules, ribozymes or RNAi that target a VEGF-A nucleic acid; VEGF-A aptamers; VEGF-A antibodies; soluble VEGF receptor decoys that prevent binding of a VEGF-A to its cognate receptor; antisense molecules, ribozymes, or RNAi that target a cognate VEGF-A receptor (VEGFR-1 and/or VEGFR-2) nucleic acid; VEGFR-1 and VEGFR-2 aptamers or VEGFR-1 and VEGFR-2 antibodies; and VEGFR-1 and/or VEGFR-2 tyrosine kinase inhibitors.

The VEGF-A inhibitor can be a polypeptide comprising a soluble VEGFR-2 ECD fragment (amino acids 20-764 of SEQ ID NO: 6) that binds VEGF; a soluble VEGFR-1 ECD fragment, a soluble VEGFR-1/R2 based ligand trap, such as Af1ibercept (Regeneron); VEGFR-2 anti-sense polynucleotides or short-interfering RNA (siRNA); anti-VEGFR-2 antibodies; a VEGFR-2 inhibitor polypeptide comprising an antigen-binding fragment of an anti-VEGFR-2 antibody that inhibits binding between VEGFR-2 and VEGF; an aptamer that inhibits binding between VEGFR-2 and VEGF-A. In some variations, the VEGFR-2 based ligand trap comprises a fusion protein comprising the soluble VEGFR-2 polypeptide fragment fused to an immunoglobulin constant region fragment (Fc). In some embodiments, a VEGFR-2 polypeptide fragment is fused to alkaline phosphatase (AP). Methods for making Fc or AP fusion constructs are found in WO 02/060950, the disclosure of which is incorporated herein by reference in its entirety.

A number of VEGF-A antibodies have been described, see for example, U.S. Pat. Nos. 8,349,322; 8,236,312; 8,216,571; 8,101,177; 8092,797; 8,088,375; 8,034,905; 5,730,977; 6,342,219, 6,524,583, 6,451,764, 6,448,077, 6,416,758, 6,342,221 and PCT publications WO 96/30046, WO 97/44453, and WO 98/45331, the contents of which are incorporated by reference in their entirety. Exemplary VEGF-A antibodies include Bevacizumab (Avastin®) and Ranibizumab (Lucentis®). In some embodiments, one or more ligand binding molecules described herein are administered in combination with bevacizumab. In some embodiments, one or more ligand binding molecule described herein are administered in combination with ranibizumab.

In some embodiments, the VEGF-A inhibitor is EYE001 (previously referred to as NX1838), which is a modified, PEGylated aptamer that binds with high and specific affinity to the major soluble human VEGF isoform (see, U.S. Pat. Nos. 6,011,020; 6,051,698; and 6,147,204). The aptamer binds and inactivates VEGF in a manner similar to that of a high-affinity antibody directed towards VEGF. Another useful VEGF aptamer is EYE001 in its non-pegylated form.

In a preferred embodiment, one or more ligand binding molecules described herein are administered in combination with aflibercept (Eylea®) (Holash et al., Proc. Natl. Acad. Sci. USA, 99:11393-11398, 2002, the disclosure of which is incorporated herein by reference in its entirety.

A number of VEGFR-2 antibodies have been described, see for example, U.S. Pat. No. 6,334,339 and U.S. Patent Publication Nos. 2002/0064528, 2005/0214860, and 2005/0234225 (all of which are incorporated herein by reference in their entireties). Antibodies are useful for modulating VEGFR-2/VEGF interactions due to the ability to easily generate antibodies with relative specificity, and due to the continued improvements in technologies for adopting antibodies to human therapy. Thus, the invention contemplates use of antibodies (e.g., monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR sequences which specifically recognize a polypeptide of the invention) specific for VEGFR-2. Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86 95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779 783 (1992); Lonberg et al., Nature 368 856 859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13:65-93 (1995).

### PDGF Inhibitor Products

In some embodiments, methods described herein optionally comprise administering a therapeutic active to inhibit PDGF binding to one or more of its receptors. A PDGF Inhibitor Product inhibitor product may be administered in combination with one or more of the ligand binding molecules described herein. In some embodiments, the PDGF inhibitor product and the ligand binding molecule are co-administered in a single composition. In other embodiments, the PDGF inhibitor product is administered as a separate composition from the ligand binding molecule.

The term “PDGF” refers to a platelet-derived growth factor that regulates cell growth or division. As used herein, the term “PDGF” includes the various subtypes of PDGF including PDGF-B, PDGF-A, PDGF-C, PDGF-D, variant forms thereof and dimerized forms thereof, including PDGF-AA, PDGF-AB, PDGF-BB, PDGF-CC, and PDGF-DD. Platelet derived growth factors includes homo-or heterodimers of A-chain (PDGF-A) and B-chain (PDGF-B) that exert their action via binding to and dimerization of two related receptor tyrosine kinase platelet-derived growth factor cell surface receptors (i.e., PDGFRs), PDGFR-a and PDGFR-β. In addition, PDGF-C and PDGF-D, two additional protease-activated ligands for the PDGFR complexes, have been identified (Li et al., (2000) Nat. Cell. Biol. 2: 302-9; Bergsten et al., (2001) Nat. Cell. Biol. 3: 512-6; and Uutele et al., (2001) Circulation 103: 2242-47). Due to the different ligand binding specificities of the PDGFRs, it is known that PDGFR-α/α binds PDGF-AA, PDGF-BB, PDGF-AB, and PDGF-CC; PDGFR-β/β binds PDGF-BB and PDGF-DD; whereas PDGFR-α/β binds PDGF-AB, PDGF-BB, PDGF-CC, and PDGF-DD (Betsholtz et al., (2001) BioEssays 23: 494-507). As used herein, the term “PDGF” also refers to those members of the class of growth factors that induce DNA synthesis and mitogenesis through the binding and activation of a PDGFR on a responsive cell type. PDGFs can effect, for example: directed cell migration (chemotaxis) and cell activation; phospholipase activation; increased phosphatidylinositol turnover and prostaglandin metabolism; stimulation of both collagen and collagenase synthesis by responsive cells; alteration of cellular metabolic activities, including matrix synthesis, cytokine production, and lipoprotein uptake; induction, indirectly, of a proliferative response in cells lacking PDGF receptors; and potent vasoconstrictor activity. The term “PDGF” can be used to refer to a “PDGF” polypeptide, a “PDGF” encoding gene or nucleic acid, or a dimerized form thereof.

The term “PDGF inhibitor product” refers to an agent that reduces, or inhibits, either partially or fully, the activity or production of a PDGF. A PDGF inhibitor product can directly or indirectly reduce or inhibit the activity or production of a specific PDGF such as PDGF-B. Furthermore, “PDGF inhibitor products” include agents that act on a PDGF ligand or its cognate receptor so as to reduce or inhibit a PDGF-associated receptor signal. Examples of “PDGF inhibitor products” include antisense molecules, ribozymes or RNAi that target a PDGF nucleic acid; PDGF aptamers, PDGF antibodies to PDGF itself or its receptor, or soluble PDGF receptor decoys that prevent binding of a PDGF to its cognate receptor; antisense molecules, ribozymes or RNAi that target a cognate PDGF receptor (PDGFR) nucleic acid; PDGFR aptamers or PDGFR antibodies that bind to a cognate PDGFR receptor; and PDGFR tyrosine kinase inhibitors.

In one embodiment, the PDGF inhibitor product is selected from: a compound of Formula A, B, C, D or E as described and defined is US 2012/0100136 (the entire contents of which are herein incorporated by reference), p1B3 antibody, CDP860, IMC-3G3, 162.62 antibody, 163.31 antibody, 169.14 antibody, 169.31 antibody, αR1 antibody, 2A1E2 antibody, M4TS.11 antibody, M4TS.22 antibody, Hyb 120.1.2.1.2 antibody, Hyb 121.6.1.1.1 antibody, Hyb 127.5.7.3.1 antibody, Hyb 127.8.2.2.2 antibody, Hyb 1.6.1 antibody, Hyb 1.11.1 antibody, Hyb 1.17.1 antibody, Hyb 1.18.1 antibody, Hyb 1.19.1 antibody, Hyb 1.23.1 antibody, Hyb 1.24 antibody, Hyb 1.25 antibody, Hyb 1.29 antibody, Hyb 1.33 antibody, Hyb 1.38 antibody, Hyb 1.39 antibody, Hyb 1.40 antibody, Hyb 1.45 antibody, Hyb 1.46 antibody, Hyb 1.48 antibody, Hyb 1.49 antibody, Hyb 1.51 antibody, Hyb 6.4.1 antibody, F3 antibody, Humanized F3 antibody, C1 antibody, Humanized C1 antibody, 6.4 antibody, anti-mPDGF-C goat IgG antibody, C3.1 antibody, PDGFR-B1 monoclonal antibody, PDGFR-B2 monoclonal antibody, 6D11 monoclonal antibody, Sis 1 monoclonal antibody, PR7212 monoclonal antibody, PR292 monoclonal antibody, HYB 9610 monoclonal antibody, HYB 9611 monoclonal antibody, HYB 9612 monoclonal antibody, or HYB 9613 monoclonal antibody, or a pharmaceutically acceptable salt thereof of any of any of the aforementioned.

In a preferred embodiment, one or more ligand binding molecules described herein are administered in combination with a PDGFR-beta antibody (such as that being developed by Regeneron Inc. for ocular indications) or an anti-PDGF aptamer (such as E10030 being developed by Ophthotech Inc. for ocular indications).

Antibody fragments, for example of a VEGF-A and PGDF inhibitor product, including Fab, Fab′, F(ab′)2, Fv, scFv, are also contemplated. The term “specific for,” when used to describe antibodies of the invention, indicates that the variable regions of the antibodies of the invention recognize and bind the polypeptide of interest exclusively (i.e., able to distinguish the polypeptides of interest from other known polypeptides of the same family, by virtue of measurable differences in binding affinity, despite the possible existence of localized sequence identity, homology, or similarity between family members). It will be understood that specific antibodies may also interact with other proteins (for example, S. aureus protein A or other antibodies in ELISA techniques) through interactions with sequences outside the variable region of the antibodies, and, in particular, in the constant region of the molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, NY (1988), Chapter 6. Antibodies of the invention can be produced using any method well known and routinely practiced in the art.

In another embodiment, methods described herein optionally comprise administering an anti-sense (e.g. antisense to VEGFR-2) nucleic acid molecule to the subject. Antisense nucleic acid molecules to a particular protein (e.g. VEGFR-2) are useful therapeutically to inhibit the translation of mRNAs encoding that protein (e.g. VEGFR-2) where the therapeutic objective involves a desire to eliminate the presence of the protein or to downregulate its levels. VEGFR-2 anti-sense RNA, for example, could be useful as a VEGFR-2 antagonizing agent in the treatment of diseases in which VEGFR-2 is involved as a causative agent, e.g. inflammatory diseases.

An antisense nucleic acid comprises a nucleotide sequence that is complementary to a “sense” nucleic acid encoding a protein (e.g., complementary to the coding strand of a double-stranded cDNA molecule or complementary to an mRNA sequence). (See, e.g., the VEGFR-3 cDNA sequence of SEQ ID NO: 1). Methods for designing and optimizing antisense nucleotides are described in Lima et al., (J Biol Chem;272:626-38. 1997) and Kurreck et al., (Nucleic Acids Res.; 30:1911-8. 2002). In specific aspects, antisense nucleic acid molecules are provided that comprise a sequence complementary to at least about 10, 25, 50, 100, 250 or 500 nucleotides or an entire protein (e.g. VEGFR-2) coding strand, or to only a portion thereof. Nucleic acid molecules encoding fragments, homologs, derivatives and analogs of a protein (e.g. VEGFR-2) or antisense nucleic acids complementary to a protein (VEGFR-2) nucleic acid sequence are also contemplated.

In one embodiment, an antisense nucleic acid molecule is antisense to a “coding region” of the coding strand of a nucleotide sequence encoding a protein such as, e.g. VEGFR-2. The term “coding region” refers to the region of the nucleotide sequence comprising codons which are translated into amino acid residues. In another embodiment, the antisense nucleic acid molecule is antisense to a “conceding region” of the coding strand of a nucleotide sequence encoding the protein such as, e.g.VEGFR-2. The term “conceding region” refers to 5′ and 3′ sequences which flank the coding region that are not translated into amino acids (i.e., also referred to as 5′ and 3′ untranslated regions).

Antisense nucleic acids of the invention can be designed according to the rules of Watson and Crick or Hoogsteen base pairing. The antisense nucleic acid molecule can be complementary to the entire coding region of protein mRNA, but more preferably is an oligonucleotide that is antisense to only a portion of the coding or noncoding region of protein mRNA. An antisense oligonucleotide can be, for example, about 5, 10, 15, 20, 25, 30, 35, 40, 45, or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation.

The antisense nucleic acid molecules are typically administered to a subject or generated in situ such that they hybridize with or bind to cellular mRNA and/or genomic DNA encoding a protein (e.g. VEGFR-2) to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix.

In still another embodiment, protein RNA can be used for induction of RNA interference (RNAi), using double stranded (dsRNA) (Fire et al., Nature 391: 806-811. 1998) or short-interfering RNA (siRNA) sequences (Yu et al., Proc Natl Acad Sci U S A. 99:6047-52, 2002). “RNAi” is the process by which dsRNA induces homology-dependent degradation of complimentary mRNA. In one embodiment, a nucleic acid molecule of the invention is hybridized by complementary base pairing with a “sense” ribonucleic acid of the invention to form the double stranded RNA. The dsRNA antisense and sense nucleic acid molecules are provided that correspond to at least about 20, 25, 50, 100, 250 or 500 nucleotides or an entire protein (e.g. VEGFR-2) coding strand, or to only a portion thereof. In an alternative embodiment, the siRNAs are 30 nucleotides or less in length, and more preferably 21-to 23-nucleotides, with characteristic 2-to 3-nucleotide 3′-overhanging ends, which are generated by ribonuclease III cleavage from longer dsRNAs. See e.g. Tuschl T. (Nat Biotechnol. 20:446-48. 2002). Preparation and use of RNAi compounds is described in U.S. Patent Publication No. 2004/0023390, the disclosure of which is incorporated herein by reference in its entirety. Intracellular transcription of small RNA molecules can be achieved by cloning the siRNA templates into RNA polymerase III (Pol III) transcription units, which normally encode the small nuclear RNA (snRNA) U6 or the human RNAse P RNA H1. Two approaches can be used to express siRNAs: in one embodiment, sense and antisense strands constituting the siRNA duplex are transcribed by individual promoters (Lee, et al. Nat. Biotechnol. 20, 500-505. 2002); in an alternative embodiment, siRNAs are expressed as stem-loop hairpin RNA structures that give rise to siRNAs after intracellular processing (Brummelkamp et al. Science 296:550-553. 2002) (incorporated herein by reference).

The dsRNA/siRNA is most commonly administered by annealing sense and antisense RNA strands in vitro before delivery to the organism. In an alternate embodiment, RNAi may be carried out by administering sense and antisense nucleic acids of the invention in the same solution without annealing prior to administration, and may even be performed by administering the nucleic acids in separate vehicles within a very close timeframe. Nucleic acid molecules encoding fragments, homologs, derivatives and analogs of a protein (such as, e.g. VEGFR-2) or antisense nucleic acids complementary to a mVEGFR-2 nucleic acid sequence are also contemplated.

Aptamers are another nucleic acid based method for interfering with the interaction of receptor and its cognate ligand, such as, e.g. a VEGFR-2 with VEGF-A and a PDGFR with PGDF. Aptamers are DNA or RNA molecules that have been selected from random pools based on their ability to bind other molecules. Aptamers have been selected which bind nucleic acid, proteins, small organic compounds, and even entire organisms. Methods and compositions for identifying and making aptamers are known to those of skill in the art and are described e.g., in U.S. Pat. Nos. 5,840,867 and 5,582,981 each incorporated herein by reference in their entireties.

Recent advances in the field of combinatorial sciences have identified short polymer sequences with high affinity and specificity to a given target. For example, SELEX technology has been used to identify DNA and RNA aptamers with binding properties that rival mammalian antibodies, the field of immunology has generated and isolated antibodies or antibody fragments which bind to a myriad of compounds and phage display has been utilized to discover new peptide sequences with very favorable binding properties. Based on the success of these molecular evolution techniques, it is certain that molecules can be created which bind to any target molecule. A loop structure is often involved with providing the desired binding attributes as in the case of: aptamers which often utilize hairpin loops created from short regions without complimentary base pairing, naturally derived antibodies that utilize combinatorial arrangement of looped hyper-variable regions and new phage display libraries utilizing cyclic peptides that have shown improved results when compared to linear peptide phage display results. Thus, sufficient evidence has been generated to suggest that high affinity ligands can be created and identified by combinatorial molecular evolution techniques. For the present invention, molecular evolution techniques can be used to isolate ligand binding molecules specific for ligands described herein. For more on aptamers, See generally, Gold, L., Singer, B., He, Y. Y., Brody. E., “Aptamers As Therapeutic And Diagnostic Agents,” J. Biotechnol. 74:5-13 (2000). Relevant techniques for generating aptamers may be found in U.S. Pat. No. 6,699,843, which is incorporated by reference in its entirety.

In some embodiments, the aptamer may be generated by preparing a library of nucleic acids; contacting the library of nucleic acids with a growth factor, wherein nucleic acids having greater binding affinity for the growth factor (relative to other library nucleic acids) are selected and amplified to yield a mixture of nucleic acids enriched for nucleic acids with relatively higher affinity and specificity for binding to the growth factor. The processes may be repeated, and the selected nucleic acids mutated and re-screened, whereby a growth factor aptamer is be identified.

In yet another variation, the VEGF-A inhibitor product comprises a soluble ECD fragment of VEGFR-1 that binds VEGF and inhibits VEGF binding to VEGFR-2. cDNA and amino acid sequences of VEGFR-1 are set forth in SEQ ID NOs: 10 and 11. Exemplary ECD fragments of VEGFR-1 are described in U.S. Patent Publication No. 2006/0030000 and International Patent Publication No. WO 2005/087808, the disclosures of which are incorporated herein by reference in their entireties.

### Anti-inflammatory Agents

In another embodiment, the methods described herein optionally comprise administering one or more anti-inflammatory agents to the subject. In some embodiments, the anti-inflammatory agent and the ligand binding molecule are co-administered in a single composition. In other embodiments, the anti-inflammatory agent is administered as a separate composition from the ligand binding molecule. Combinations involving a ligand binding molecule, a VEGF-A inhibitor product, and an anti-inflammatory agent are specifically contemplated. As used herein, the term “anti-inflammatory agent” refers generally to any agent that reduces inflammation or swelling in a subject. A number of exemplary anti-inflammatory agents are recited herein, but it will be appreciated that there may be additional suitable anti-inflammatory agents not specifically recited herein, but which are encompassed by the present invention.

In one variation, the anti-inflammatory agent is a non-steroidal anti-inflammatory drug (NSAID). Exaemplary NSAIDs include, but are not limited to: aspirin, Sulfasalazine™, Asacol™, Dipendtum™, Pentasa™, Anaprox™, Anaprox DS™ (naproxen sodium); Ansaid™ (flurbiprofen); Arthrotec™ (diclofenac sodium+misoprostil); Cataflam™/Voltaren™ (diclofenac potassium); Clinoril™ (sulindac); Daypro™ (oxaprozin); Disalcid™ (salsalate); Dolobid™ (diflunisal); EC Naprosyn™ (naproxen sodium); Feldene™ (piroxicam); Indocin™, Indocin SR™ (indomethacin); Lodine™, Lodine XL™ (etodolac); Motrin™ (ibuprofen); Naprelan™ (naproxen); Naprosyn™ (naproxen); Orudis™, (ketoprofen); Oruvail™ (ketoprofen); Relafen™ (nabumetone); Tolectin™, (tolmetin sodium); Trilisate™ (choline magnesium trisalicylate); Cox-1 inhibitors; Cox-2 Inhibitors such as Vioxx™ (rofecoxib); Arcoxia™ (etoricoxib), Celebrex™ (celecoxib); Mobic™ (meloxicam); Bextra™ (valdecoxib), Dynastat™ paracoxib sodium; Prexige™ (lumiracoxib), and nambumetone. Additional suitable NSAIDs, include, but are not limited to, the following: 5-aminosalicyclic acid (5-ASA, mesalamine, lesalazine), ε-acetamidocaproic acid, S-adenosylmethionine, 3-amino-4-hydroxybutyric acid, amixetrine, anitrazafen, antrafenine, bendazac, bendazac lysinate, benzydamine, beprozin, broperamole, bucolome, bufezolac, ciproquazone, cloximate, dazidamine, deboxamet, detomidine, difenpiramide, difenpyramide, difisalamine, ditazol, emorfazone, fanetizole mesylate, fenflumizole, floctafenine, flumizole, flunixin, fluproquazone, fopirtoline, fosfosal, guaimesal, guaiazolene, isonixirn, lefetamine HCl, leflunomide, lofemizole, lotifazole, lysin clonixinate, meseclazone, nabumetone, nictindole, nimesulide, orgotein, orpanoxin, oxaceprolm, oxapadol, paranyline, perisoxal, perisoxal citrate, pifoxime, piproxen, pirazolac, pirfenidone, proquazone, proxazole, thielavin B, tiflamizole, timegadine, tolectin, tolpadol, tryptamid and those designated by company code number such as 480156S, AA861, AD1590, AFP802, AFP860, AI77B, AP504, AU8001, BPPC, BW540C, CHINOIN 127, CN100, EB382, EL508, F1044, FK-506, GV3658, ITF182, KCNTEI6090, KME4, LA2851, MR714, MR897, MY309, ONO3144, PR823, PV102, PV108, R830, RS2131, SCR152, SH440, SIR133, SPAS510, SQ27239, ST281, SY6001, TA60, TAI-901 (4-benzoyl-1-indancarboxylic acid), TVX2706, U60257, UR2301 and WY41770.

In another variation, the anti-inflammatory agent comprises be a compound that inhibits the interaction of inflammatory cytokines with their receptors. Examples of cytokine inhibitors useful in combination with the specific binding agents of the invention include, for example, antagonists (such as antibodies) of TGF-α (e.g., Remicade), as well as antagonists (such as antibodies) directed against interleukins involved in inflammation. Such interleukins are described herein and preferably include, but are not limited to, IL-1, IL-2, IL-3, IL-4, IL-5, IL-6, IL-8, IL-9, IL-12, IL-13, IL-17, and IL-18. See Feghali, et al., Frontiers in Biosci., 2:12-26 (1997).

In another variation, the anti-inflammatory agent is a corticosteroid. Exemplary corticosteroids include, but are not limited to, difloroasone diacetate, clobetasol propionate, halobetasol propionate, betamethasone, betamethasone dipropionate, cortisone, dexamethasone, fluocinonide, halcinonide desoximethasone, triamcinolone, fluticasone propionate, fluocinolone acetonide, flurandrenolide, mometasone furoate, betamethosone, fluticasone propionate, fluocinolone acetonide, aclometasome dipropionate, methylprednisolone, prednisolone, prednisone, triamicinolone, desonide and hydrocortisone. In another variation, the anti-inflammatory agent is cyclosporine.

### Antibiotics

In another embodiment, the methods described herein optionally further comprise administering an antibiotic to the subject. In some embodiments, the antibiotic and the ligand binding molecule are co-administered in a single composition. In other embodiments, the antibiotic is administered as a separate composition from the ligand binding molecule. Exemplary antibiotics include, but are not limited to, tetracycline, aminoglycosides, penicillins, cephalosporins, sulfonamide drugs, chloramphenicol sodium succinate, erythromycin, vancomycin, lincomycin, clindamycin, nystatin, amphotericin B, amantidine, idoxuridine, p-amino salicyclic acid, isoniazid, rifampin, antinomycin D, mithramycin, daunomycin, adriamycin, bleomycin, vinblastine, vincristine, procarbazine, and imidazole carboxamide.

### Tyrosine Kinase Inhibitors

In another embodiment, the methods described herein optionally further comprise administering a tyrosine kinase inhibitor that inhibits VEGFR-2 and/or VEGFR-3 activity. Exemplary tyrosine kinase inhibitors for use in the methods described herein include, but are not limited to, AEE788 (TKI, VEGFR-2, EGFR: Novartis); ZD6474 (TKI, VEGFR-1,-2,-3, EGFR: Zactima: AstraZeneca); AZD2171 (TKI, VEGFR-1,-2: AstraZeneca); SU 11248 (TKI, VEGFR-1,-2, PDGFR: Sunitinib: Pfizer); AG13925 (TKI, VEGFR-1,-2: Pfizer); AG013736 (TKI, VEGFR-1,-2: Pfizer); CEP-7055 (TKI, VEGFR-1,-2,-3: Cephalon); CP-547,632 (TKI, VEGFR-1,-2: Pfizer); GW7S6024 (TKL VEGFR-1,-2,-3: GlaxoSmithKline); GW786034 (TKI, VEGFR-1,-2,-3: GlaxoSmithKline); sorafenib (TKI, Bay 43-9006, VEGFR-1,-2, PDGFR: Bayer/Onyx); SU4312 (TKI, VEGFR-2, PDGFR: Pfizer); AMG706 (TKI, VEGFR-1,-2,-3: Amgen); XL647 (TKI, EGFR, HER2, VEGFR, ErbB4: Exelixis); XL999 (TKI, FGFR, VEGFR, PDGFR, FII-3: Exelixis); PKC412 (TKI, KIT, PDGFR, PKC, FLT3, VEGFR-2: Novartis); AEE788 (TKI, EGFR, VEGFR2, VEGFR-1: Novartis): OSI-030 (TKI, c-kil, VEGFR: OSI Pharmaceuticals); OS1-817 (TKI c-kit, VEGFR: OSI Pharmaceuticals); DMPQ (TKI, ERGF, PDGFR, ErbB2. p56. pkA, pkC); MLN518 (TKI, Flt3, PDGFR, c-KIT (T53518: Millennium Pharmaceuticals); lestaurinib (TKI, FLT3, CEP-701, Cephalon); ZD 1839 (TKI, EGFR: gefitinib, Iressa: AstraZeneca); OSI-774 (TKI, EGFR: Erlotininb: Tarceva: OSI Pharmaceuticals); lapatinib (TKI, ErbB-2, EGFR, and GD-2016: Tykerb: GlaxoSmithKline). In some embodiments, the methods described herein further comprise administering a tyrosine kinase inhibitor that inhibits angiogenesis to the subject. Exemplary anti-angiogenic tyrosine kinase inhibits and their targets are provided below in Table 2.

TABLE 2
Antiangiogenic tyrosine kinase receptor inhibitors and their targets

| | | | | | | Other |
|---|---|---|---|---|---|---|
| Agent | VEGFR-1 | VEGFR-2 | VEGFR-3 | PDGFR | EGFR | Targets |
| Vandetanib | | • | | | • | RET |
| Sunitinib | • | • | • | • | | KIT, |
| | | | | | | FLT3, |
| | | | | | | RET |
| Axitinib | • | • | • | | | |
| Sorafenib | • | • | • | • | | KIT, |
| | | | | | | RAF, |
| | | | | | | FLT3 |
| Vatalanib | • | • | • | • | | KIT |
| Cediranib | • | • | • | • | | KIT |
| Motesanib | • | • | • | • | | KIT, |
| | | | | | | RET |
| Pazopanib | • | • | • | • | | KIT |
| BIBF 1120 | | • | | • | | FGFR |

Abbreviations:
FGFR, fibroblast-like growth factor receptor;
FLT3, FMS-like tyrosine kinase 3;
KIT, stem cell factor receptor;
RET, glial cell line-derived neurotrophic factor receptor;
VEGFR, vascular endothelial growth factor receptor.

The ligand binding molecules may be administered in combination with more than one additional active compounds or therapies. In one embodiment, a ligand binding molecule of the present invention is administered in combination with a PDGF inhibitor product and a VEGF-A inhibitor product. For example, a ligand binding molecule (such as that comprising the amino acid sequence of SEQ ID NO: 3) may be administered in combination with (i) Aflibercept (Eylea®) and (ii) a PDGFR antibody (such as that being developed by Regeneron Inc. for ocular indications) or a PDGF aptamer (such as E10030 (Fovista™) being developed by Ophthotech Inc. for ocular indications).

### Administration of the Combination Therapy

Combination therapy with one or more of the additional active agents described herein may be achieved by administering to a subject a single composition or pharmacological formulation that includes the ligand binding molecule and the one or more additional active agents, or by administering to the subject two (or more) distinct compositions or formulations, at the same time, wherein one composition includes a ligand binding molecule and the other includes an additional active agent.

Alternatively, the combination therapy employing a ligand binding molecule described herein may precede or follow the second agent treatment by intervals ranging from minutes to weeks. In embodiments where the second agent and the ligand binding molecule are administered separately, one would generally ensure that a significant period of time did not expire between the times of each delivery, such that the agent and the ligand binding molecule would still be able to exert an advantageously combined effect. In such instances, it is contemplated that one would administer both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. Repeated treatments with one or both agents is specifically contemplated.

### Formulations and Pharmaceutically Acceptable Carriers

The present invention also provides pharmaceutical compositions comprising a ligand binding molecule of the invention. Such compositions comprise a therapeutically effective amount of one or more ligand binding molecules and a pharmaceutically acceptable carrier. In one embodiment, such compositions comprise one or more ligand binding molecules and optionally, one or more additional active agents (in the case of a combination therapy). In one embodiment, such compositions comprise one or more ligand binding molecules and optionally one or more additional active agents selected from a PDGF inhibitor product and a VEGF-A inhibitor product. In another embodiment, a composition comprising one or more ligand binding molecules of the invention and another composition comprising a PDGF inhibitor product or a VEGF-A inhibitor product are administered.

The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents.

The compositions may be in the form of solutions, suspensions, emulsion, tablets, pills, capsules, powders, granulates, gels including hydrogels, pastes, ointments, creams, delivery devices, sustained-release formulations, suppositories, injectables, implants, sprays, drops, aerosols and the like. Compositions comprising a ligand binding molecule, one or more additional active agents, or both, can be formulated according to conventional pharmaceutical practice (see, e.g., Remington: The Science and Practice of Pharmacy, (20th ed.) ed. A. R. Gennaro, 2000, Lippincott Williams & Wilkins, Philadelphia, Pa. and Encyclopedia of Pharmaceutical Technology, eds., J. Swarbrick and J. C. Boylan, 1988-2002 Marchcel Dekker, New York). Examples of suitable pharmaceutical carriers are described in “Remington's Pharmaceutical Sciences” by E.W. Martin.

Administration of compositions may be by any suitable means that results in an amount of ligand binding molecule and/or additional active agents that is effective for the treatment or prevention of the particular disease or disorder. Each ligand binding molecule, for example, can be admixed with a suitable carrier substance, and is generally present in an amount of 1-95% by weight of the total weight of the composition. The composition may be provided in a dosage form that is suitable for ophthalmic, oral, parenteral (e.g., intravenous, intramuscular, subcutaneous), rectal, transdermal, nasal, or inhalant administration. In one embodiment, the composition is in a form that is suitable for injection directly in the eye

The ligand binding molecules of the invention, and, where present in combination therapies, the one or more additional active agents, can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Ligand binding molecules and additional active agents of the present invention can possess a sufficiently basic functional group which can react with any of a number of inorganic and organic acids, to form a pharmaceutically acceptable salt. A pharmaceutically-acceptable acid addition salt is formed from a pharmaceutically-acceptable acid, as is well known in the art. Such salts include the pharmaceutically acceptable salts listed in Journal of Pharmaceutical Science, 66, 2-19 (1977) and The Handbook of Pharmaceutical Salts; Properties, Selection, and Use. P. H. Stahl and C. G. Wermuth (ED.s), Verlag, Zurich (Switzerland) 2002, which are hereby incorporated by reference in their entirety.

Pharmaceutically acceptable salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, pheny lacetate, trifluoroacetate, aery late, chloro benzoate, dinitrobenzoate, hydroxybenzoate, methoxybenzoate, methylbenzoate, o-acetoxybenzoate, naphthalene-2-benzoate, isobutyrate, phenylbutyrate, .alpha.-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of a ligand binding molecule and additional active agent having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-, bis-, or tris-(2-hydroxyethyl)amine, 2-hydroxy-tert-butylamine, or tris(hydroxymethyl)methylamine, N,N-di-lower alkyl-N(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like. The term “pharmaceutically acceptable salt” also includes a hydrate of a compound of the invention.

The compositions are, in one useful aspect, administered parenterally (e.g., by intramuscular, intraperitoneal, intravenous, intraocular, intravitreal, retro-bulbar, subconjunctival, subtenon or subcutaneous injection or implant) or systemically. Formulations for parenteral or systemic administration include sterile aqueous or non-aqueous solutions, suspensions, or emulsions. A variety of aqueous carriers can be used, e.g., water, buffered water, saline, and the like. Examples of other suitable vehicles include polypropylene glycol, polyethylene glycol, vegetable oils, gelatin, hydrogels, hydrogenated naphalenes, and injectable organic esters, such as ethyl oleate. Such formulations may also contain auxiliary substances, such as preserving, wetting, buffering, emulsifying, and/or dispersing agents. Biocompatible, biodegradable lactide polymer, lactide/glycolide copolymer, or polyoxyethylene-polyoxypropylene copolymers may be used to control the release of the active ingredients. Alternatively, the compositions can be administered by oral ingestion. Compositions intended for oral use can be prepared in solid or liquid forms, according to any method known to the art for the manufacture of pharmaceutical compositions.

Solid dosage forms for oral administration include capsules, tablets, pills, powders, and granules. Generally, these pharmaceutical preparations contain active ingredients admixed with non-toxic pharmaceutically acceptable excipients. These include, for example, inert diluents, such as calcium carbonate, sodium carbonate, lactose, sucrose, glucose, mannitol, cellulose, starch, calcium phosphate, sodium phosphate, kaolin and the like. Binding agents, buffering agents, and/or lubricating agents (e.g., magnesium stearate) may also be used. Tablets and pills can additionally be prepared with enteric coatings. The compositions may optionally contain sweetening, flavoring, coloring, perfuming, and preserving agents in order to provide a more palatable preparation.

Solid dosage forms can be useful for treatment of ocular disorders. Compositions useful for ocular use include tablets comprising one or more ligand binding molecules in admixture with a pharmaceutically acceptable excipient. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc). Compositions of the present invention may be administered intraocularly by intravitreal injection into the eye as well as by subconjunctival and subtenon injections. Other routes of administration include transcleral, retrobulbar, intraperitoneal, intramuscular, and intravenous. Alternatively, compositions can be administered using a drug delivery device or an intraocular implant.

Liquid dosage forms for oral administration can include pharmaceutically acceptable emulsions, solutions, suspensions, syrups, and soft gelatin capsules. These forms can contain inert diluents commonly used in the art, such as water or an oil medium, and can also include adjuvants, such as wetting agents, emulsifying agents, and suspending agents.

In some instances, the compositions can also be administered topically, for example, by patch or by direct application to a region, such as the epidermis or the eye, susceptible to or affected by a neovascular disorder, or by iontophoresis.

In the case of combination therapies of the present invention, the ligand binding molecules and one or more additional active agents may be admixed in a tablet or other vehicle, or may be partitioned. In one example, the ligand binding molecule is contained on the inside of the tablet, and an additional active agent is on the outside, such that a substantial portion of the additional active agent is released prior to the release of the contained ligand binding molecule.

In one embodiment, compositions that comprise a ligand binding molecule (and optionally one or more additional active agents) can comprise one or more pharmaceutically acceptable excipients. In one embodiment, such excipients include, but are not limited to, buffering agents, non-ionic surfactants, preservatives, tonicity agents, amino acids, sugars and pH-adjusting agents. Suitable buffering agents include, but are not limited to, monobasic sodium phosphate, dibasic sodium phosphate, and sodium acetate. Suitable non-ionic surfactants include, but are not limited to, polyoxyethylene sorbitan fatty acid esters such as polysorbate 20 and polysorbate 80. Suitable preservatives include, but are not limited to, benzyl alcohol. Suitable tonicity agents include, but are not limited to sodium chloride, mannitol, and sorbitol.

Suitable sugars include, but are not limited to, α,α-trehalose dehydrate. Suitable amino acids include, but are not limited to glycine and histidine. Suitable pH-adjusting agents include, but are not limited to, hydrochloric acid, acetic acid, and sodium hydroxide. In one embodiment, the pH-adjusting agent or agents are present in an amount effective to provide a pH of about 3 to about 8, about 4 to about 7, about 5 to about 6, about 6 to about 7, or about 7 to about 7.5. In one embodiment, a composition comprising a ligand binding molecule does not comprise a preservative. In another embodiment, a composition comprising a ligand binding molecule does not comprise an antimicrobial agent. In another embodiment, a composition comprising a ligand binding molecule does not comprise a bacteriostat.

In one embodiment, a composition comprising a ligand binding molecule (and optionally one or more additional active agents) is in the form of an aqueous solution that is suitable for injection. In one embodiment, a composition comprises a ligand binding molecule, a buffering agent, a pH-adjusting agent, and water for injection. In another embodiment, a composition comprises a ligand binding molecule, monobasic sodium phosphate, dibasic sodium phosphate, sodium chloride, hydrochloride acid, and sodium hydroxide. In another embodiment, a composition comprises a ligand binding molecule, phosphate (e.g. monobasic sodium phosphate), trehalose, sodium chloride and polysorbate.

Aqueous compositions useful for practicing the methods of the invention in an ocular setting have ophthalmically compatible pH and osmolality. One or more ophthalmically acceptable pH adjusting agents and/or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, and sodium lactate; and buffers such as citrate/dextrose, sodium bicarbonate and ammonium chloride. Such acids, bases, and buffers are included in an amount required to maintain pH of the composition in an ophthalmically acceptable range. One or more ophthalmically acceptable salts can be included in the composition in an amount sufficient to bring osmolality of the composition into an ophthalmically acceptable range. Such salts include those having sodium, potassium or ammonium cations and chloride, citrate, ascorbate, borate, phosphate, bicarbonate, sulfate, thiosulfate or bisulfite anions.

In some embodiments, the composition comprising a ligand binding molecule of the present invention is formulated for delivery to the eye of a subject. Suitable ophthalmic carriers are known to those skilled in the art and all such conventional carriers may be employed in the present invention. Exemplary compounds incorporated to facilitate and expedite transdermal delivery of topical compositions into ocular or adnexal tissues include, but are not limited to, alcohol (ethanol, propanol, and nonanol), fatty alcohol (lauryl alcohol), fatty acid (valeric acid, caproic acid and capric acid), fatty acid ester (isopropyl myristate and isopropyl n-hexanoate), alkyl ester (ethyl acetate and butyl acetate), polyol (propylene glycol, propanedione and hexanetriol), sulfoxide (dimethylsulfoxide and decylmethylsulfoxide), amide (urea, dimethylacetamide and pyrrolidone derivatives), surfactant (sodium lauryl sulfate, cetyltrimethylannmonium bromide, polaxamers, spans, tweens, bile salts and lecithin), terpene (d-limonene, alphaterpeneol, 1,8-cineole and menthone), and alkanone (N-heptane and N-nonane). Moreover, topically-administered compositions comprise surface adhesion molecule modulating agents including, but not limited to, a cadherin antagonist, a selectin antagonist, and an integrin antagonist. Thus, a particular carrier may take the form of a sterile, ophthalmic ointment, cream, gel, solution, or dispersion. Also including as suitable ophthalmic carriers are slow release polymers, e.g., “Ocusert” polymers, “Hydron” polymers, etc.

Exemplary ophthalmic viscosity enhancers that can be used in the present formulation include: carboxymethyl cellulose sodium; methylcellulose; hydroxypropyl cellulose; hydroxypropylmethyl cellulose; hydroxyethyl cellulose; polyethylene glycol 300; polyethylene glycol 400; polyvinyl alcohol; and providone.

Some natural products, such as veegum, alginates, xanthan gum, gelatin, acacia and tragacanth, may also be used to increase the viscosity of ophthalmic solutions.

A tonicity is important because hypotonic eye drops cause an edema of the cornea, and hypertonic eye drops cause deformation of the cornea. The ideal tonicity is approximately 300 mOsM. The tonicity can be achieved by methods described in Remington: The Science and Practice of Pharmacy, known to those versed in the art.

Stabilizers may also be used such as, for example, chelating agents, e.g., EDTA. Antioxidants may also be used, e.g., sodium bisulfite, sodium thiosulfite, 8-hydroxy quinoline or ascorbic acid. Sterility typically will be maintained by conventional ophthalmic preservatives, e.g., chiorbutanol, benzalkonium chloride, cetylpyridium chloride, phenyl mercuric salts, thimerosal, etc., for aqueous formulations, and used in amounts which are nontoxic and which generally vary from about 0.001 to about 0.1% by weight of the aqueous solution. Conventional preservatives for ointments include methyl and propyl parabens. Typical ointment bases include white petrolatum and mineral oil or liquid petrolatum. However, preserved aqueous carriers are preferred. Solutions may be manually delivered to the eye in suitable dosage form, e.g., eye drops, or delivered by suitable microdrop or spray apparatus typically affording a metered dose of medicament. Examples of suitable ophthalmic carriers include sterile, substantially isotonic, aqueous solutions containing minor amounts, i.e., less than about 5% by weight hydroxypropylmethylcellulose, polyvinyl alcohol, carboxymethylcellulose, hydroxyethylcelullose, glycerine and EDTA. The solutions are preferably maintained at substantially neutral pH and isotonic with appropriate amounts of conventional buffers, e.g., phosphate, borate, acetate, tris.

In some embodiments, penetration enhancers are added to the ophthalmologic carrier. The amount of the ligand binding molecule that will be effective for its intended therapeutic use can be determined by standard clinical techniques based on the present description. In addition, in vitro assays may optionally be employed to help identify optimal dosage ranges. The amount of ligand binding molecule that is admixed with the carrier materials to produce a single dosage can vary depending upon the mammal being treated and the particular mode of administration.

The dosage of the ligand binding molecule can depend on several factors including the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used. Furthermore, the exact individual dosages can be adjusted somewhat depending on a variety of factors, including the specific combination therapies being administered, the time of administration, the route of administration, the nature of the formulation, the rate of excretion, the particular disease being treated (e.g. the particular ocular disorder being treated), the severity of the disorder, and the anatomical location of the neovascular disorder. Some variations in the dosage can be expected.

Generally, when orally administered to a mammal, the dosage of a ligand binding molecule of the present invention is normally 0.001 mg/kg/day to 100 mg/kg/day, 0.01 mg/kg/day to 50 mg/kg/day, or 0.1 mg/kg/day to 10 mg/kg/day. Generally, when orally administered to a human, the dosage of an antagonist of the present invention is normally 0.001 mg to 300 mg per day, 1 mg to 200 mg per day, or 5 mg to 50 mg per day. Dosages up to 200 mg per day may be necessary.

For administration of an antagonist of the present invention by parenteral injection, the dosage is normally 0.1 mg to 250 mg per day, 1 mg to 20 mg per day, or 3 mg to 5 mg per day. Injections may be given up to four times daily.

Generally, when orally or parenterally administered, the dosage of a ligand binding molecule for use in the present invention is normally 0.1 mg to 1500 mg per day, or 0.5 mg to 10 mg per day, or 0.5 mg to 5 mg per day. A dosage of up to 3000 mg per day can be administered. When ophthalmologically administered to a human, for example intravitreally, the dosage of a ligand binding molecule per eye per administration is normally in a range from 0.003 mg, 0.03 mg, 0.03 mg, 0.1 mg or 0.5 mg to 5.0 mg, 4 mg, 3 mg, 2 mg or 1 mg, or 0.5 mg to 1.0 mg. Dosage of a ligand binding molecule is normally in the range 0.003 mg to 5.0 mg per eye per administration, or 0.03 mg to 4.0 mg per eye per administration, or 0.1 mg to 4.0 mg per eye per administration, or 0.03 mg to 3.0 mg per eye per administration, or 0.1 mg to 3.0 mg per eye per administration, or 0.1 mg to 1.0 mg per eye per administration, or 0.5 mg to 4.0 mg per eye per administration, or 0.5 mg to 3.0 mg per eye per administration, 0.5 mg to 2.0 mg per eye per administration, or 1.0 mg to 4.0 mg per eye per administration, or 1.0 mg to 3.0 mg per eye per administration, or 1.0 mg to 2.0 mg per eye per administration. In some embodiments, the ligand binding molecule is administered in a concentration of about 1 mg, or about 2 mg, or about 3 mg, or about 4 mg, or about 5 mg, or about 6 mg per administration per eye. The ligand binding molecule, in some embodiments, is present in any of the concentrations listed above in a volume of 10 μl, 15 μl, 20 μl, 25 μl, 30 μl, 35 μl, 40 μl, 45 μl, 50 μl, 60 μl, 70 μl, 80 μl, 90 μl, 95 μl or 100 μl. In some embodiments, the ligand binding molecule is administered at a concentration of about 2-4 mg/50 μl. The dosage volume can range from 0.01 mL to 0.2 mL administered per eye, or 0.03 mL to 0.15 mL administered per eye, or 0.05 mL to 0.10 mL administered per eye.

In some embodiments, when being administered by intravitreal injection, the ligand binding molecule is administered in a concentration of about 2 mg to about 4 mg per eye (or about 1 mg to about 3 mg, or about 1 mg to about 4 mg, or about 3 mg to about 4 mg, or about 1 mg to about 2 mg per eye). In some embodiments, the ligand binding molecule is administered in a concentration of about 1 mg, or about 2 mg, or about 3 mg, or about 4 mg, or about 5 mg, or about 6 mg per eye. The ligand binding molecule, in some embodiments, is present in any of the concentrations listed above in a volume of 10 μl, 15 μl, 20 μl, 25 μl, 30 μl, 35 μl, 40 μl, 45 μl, 50 μl, 60 μl, 70 μl, 80 μl, 90 μl, 95 μl or 100 μl. In some embodiments, the ligand binding molecule is administered at a concentration of about 2-4 mg/50 μl.

Generally, suitable dosage ranges for intravenous administration are generally about 50-5000 micrograms of active compound per kilogram body weight. Suitable dosage ranges for intranasal administration are generally about 0.01 pg/kg body weight to 1 mg/kg body weight. Effective doses may be extrapolated from dose response curves derived from in vitro or animal model test systems.

For systemic administration, a therapeutically effective dose can be estimated initially from in vitro assays. For example, a dose can be formulated in animal models to achieve a circulating concentration range that includes the IC50 as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. In cases of local administration or selective uptake, the effective local concentration of the compounds may not be related to plasma concentration. One having skill in the art will be able to optimize therapeutically effective local dosages without undue experimentation.

The amount of compound administered will, of course, be dependent on the subject being treated, on the subject's weight, the severity of the affliction, the manner of administration, and the judgment of the prescribing physician. The therapy may be repeated intermittently while symptoms are detectable or even when they are not detectable. The therapy may be provided alone or in combination with other drugs.

Administration of the ligand binding molecule and, when present in combination therapies, an additional agent, can, independently, be one to four times daily or one to four times per month or one to six times per year or once every two, three, four or five years. Administration can be for the duration of one day or one month, two months, three months, six months, one year, two years, three years, and may even be for the life of the patient. In one embodiment, the administration is performed once a month for three months. Chronic, long term administration will be indicated in many cases. The dosage may be administered as a single dose or divided into multiple doses. In general, the desired dosage should be administered at set intervals for a prolonged period, usually at least over several weeks or months, although longer periods of administration of several months or years or more may be needed.

In addition to treating pre-existing disorders, the compositions can be administered prophylactically in order to prevent or slow the onset of these disorders. In prophylactic applications, the composition can be administered to a patient susceptible to or otherwise at risk of a particular disorder, such as an ocular disorder.

### Routes of Administration

The composition containing the ligand binding molecule described herein can be administered to a patient by a variety of means depending, in part, on the type of agent to be administered and the history, risk factors and symptoms of the patient. Routes of administration suitable for the methods of the invention include both systemic and local administration. As used herein, the term “systemic administration” means a mode of administration resulting in delivery of a pharmaceutical composition to essentially the whole body of the patient. Exemplary modes of systemic administration include, without limitation, intravenous injection and oral administration. The term “local administration,” as used herein, means a mode of administration resulting in significantly more pharmaceutical composition being delivered to and about the eyes (or tumor or other target tissue) than to regions distal from the eyes (or tumor or other target tissue).

Systemic and local routes of administration useful in the methods of the invention encompass, without limitation, oral gavage; intravenous injection; intraperitoneal injection; intramuscular injection; subcutaneous injection; transdermal diffusion and electrophoresis; topical eye drops and ointments; periocular and intraocular injection including subconjunctival injection; extended release delivery devices including locally implanted extended release devices; and intraocular and periocular implants including bioerodible and reservoir-based implants.

Thus, in one aspect, a method of treating an ocular disorder associated with retinal neovascularization is practiced by local administration of the ligand binding molecule to the subject. For example, in some embodiments, a pharmaceutical composition comprising the ligand binding molecule is administered topically, or by local injection (e.g., by intraocular, e.g. intravitreal, injection), or is released from an intraocular or periocular implant such as a bioerodible or reservoir-based implant. The composition is preferably administered in an amount effective to inhibit VEGF-C and/or VEGF-D in the eye of the subject from binding to or stimulating VEGFR-2 and/or VEGFR-3 expressed in cells of the eye or vessels of the eye. In the case of combination therapies, the administration of the ligand binding molecule and the additional agent can be sequential in time or concurrent. When administered sequentially, the administration of each can be by the same or different route. In one embodiment, an additional agent (e.g. a VEGF-A or PDGF inhibitor product) is administered within 90 days, 30 days, 10 days, 5 days, 24 hours, 1 hour, 30 minutes, 10 minutes, 5 minutes or one minute of administration of a ligand binding molecule. Where the additional agent is administered prior to the ligand binding molecule, the ligand binding molecule is administered within a time and in an amount such that the total amount of additional agent and ligand binding molecule is effective to treat or prevent the targeted indication, e.g. ocular disorder. Where the ligand binding molecule is administered prior to the additional agent, the additional agent is administered within a time and in an amount such that the total amount of additional agent and ligand binding molecule is effective to treat or prevent the targeted indication, e.g. ocular disorder.

Pharmaceutical compositions according to the invention may be formulated to release the ligand binding molecule and optionally the additional agent in a combination therapy substantially immediately upon administration or at any predetermined time period after administration, using controlled release formulations. For example, a pharmaceutical composition can be provided in sustained-release form. The use of immediate or sustained release compositions depends on the nature of the condition being treated. If the condition consists of an acute disorder, treatment with an immediate release form can be utilized over a prolonged release composition. For certain preventative or long-term treatments, a sustained released composition can also be appropriate.

Administration of the ligand binding molecule or both the ligand binding molecule and one or more additional agents in controlled release formulations can be useful where the ligand binding molecule, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD50) to median effective dose (ED 5 0)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release in which the rate of release outweighs the rate of degradation or metabolism of the active components. For example, controlled release can be obtained by the appropriate selection of formulation parameters and ingredients, including, e.g., appropriate controlled release compositions and coatings. Examples include single or multiple unit tablet or capsule compositions, oil solutions, suspensions, emulsions, microcapsules, microspheres, nanoparticles, patches, and liposomes. Methods for preparing such sustained or controlled release formulations are well known in the art.

The ligand binding molecule and, if present, an additional agent, can also be delivered using a drug-delivery device such as an implant. As used herein, the term “implant” refers to any material that does not significantly migrate from the insertion site following implantation. An implant can be biodegradable, non-biodegradable, or composed of both biodegradable and non-biodegradable materials. A non-biodegradable implant can include, if desired, a refillable reservoir. Implants useful in the methods of the invention include, for example, patches, particles, sheets, plaques, microcapsules and the like, and can be of any shape and size compatible with the selected site of insertion, which can be, without limitation, the posterior chamber, anterior chamber, suprachoroid or subconjunctiva. It is understood that an implant useful in the invention generally releases the implanted pharmaceutical composition at an effective dosage to the eye of the patient over an extended period of time.

A variety of ocular implants and extended release formulations suitable for ocular release are well known in the art, as described, for example, in U.S. Pat. Nos. 5,869,079 and 5,443,505, the disclosures of which are incorporated herein by reference in their entireties. Ocular drug delivery devices can be inserted into a chamber of the eye, such as the anterior or posterior chamber or can be implanted in or on the sclera, choroidal space, or an avascularized region exterior to the vitreous. In one embodiment, the implant can be positioned over an avascular region, such as on the sclera, so as to allow for transcleral diffusion of the ligand binding molecules and any additional agents to the desired site of treatment, e.g., the intraocular space and macula of the eye. Furthermore, the site of transcleral diffusion can be proximal to a site of neovascularization such as a site proximal to the macula. Suitable drug delivery devices are described, for example, in U.S. Publication Nos. 2008/0286334; 2008/0145406; 2007/0184089; 2006/0233860; 2005/0244500; 2005/0244471; and 2005/0244462, and U.S. Pat. Nos. 6,808,719 and 5,322,691, the contents of each of which is herein incorporated by reference in its entirety.

In other embodiments, a ligand binding molecule described herein is applied to the eye via liposomes. In still another embodiment, the ligand binding molecule is contained within a continuous or selective-release device, for example, membranes such as, but not limited to, those employed in the Ocusert™System (Alza Corp., Palo Alto, Calif.). As an additional embodiment, the ligand binding molecule is contained within, carried by, or attached to contact lenses which are placed on the eye. In yet another embodiment, the ligand binding molecule is contained within a swab or sponge which can be applied to the ocular surface. Another embodiment of the present invention involves the ligand binding molecule contained within a liquid spray which can be applied to the ocular surface.

In one embodiment, the implant comprises a ligand binding molecule and optionally, if present, an additional agent, dispersed in a biodegradable polymer matrix. The matrix can comprise PLGA (polylactic acid-polyglycolic acid copolymer), an ester-end capped polymer, an acid end-capped polymer, or a mixture thereof. In another embodiment, the implant comprises a ligand binding molecule and optionally, if present, an additional agent, a surfactant and a lipophilic compound. The lipophilic compound can be present in an amount of about 80-99% by weight of the implant. Suitable lipophilic compounds include, but are not limited to, glyceryl palmitostearate, diethylene glycol monostearate, propylene glycol monostearate, glyceryl monostearate, glyceryl monolinoleate, glyceryl monooleate, glyceryl monopalmitate, glyceryl monolaurate, glyceryl dilaurate, glyceryl monomyristate, glyceryl dimyristate, glyceryl monopalmitate, glyceryl dipalmitate, glyceryl mono stearate, glyceryl distearate, glyceryl monooleate, glyceryl dioleate, glyceryl monolinoleate, glyceryl dilinoleate, glyceryl monoarachidate, glyceryl diarachidate, glyceryl monobehenate, glyceryl dibehenate, and mixtures thereof. In another embodiment, the implant comprises a ligand binding molecule and optionally, if present, an additional agent, housed within a hollow sleeve. The ligand binding molecule and optionally, if present, an additional agent, are delivered to the eye by inserting the sleeve into the eye, releasing the implant from the sleeve into the eye, and then removing the sleeve from the eye. An example of this delivery device is described in U.S. Publication No. 2005/0244462, which is hereby incorporated by reference in its entirety.

In one embodiment, the implant is a flexible ocular insert device adapted for the controlled sustained release of a ligand binding molecule and optionally, if present, an additional agent, into the eye. In one embodiment, the device includes an elongated body of a polymeric material in the form of a rod or tube containing a ligand binding molecule and optionally, if present, an additional agent, and with at least two anchoring protrusions extending radially outwardly from the body. The device may have a length of at least 8 mm and the diameter of its body portion including the protrusions does not exceed 1.9 mm. The sustained release mechanism can, for example, be by diffusion or by osmosis or bioerosion. The insert device can be inserted into the upper or lower formix of the eye so as to be independent of movement of the eye by virtue of the formix anatomy. The protrusions can be of various shapes such as, for example, ribs, screw threads, dimples or bumps, truncated cone-shaped segments or winding braid segments. In a further embodiment, the polymeric material for the body is selected as one which swells in a liquid environment. Thus a device of smaller initial size can be employed. The insert device can be of a size and configuration such that, upon insertion into the upper or lower formix, the device remains out of the field of vision so as to be well retained in place and imperceptible by a recipient over a prolonged period of use. The device can be retained in the upper or lower formix for 7 to 14 days or longer. An example of this device is described in U.S. Pat. No. 5,322,691, which is hereby incorporated by reference in its entirety.

In another aspect, a method of inhibiting neovascularization in a subject who has been diagnosed with a tumor practiced by local administration of the ligand binding molecule to the subject. For example, in some embodiments, a pharmaceutical compositions comprising the ligand binding molecule is administered locally to the tumor or to the organ or tissue from which the tumor has been surgically removed. In such embodiments, the composition is preferably administered in an amount effective to inhibit neovascularization in the tumor.

In instances where the ligand binding molecule is a nucleic acid molecule, administration of a pharmaceutical composition containing the nucleic acid molecule can be carried out using one of numerous methods well known in the art of gene therapy. Such methods include, but are not limited to, lentiviral transformation, adenoviral transformation, cytomegaloviral transformation, microinjection and electroporation.

### Kits And Unit Doses

The invention also relates to kits comprising one or more pharmaceutical compositions and instructions for use. A ligand binding molecule may be packaged or formulated together with another ligand binding molecule or other therapeutic described herein, e.g., in a kit or package or unit dose, to permit co-administration; these two components may be formulated together (i.e. in admixture) or in separate compositions (i.e.not in admixture) and in individual dosage amounts. Each of the kits' compositions can be contained in a container. In some embodiments, the two components to the kit/unit dose are packaged with instructions for administering the two compounds to a human subject for treatment of one of the disorders and diseases described herein.

The kits can comprise a container. The container can be used to separate components and include, for example, a divided bottle or a divided foil packet. The separate compositions may also, if desired, be contained within a single, undivided container. The kits can also comprise directions for the administration of the components. The kits are particularly advantageous when the separate components are administered in different dosage forms, are administered at different dosage levels, or when titration of the individual antagonists is desired.

All publications and patents mentioned herein are hereby incorporated by reference in their entirety as if each individual publication or patent was specifically and individually indicated to be incorporated by reference. In case of conflict, the present application, including any definitions herein, will control.

### EXAMPLES

### Example 1—ECD Fragments of VEGFR Proteins

Experiments were performed to characterize fragments and variants and fusions of VEGFR-3 and/or VEGFR-2 and/or VEGFR-1 that are effective to bind target ligands, such as VEGF-C and/or VEGF-D and/or VEGF-A. See International Patent Publication Nos: WO 2005/087808, WO 2005/000895, WO 2006/088650, WO 2006/099154, WO 2004/106378, WO 2005/123104 and U.S. Pat. No. 7,855,178, all of which are incorporated herein by reference in their entireties. These studies demonstrate that the ECDs of these receptors can be truncated, and also that domains from different receptors can be recombined, to form ligand binding molecules.

### Example 2—Generation of VGX-301-ΔN2 Ligand Binding Molecule

A ligand binding molecule comprising Ig-like domains I-III of VEGFR-3 (referred to herein as “VGX-300”) was prepared as described in Makinen et al., Nat. Med., 7:199-205, 2001, the disclosure of which is incorporated herein by reference in its entirety.

A key feature of the VGX-300 molecule is that it contains 12 glycosylation sites; 2×6 potential N-linked glycosylation sites, 5 on each receptor fragment (VEGFR-3 Ig-like domains I, II, and III) and 1 on each Fc region gamma chain. There is no evidence for O-linked glycosylation. Glycosylation characteristics can have an effect on PK but, Fc glycans have little effect on PK (Jones et al, Glycobiology, 17(5), 2007 pp. 529-540). Briefly, the asialoglycoprotein receptor binds to complex-type N-linked glycan structures in which two or more sialic acids are absent, wherein the underlying galactose (Gal) residues become the terminal saccharides. In addition, the mannose (Man) receptor recognizes high-Man N-linked glycans and terminal N-acetylglucosamine (tGlcNAc) residues. Both of these receptors can cause rapid metabolic clearance of proteins.

In order to identify which glycosylation sites are important for product activity, sequential deletion of each of the five putative N-linked sites was undertaken. Five primer pairs were used to introduce single mutations into the VGX-300 coding region to destroy the consensus attachment for each of the five N-linked glycans (N-Q). The primer pairs used are as follows:

N1 sense:
(SEQ ID NO: 12)
5′ GACCCCCCCGACCTTGCAGATCACGGAGGAGTCACAC 3′
N1 anti-sense:
(SEQ ID NO: 13)
5′ GTGTGACTCCTCCGTGATCTGCAAGGTCGGGGGGGTC 3′
N2 sense:
(SEQ ID NO: 14)
5′ CTGCACGAGGTACATGCCCAGGACACAGGCAGCTACGTC 3′
N2 anti-sense:
(SEQ ID NO: 15)
5′ GACGTAGCTGCCTGTGTCCTGGGCATGTACCTCGTGCAG 3′
N3 sense:
(SEQ ID NO: 16)
5′ GTCCATCCCCGGCCTCCAAGTCACGCTGCGCTCGC 3′
N3 anti-sense:
(SEQ ID NO: 17)
5′ GCGAGCGCAGCGTGACTTGGAGGCCGGGGATGGAC 3′
N4 sense:
(SEQ ID NO: 18)
5′ GGGAGAAGCTGGTCCTCCAGTGCACCGTGTGGGCTGA 3′
N4 anti-sense:
(SEQ ID NO: 19)
5′ TCAGCCCACACGGTGCACTGGAGGACCAGCTTCTCCC 3′
N5 sense:
(SEQ ID NO: 20)
5′ AGCATCCTGACCATCCACCAGGTCAGCCAGCACGACCT 3′
N5 anti-sense:
(SEQ ID NO: 21)
5′ AGGTCGTGCTGGCTGACCTGGTGGATGGTCAGGATGCT 3′

The presence of mutations was confirmed by sequencing, following which the plasmid vectors were transiently transfected into 293T cells (HEK). Culture samples were analyzed by western blot. Viable constructs were then progressed to transient suspension-adapted 293F cells (HEK) and the supernatants purified by ProSepA chromatography and gel filtration, for further testing by enzyme-linked immunosorbent assay (ELISA) and BaF/3 bioassay to determine yield and activity. Table 3 below summarizes expression data and activity of each resulting mutant.

TABLE 3

| | | | Activity | |
|---|---|---|---|---|
| Mutant | Gel Filtration Profile | Yield | ELISA | BaF/3 |
| Parent | Monomer evident, | Fair | Reference | Reference |
| | some aggregate | | | |
| ΔN1 | Monomer evident, | Poor | ≥1 log less | ≥1 log less |
| | substantial aggregate | | than parent | than parent |
| ΔN2 | Monomer evident, | Fair | Comparable | Comparable |
| | some aggregate | | to parent | to parent |
| ΔN3 | Severe aggregation | Poor | ≥1 log less | ≥1 log less |
| | | | than parent | than parent |
| ΔN4 | Some monomer evident, | Poor | ≥1 log less | ≥1 log less |
| | significant aggregate | | than parent | than parent |
| ΔN5 | Some monomer evident, | Poor | ≥1 log less | ≥1 log less |
| | significant aggregate | | than parent | than parent |

Table 3 shows that only the N2 mutant (referred to herein as “VGX-301-ΔN2”) exhibited favorable expression and activity characteristics relative to the parent molecule (i.e., VGX-300). VGX-301-ΔN2 and VGX-300 parent were produced in CHO and HEK cells by transient expression, and the pharmacokinetics (PK) of each molecule was examined as follows. Sprague-Dawley rats were allocated into either groups of 2, 3 or 5 per compound in each experiment. The rats in each group received a single dose of VGX-300 or VGX-301-ΔN2 via intravenous administration as a bolus injection at a dose concentration of 1 mg/kg. Interim blood samples were collected by lateral tail vein puncture on Day-1 (Pre-dose) and a total of 12 time points post-dose, ranging from 5 min to 14-days post-initial treatment. Serum samples were prepared from each blood sample and tested using a quantitative VEGF-C ligand-capture ELISA to determine the circulating concentration of each compound. The results of these analyses were then used for calculation of the pharmacokinetic parameters. PK data of VGX-300 and VGX-301-ΔN2 is provided below in Table 4.

TABLE 4

| | | | | |
|---|---|---|---|---|
| | | | | T_{1/2} |
| | Dose | AUC _{0-last/Dose} | Cₘₐₓ | (el) |
| Compound | (mg/kg) | (hr*ng/mL) | (ng/mL) | (hr) |

Transient CHO Expression (Expt 1)

| | | | | |
|---|---|---|---|---|
| VGX-300 | 1 | 15,115 | 5,702 | 16.9 |
| VGX-301-ΔN2 | 1 | 23,236 | 12,076 | 22.6 |

Transient CHO Expression (Expt 2)

| | | | | |
|---|---|---|---|---|
| VGX-300 | 1 | 35,310 | 16,000 | 46 |
| VGX-301-ΔN2 | 1 | 55,071 | 20,000 | 42 |

Transient HEK Expression

| | | | | |
|---|---|---|---|---|
| VGX-300 | 1 | 18,738 | 8,500 | 6.4 |
| VGX-301-ΔN2 | 1 | 90,750 | 13,250 | 15.3 |

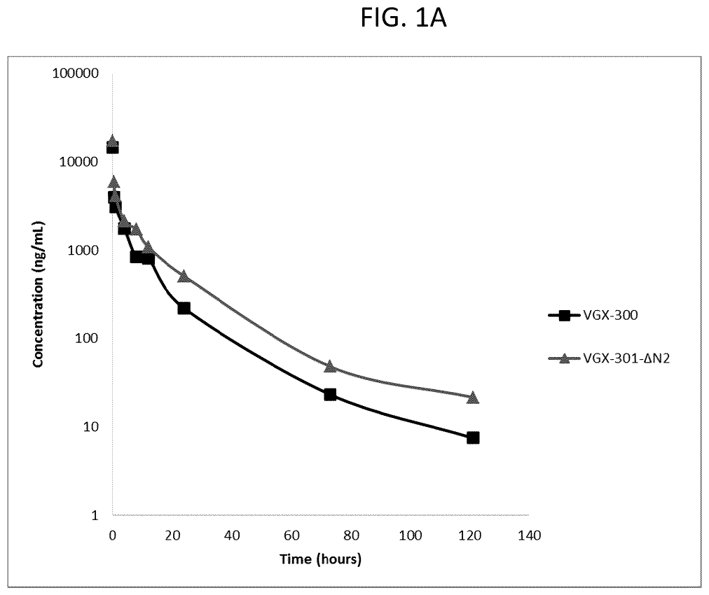

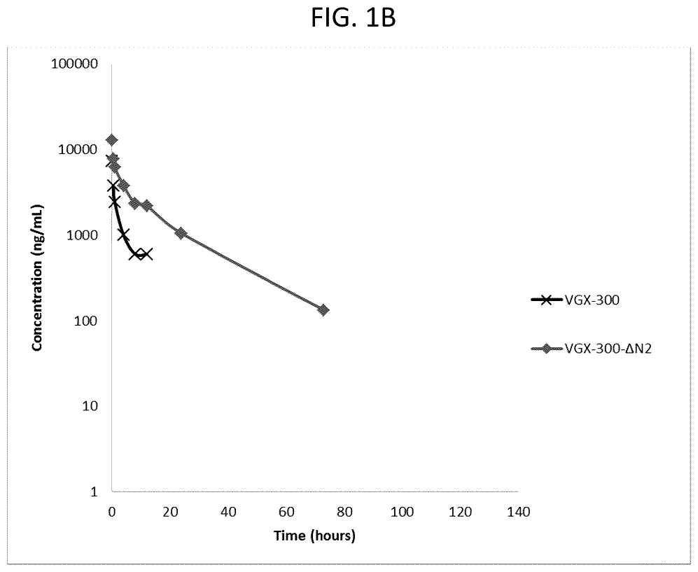

The PK curves provided in FIGS. 1A and 1B and the data from Table 4 show that the VGX-301-ΔN2 may have a beneficial effect on PK by comparison to VGX-300 produced in the same expression system.

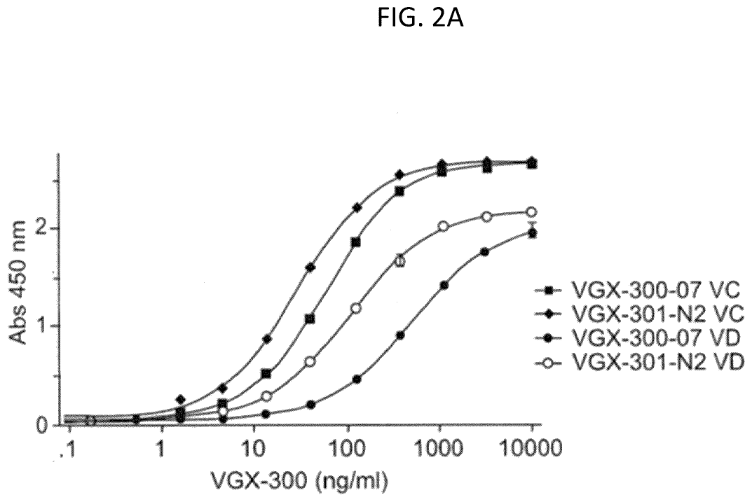

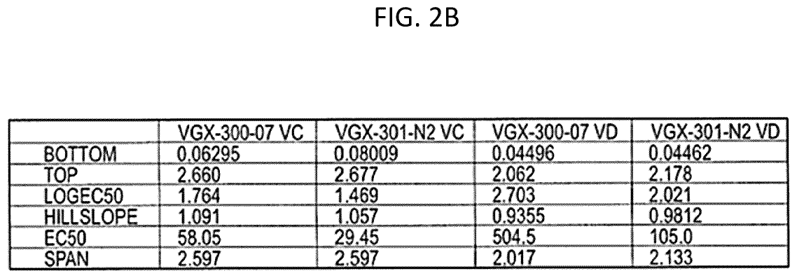

To determine the specificity of VGX-300 and VGX-301-ΔN2 binding to VEGF-C and VEGF-D, VEGF-C or VEGF-D (2 μg/mL) were pre-coated onto ELISA plates and used as capture antigens. Increasing concentrations of either VGX-300 or VGX-301-ΔN2 (0 to 10 μg/mL) were applied to the plate and detected with rabbit anti-human IgG-horseradish peroxidase conjugate using a tetramethylbenzidine substrate kit. Results indicated that both VGX-300 and VGX-301-ΔN2 bound to both VEGF-C and VEGF-D. See FIG. 2. Surprisingly, VGX-301-ΔN2 demonstrated stronger binding to both ligands than VGX-300.

### Example 4—VGX-300 and VGX-301-ΔN2 Binding Affinity

The binding of VEGF-C and VEGF-D to VGX-300 or VGX-301-AN2 was analyzed by surface plasmon resonance (SPR) performed using the ProteOn XPR36 biosensor (Bio-Rad). Either VGX-300 or VGX-301-ΔN2 was captured onto protein G′ immobilized onto a GLM sensor chip and the affinity of the molecule to VEGF-C or VEGF-D was measured. The results of the affinity experiment are provided below in Table 5.

TABLE 5

| | kₐ(M⁻¹s⁻¹) × 10⁶ | k_{d}(s⁻¹) × 10⁻⁵ | K_{D}(pM) |
|---|---|---|---|
| | Human VEGF-C | | |
| VGX-300 | 2.18 ± 0.05 | 1.11 ± 0.12 | 5.1 ± 0.6 |
| VGX-301-ΔN2 | 2.79 ± 0.04 | 1.03 ± 0.08 | 3.7 ± 0.3 |
| | Human VEGF-D | | |
| VGX-300 | 4.9 ± 0.1 | 3.23 ± 0.16 | 625 ± 21 |
| VGX-301-ΔN2 | 5.7 ± 0.1 | 3.88 ± 0.03 | 677 ± 12 |

The data presented in Table 5 above shows that the VGX-300 and VGX-301-ΔN2 samples bound human VEGF-C and VEGF-D with near identical affinities, with both molecules showing stronger binding to VEGF-C than VEGF-D.

### Example 5-VGX-301-ΔN2 Blocks VEGF-C and VEGF-D Binding and Cross-Linking of VEGFR-3

Cell-based assays have been developed to evaluate the capacity of VEGF family ligands to bind and cross-link VEGFR-2 and VEGFR-3. These bioassays have been employed to study the neutralizing activity of VGX-300 and VGX-301-AN2. The bioassay cell lines consist of the mouse IL-3 dependent pro-B cell line Ba/F3, stably transfected with a chimeric receptor consisting of the ECD of VEGFR-2 or VEGFR-3, fused in-frame to the transmembrane and intracellular domains of the mouse erythropoietin receptor (as described in Example 5 of WO 2005/087808, the disclosure of which is incorporated herein by reference in its entirety). In the absence of IL-3, these cells survive and proliferate only in the presence of growth factors capable of binding and cross-linking the ECD of the respective VEGFR.

Briefly, Ba/F3 cells transfected with VEGFR-2 or VEGFR-3 (10,000 cells/well; 96 well plate) were cultured in media supplemented with VEGF-C or VEGF-D in the presence of increasing concentrations of VGX-300 of VGX-301-ΔN2(0-100 μg/mL) for 48 hours at 37° C. Cell proliferation was measured using WST 1 reagent; cells were incubated for 4 hours at 37° C. with WST-1 and the absorbance measured at 450 nm (n=3; error bars=standard error of the mean, SEM).

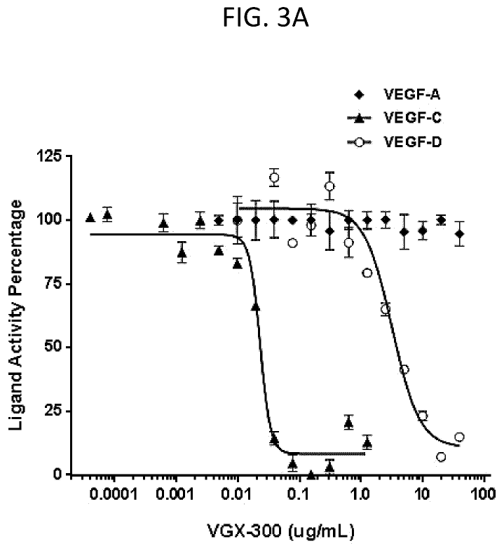

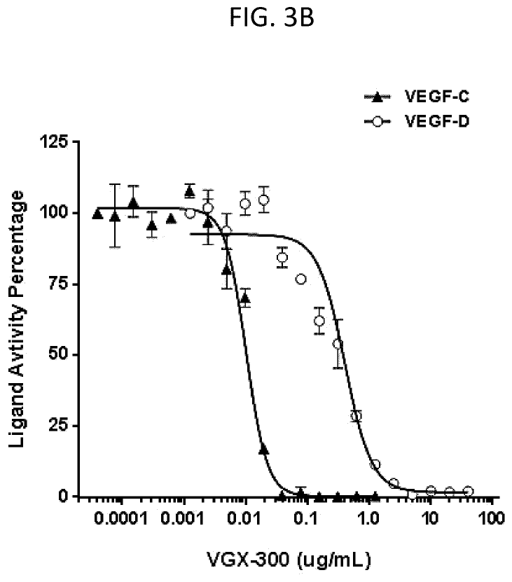

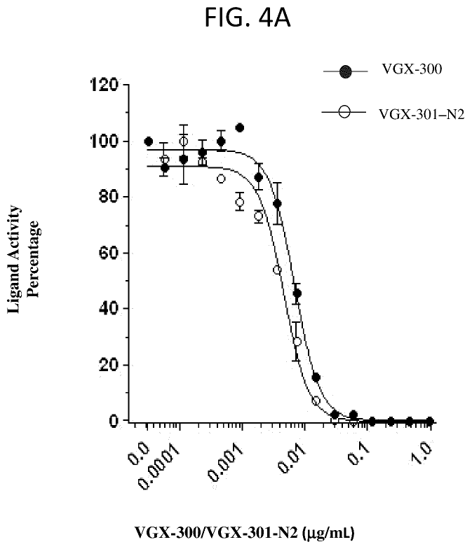

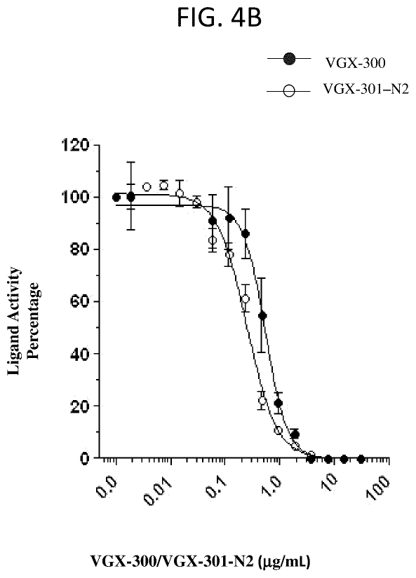

Results indicated that VGX-300 neutralized the activity of VEGF-C and VEGF-D, as demonstrated by the dose-responsive inhibition of VEGF-C and VEGF-D in the VEGFR-2 and VEGFR-3 Ba/F3 bioassays. VGX-300 showed enhanced potency in neutralizing VEGF-C compared to VEGF-D in both the VEGFR-2 and-3 assays. See FIGS. 3A-3B and 4A-4B. Analysis of VGX-301-ΔN2 demonstrated that this molecule was also able to block both VEGF-C and VEGF-D from binding to VEGFR-3. The neutralizing activity of VGX-301-N2 was slightly stronger than that of VGX-300. See FIGS. 4A-4B. Table 6 shows the binding (IC50) of VGX-300 and VGX-301-ΔN2 to VEGF-C and VEGF-D in the VEGFR-3 Ba/F3 bioassay.

TABLE 6

| | VGX-300 | VGX-301-ΔN2 |
|---|---|---|
| | IC₅₀ | IC₅₀ |
| VEGF-D ligand 300 ng/mL | 544.9 | 251.7 |
| VEGF-C ligand 5 ng/mL | 6.8 | 4.5 |

### Example 6—Ocular Distribution and Pharmacokinetics of VGX-300 and VGX-301ΔN2 Following Intravitreal Administration

This study was conducted to investigate the ocular distribution and pharmacokinetics of VGX-300, VGX-301-ΔN2 and Aflibercept (EYLEA) following a single intravitreal dose to pigmented rabbits.

The study design consisted of 3 groups, 8 female rabbits allocated per group. Animals are administered 500 μg of radiolabelled VGX-300, VGX-301-ΔN2 or Aflibercept via a 50 μL bolus intravitreal injection into both eyes.

| | | | | | Target | |
|---|---|---|---|---|---|---|
| | Number | | | Target | Dose | |
| | of | | Dose | Dose | Volume | Samples |
| Group | Females | Formulation | Route | Level | (μL/eye) | Collected |
| 1 | 8 | ¹²⁵I-VGX-300 | IVT | 500 μg | 50 | Blood and |
| | | | | | | ocular |
| | | | | | | tissues |
| 2 | 8 | ¹²⁵I-Aflibercept | IVT | 500 μg | 50 | Blood and |
| | | (EYLEA) | | | | ocular |
| | | | | | | tissues |
| 3 | 8 | ¹²⁵I-VGX-301- | IVT | 500 μg | 50 | Blood and |
| | | ΔN2 | | | | ocular |
| | | | | | | tissues |

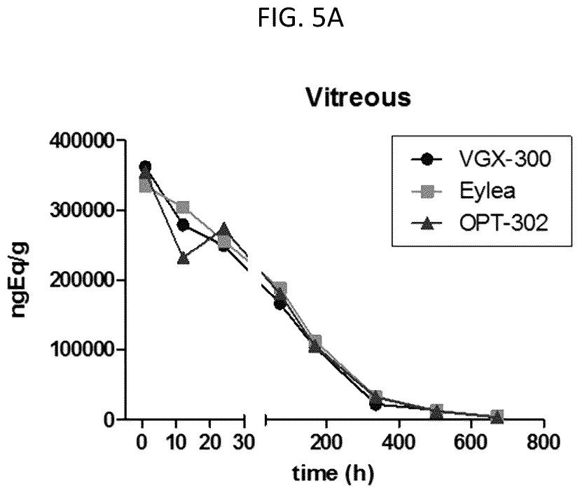

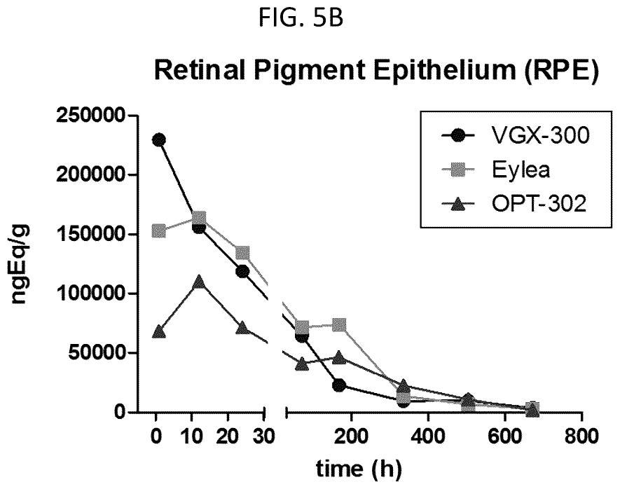

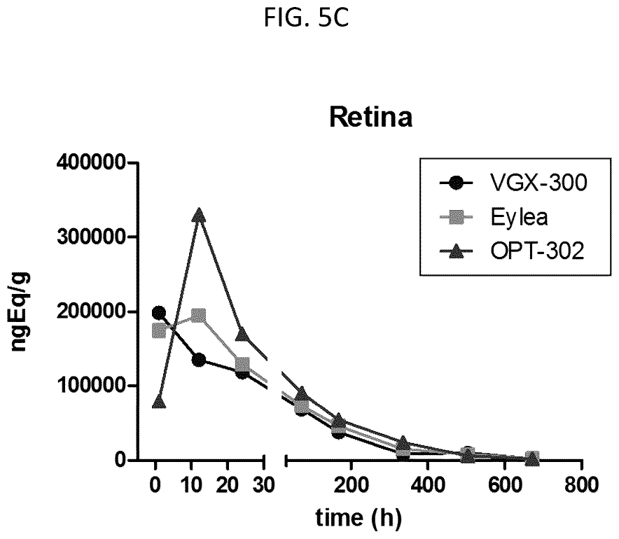

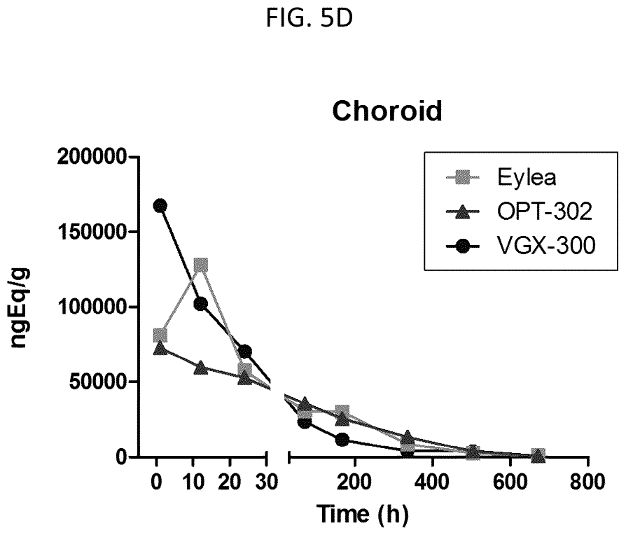

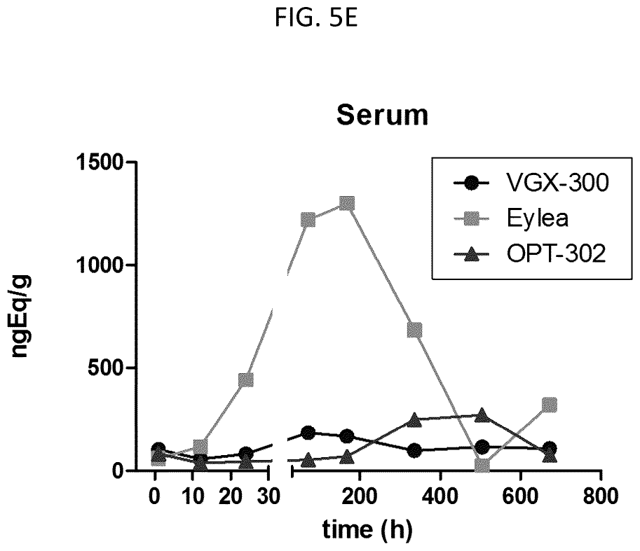

One animal per group was euthanized at 1, 12, 24, 72, 168, 366, 504 and 672 hours following dose administration. Blood, processed to serum, and selected ocular tissues were collected at each time point and the concentration of radioactivity determined by radioanalysis. The ocular tissues collected included the aqueous humor, choroid, cornea, iris-cilary body (ICB), lens, optic nerve, retina, retinal pigmented epithelium (RPE), sclera, trabecular meshwork and vitreous humor. FIGS. 5A-5E show the mean concentrations of radioactivity in various tissues and serum over the time period monitored.

The test articles, ¹²⁵I-VGX-300, ¹²⁵I-Aflibercept (EYLEA) and ¹²⁵I-VGX-301-ΔN2, were well tolerated, stable in the vitreous humor and had prolonged exposure to ocular tissues of both the posterior segment and the anterior segment. Although there were differences in the serum exposure of ¹²⁵I-VGX-300 and ¹²⁵I-VGX-301-ΔN2 following intravitreal administration, both ¹²⁵I-VGX-300 and ¹²⁵I-VGX-301-ΔN2 had only minor systemic exposure compared to that of aflibercept (EYLEA), likely as a result of clearance via absorption into the choroid and also by aqueous humor outflow. The PK and biodistribution of ¹²⁵I-VGX-300 and ¹²⁵I-VGX-301-AN2 observed in this study were similar for both compounds, and comparable to that of ¹²⁵I-Aflibercept (EYLEA).

### Example 7—Retinopathy of Prematurity Model

The following Example is an exemplary assay to evaluate VGX-300 and VGX-301-ΔN2 for their ability to inhibit the onset of retinal neovascularization using the ROP model. In this model, postnatal day 7 (P7) mice are exposed to hyperoxia (75% oxygen) for 5 days (to P12). After hyperoxic exposure, P12 mice are returned to normoxia, and administered an intravitreal injection of human isotype control antibody, VGX-300, VGX-301-ΔN2, Eylea (VEGF-Trap), VGX-300+Eylea or VGX-301-ΔN2+Eylea. All mice are then housed under normoxic conditions for 5 days before sacrifice at P17, enucleation and fixation in 10% formalin/PBS. Vessels will be quantified in each group using H&E and/or IHC staining methods.

### Example 8—Argon Laser-Induced Choroidal Neovascularization (CNV)

In this model of age-related macular degeneration (AMD), CNV is induced by argon laser-induced rupture of Bruch's membrane in mice on Day 0 (3 burns per mouse). Groups of 10 mice are studied and treatment administered via weekly intravitreal injections (at day 0 and day 7) of human isotype control antibody, VGX-301-ΔN2, VGX-300, Eylea (VEGF-Trap), VGX-301-ΔN2+Eylea or VGX-300+Eylea. At day 14, animals are sacrificed and choroidal flat mounts prepared and stained with ICAM-2 to visualize the neovascularisation by fluorescence microscopy.

It is contemplated that VGX-301-ΔN2, as a single-agent, will significantly inhibit choroidal neovascularisation in a mouse model of neovascular AMD, comparable to the effect demonstrated by Eylea®.

### Example 9—Inhibitory Effect of Ligand Binding Molecules on VEGF-C Mediated Tumor Growth and Metastasis

To demonstrate the ability of a ligand binding molecule described herein to inhibit tumor growth and/or metastasis, any accepted tumor model may be employed. Exemplary models include animals predisposed to developing various types of cancers, animals injected with tumors or tumor cells or tumor cell lines from the same or different species, including optionally cells transformed to recombinantly overexpress one or more growth factors such as VEGF-C, or VEGF-D. To provide a model for tumors in vivo in which multiple growth factors are detectable, it is possible to transform tumor cell lines with exogenous DNA to cause expression of multiple growth factors.

A ligand binding molecule described herein may be administered directly, e.g., in protein form by i.v. transfusion or by implanted micropumps, or in nucleic acid form as part of a gene therapy regimen. Subjects are preferably grouped by sex, weight, age, and medical history to help minimize variations amongst subjects.

Efficacy is measured by a decrease in tumor, size (volume) and weight. One may also examine the nature of the effect on tumor size, spreads (metastases) and number of tumors. For example, use of specific cell markers can be used to show the effect on angiogenesis relative to lymphangiogenesis, a VEGF-A binding construct expected to have a greater effect on the former, and a VEGF-C binding construct expected to have a greater effect on the latter. Animals may be looked at as a whole for survival time and changes in weight. Tumors and specimens are examined for evidence of angiogenesis, lymphangiogenesis, and/or necrosis. SCID mice may be used as subjects for the ability of a ligand binding molecule described herein to inhibit or prevent the growth of tumors. The ligand binding molecule used in the therapy is generally chosen such that it binds to a growth factor ligand expressed by the tumor cell, especially growth factors that are overexpressed by the tumor cell relative to non-neoplastic cells in the subject. In the SCID model, tumor cells, e.g., MCF-7 cells, may be transfected with a virus encoding a particular growth factor under the control of a promoter or other expression control sequence that provides for overexpression of the growth factor as described in WO 02/060950. Alternatively, other cell lines may be employed, e.g., HT-1080, as described in U.S. Pat. No. 6,375,929. One may transfect the tumor cells with as many growth factor ligands as one desires to overexpress, or a tumor cell line may be chosen that already overexpresses one or more growth factor ligands of interest. One group of subjects is implanted with cells that have been mock-transfected, i.e., with a vector lacking a growth factor ligand insert.

Either before, concurrently with, or after the tumor implantation of the above-described cells, subjects are treated with a particular ligand binding molecule. There are a number of different ways of administering the ligand binding molecule. In vivo and/or ex vivo gene therapy may be employed. For example, cells may be transfected with a adenovirus, or other vector, that encodes the ligand binding molecule and implanted with the tumor cells expressing the growth factor(s), the cells transfected with the ligand binding molecule may be the same as those transformed with growth factor(s) (or already overexpressing the growth factor(s)). In some embodiments, an adenovirus that encodes that ligand binding molecule is injected in vivo, e.g., intravenously. In some embodiments, the ligand binding molecule itself (e.g., in protein form) is administered either systematically or locally, e.g., using a micropump. When testing the efficacy of a particular binding construct, at least one control is normally employed. For example, in the case of a vector-based therapy, a vector with an empty insert or LacZ is employed, or the insert may be a ligand binding molecule comprising a complete ECD of VEGFR-3 capable of binding VEGF-C or VEGF-D, such a control may employ more than one ECD construct if necessary (e.g., for binding multiple ligands if binding constructs with multiple ligand binding affinities are employed).

### A. Exemplary Procedures

### Preparation Of Plasmid Expression Vectors, Tranfection of Cells, and Testing of the Same

A cDNA encoding VEGF-C or VEGF-D or combinations thereof are introduced into a pEBS7 plasmid (Peterson and Legerski, Gene, 107: 279-84, 1991.). This same vector may be used for the expression of the ligand binding molecule.

The MCF-7S1 subclone of the human MCF-7 breast carcinoma cell line is transfected with the plasmid DNA by electroporation and stable cell pools are selected and cultured as previously described (Egeblad and Jaattela, Int. J. Cancer, 86: 617-25, 2000). The cells are metabolically labeled in methionine and cysteine free MEM (Gibco) supplemented with 100 μCi/ml ³⁵S-methionine and ³⁵S-cysteine (Redivue Pro-Mix, Amersham Pharmacia Biotech). The labeled growth factors are immunoprecipitated from the conditioned medium using antibodies against the expressed growth factor(s). The immunocomplexes and the binding complexes are precipitated using protein A sepharose (Amersham Pharmacia Biotech), washed twice in 0.5% BSA, 0.02% Tween 20 in PBS and once in PBS and analyzed in SDS-PAGE under reducing conditions.

### Subject Preparation and Treatment

Cells (20,000/well) are plated in quadruplicate in 24-wells, trypsinized on replicate plates after 1, 4, 6, or 8 days and counted using a hemocytometer. Fresh medium is provided after 4 and 6 days. For the tumorgenesis assay, sub-confluent cultures are harvested by trypsination, washed twice and 10⁷ cells in PBS are inoculated into the fat pads of the second (axillar) mammary gland of ovariectomized SCID mice, carrying subcutaneous 60-day slow-release pellets containing 0.72 mg 17β-estradiol (Innovative Research of America). The ovarectomy and implantation of the pellets are performed 4-8 days before tumor cell inoculation.

The cDNA coding for the binding construct(s) is subcloned into the pAdBgIII plasmid and the adenoviruses produced as previously described (Laitinen et al., Hum. Gene Ther., 9: 1481-6, 1998). The ligand binding molecule(s) or LacZ control (Laitinen et al., Hum. Gene Ther., 9: 1481-6, 1998) adenoviruses, 10⁹ pfu/mouse, are injected intravenously into the SCID mice 3 hours before the tumor cell inoculation.

### Analysis of Treatment Efficacy

Tumor length and width are measured twice weekly in a blinded manner, and the tumor volume are calculated as the length×width×depth×0.5, assuming that the tumor is a hemi-ellipsoid and the depth is the same as the width (Benz et al., Breast Cancer Res. Treat., 24: 85-95, 1993).

The tumors are excised, fixed in 4% paraformaldehyde (pH 7.0) for 24 hours, and embedded in paraffin. Sections (7 μm) are immunostained with monoclonal antibodies against, for example, PECAM-1 (Pharmingen), VEGFR-1, VEGFR-2, VEGFR-3 (Kubo et al., Blood, 96: 546-553, 2000) or PCNA (Zymed Laboratories), PDGFR-α, PDGFR-β or polyclonal antibodies against LYVE-1 (Banerji et al., J Cell Biol, 144: 789-801, 1999), VEGF-C (Joukov et al., EMBO J., 16: 3898-911, 1997), laminin according to published protocols (Partanen et al., Cancer, 86: 2406-12, 1999), or any of the growth factors. The average of the number of the PECAM-1 positive vessels are determined from three areas (60× magnification) of the highest vascular density (vascular hot spots) in a section. All histological analyses are performed using blinded tumor samples.

Three weeks after injection of adenovirus constructs and/or protein therapy, four mice from each group are narcotized, the ventral skin is opened and a few microliters 3% Evan's blue dye (Sigma) in PBS is injected into the tumor. The drainage of the dye from the tumor is followed macroscopically.

Imaging and monitoring of blood and blood proteins to provide indication of the health of subjects and the extent of tumor vasculature may also be performed.

### Example 10—Effects on Tumor Progression in Subjects Using a Combined Therapy of a Ligand Binding Molecule and a Chemotherapeutic Agent

This study is carried out to test the efficacy of using a ligand binding molecule described herein in combination with other anti-cancer therapies. Such therapies include chemotherapy, radiation therapy, anti-sense therapy, RNA interference, and monoclonal antibodies directed to cancer targets. The combinatorial effect may be additive, but it is preferably synergistic in its anti-cancer effects, e.g., prevention, suppression, regression, and elimination of cancers, prolongation of life, and/or reduction in side-effects.

Subjects are divided into groups with one group receiving a chemotherapeutic agent, one group receiving a ligand binding molecule, and one group receiving both a chemotherapeutic agent and a ligand binding molecule at regular periodic intervals, e.g., daily, weekly or monthly. In human studies, the subjects are generally grouped by sex, weight, age, and medical history to help minimize variations among subjects. Ideally, the subjects have been diagnosed with the same type of cancer. In human or non-human subjects, progress can be followed by measuring tumor size, metastases, weight gain/loss, vascularization in tumors, and white blood cells counts.

Biopsies of tumors are taken at regular intervals both before and after beginning treatment. For example, biopsies are taken just prior to treatment, at one week, and then at one month intervals, thereafter, or whenever possible, e.g., as tumors are excised. One examines the biopsies for cell markers, and overall cell and tissue morphology to assess the effectiveness of the treatment. In addition, or in the alternative, imagining techniques may be employed.

For non-human animal studies, an additional placebo control may be employed. Animal studies, performed in accordance with NIH guidelines, also provide the advantage of the insertion of relatively uniform cancer cell population, and tumors that selectively overproduce the one or more growth factors targeted by the ligand binding molecule.

1-62. (canceled)

63. A method of treating a subject having an ocular disorder associated with neovascularization, vascular endothelial cell proliferation, vascular permeability, edema, or inflammation, the method comprising:
(a) administering to the subject a composition comprising:
(i) a soluble, ligand binding molecule comprising a ligand binding polypeptide, wherein the ligand binding polypeptide comprises an amino acid sequence having at least 95% identity to the sequence of amino acids defined by positions 25-210 of SEQ ID NO: 2, with the proviso that positions of the polypeptide corresponding to positions 104-106 of SEQ ID NO: 2 are not identical to N-X-S or N-X-T, and wherein the ligand binding polypeptide and the ligand binding molecule bind to at least one ligand polypeptide selected from human VEGF-C, VEGF-D, and PIGF; and
(ii) a pharmaceutically acceptable diluent, adjuvant, excipient, or carrier; and
(b) administering to the subject a composition comprising a VEGF-A inhibitor product and a pharmaceutically acceptable diluent, adjuvant, excipient, or carrier;
the method comprising administering to the subject the compositions (a) and (b) in amounts effective to inhibit neovascularization, vascular endothelial cell proliferation, vascular permeability, edema, or inflammation in the subject.

64. The method according to claim 63, wherein the ligand binding molecule comprises the soluble, ligand binding polypeptide connected to a heterologous peptide.

65. The method according to claim 64, wherein the heterologous peptide comprises an immunoglobulin constant domain fragment.

66. The method according to claim 65, wherein the ligand binding polypeptide comprises an amino acid sequence having at least 95% identity to the sequence of amino acids defined by positions 25-765 of SEQ ID NO: 2, with the proviso that positions of the polypeptide corresponding to positions 104-106 of SEQ ID NO: 2 are not identical to N-X-S or N-X-T.

67. The method according to claim 65, wherein the ligand binding polypeptide comprises an amino acid sequence having at least 95% identity to the sequence of amino acids defined by positions 25-314 of SEQ ID NO: 2, with the proviso that positions of the polypeptide corresponding to positions 104-106 of SEQ ID NO: 2 are not identical to N-X-S or N-X-T.

68. The method according to claim 67, wherein the ligand binding polypeptide lacks VEGFR-3 Ig-like domains 4-7, lacks a VEGFR-3 transmembrane domain, and lacks a VEGFR-3 intracellular domain.

69. The method according to claim 68, wherein the ligand binding polypeptide retains four N-glycosylation sequon sites corresponding to positions 33-35 of SEQ ID NO: 2, positions 166-168 of SEQ ID NO: 2, positions 251-253 of SEQ ID NO: 2, and positions 299-301 of SEQ ID NO: 2, and wherein the ligand binding polypeptide is glycosylated at said four N-glycosylation sequon sites.

70. The method according to claim 63, wherein the ligand binding molecule comprises the amino acid sequence set forth in SEQ ID NO: 3.

71. The method according to claim 64, wherein the ligand binding polypeptide is connected to the heterologous peptide by amide bonding and forms a single polypeptide chain.

72. The method according to claim 64, wherein the amino acid in the ligand binding polypeptide corresponding to position 104 of SEQ ID NO: 2 is deleted or replaced with another amino acid.

73. The method according to claim 64, wherein the amino acid in the ligand binding polypeptide corresponding to position 104 of SEQ ID NO: 2 is deleted or replaced with another amino acid selected from the group consisting of glutamine, aspartate, glutamate, arginine, and lysine.

74. The method according to claim 64, wherein the compositions are administered locally to the eye of the subject.

75. The method according to claim 64, wherein the compositions are administered by intravitreal injection.

76. The method according to claim 75, wherein the composition comprising the soluble, ligand binding molecule is administered in an amount effective to inhibit VEGF-C and/or VEGF-D in the eye of the subject from binding to or stimulating VEGFR-2 and/or VEGFR-3 expressed in cells of the eye or vessels of the eye.

77. The method according to claim 76, wherein the composition comprising the soluble, ligand binding molecule and the composition comprising the VEGF-A inhibitor product are administered separately.

78. The method according to claim 76, wherein the VEGF-A inhibitor product comprises a VEGF receptor-Fc fusion protein.

79. The method according to claim 76, wherein the VEGF-A inhibitor product comprises aflibercept.

80. The method according to claim 76, wherein the VEGF-A inhibitor product comprises ranibizumab or bevacizumab.

81. The method according to claim 75, wherein the composition that comprises the soluble, ligand binding molecule further comprises the composition that comprises VEGF-A inhibitor product, and (a) and (b) are co-administered.

82. The method according to claim 81, wherein the VEGF-A inhibitor product comprises a VEGF receptor-Fc fusion protein.

83. The method according to claim 81, wherein the VEGF-A inhibitor product comprises aflibercept.

84. The method according to claim 81, wherein the VEGF-A inhibitor product comprises ranibizumab or bevacizumab.

85. The method according to claim 76, wherein the subject has an ocular disorder associated with retinal neovascularization that is hypo-responsive to a VEGF-A inhibitor standard-of-care regimen.


SEQUENCE LISTING (21 sequences)

SEQ ID NO: 1 - DNA, 4195 bp, Homo sapiens
  source 1..4195: mol_type = genomic DNA
  CDS 20..3913
ccacgcgcagcggccggagatgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctgaactgcaccgtgtgggctgagtttaactcaggtgtcacctttgactgggactacccagggaagcaggcagagcggggtaagtgggtgcccgagcgacgctcccagcagacccacacagaactctccagcatcctgaccatccacaacgtcagccagcacgacctgggctcgtatgtgtgcaaggccaacaacggcatccagcgatttcgggagagcaccgaggtcattgtgcatgaaaatcccttcatcagcgtcgagtggctcaaaggacccatcctggaggccacggcaggagacgagctggtgaagctgcccgtgaagctggcagcgtaccccccgcccgagttccagtggtacaaggatggaaaggcactgtccgggcgccacagtccacatgccctggtgctcaaggaggtgacagaggccagcacaggcacctacaccctcgccctgtggaactccgctgctggcctgaggcgcaacatcagcctggagctggtggtgaatgtgcccccccagatacatgagaaggaggcctcctcccccagcatctactcgcgtcacagccgccaggccctcacctgcacggcctacggggtgcccctgcctctcagcatccagtggcactggcggccctggacaccctgcaagatgtttgcccagcgtagtctccggcggcggcagcagcaagacctcatgccacagtgccgtgactggagggcggtgaccacgcaggatgccgtgaaccccatcgagagcctggacacctggaccgagtttgtggagggaaagaataagactgtgagcaagctggtgatccagaatgccaacgtgtctgccatgtacaagtgtgtggtctccaacaaggtgggccaggatgagcggctcatctacttctatgtgaccaccatccccgacggcttcaccatcgaatccaagccatccgaggagctactagagggccagccggtgctcctgagctgccaagccgacagctacaagtacgagcatctgcgctggtaccgcctcaacctgtccacgctgcacgatgcgcacgggaacccgcttctgctcgactgcaagaacgtgcatctgttcgccacccctctggccgccagcctggaggaggtggcacctggggcgcgccacgccacgctcagcctgagtatcccccgcgtcgcgcccgagcacgagggccactatgtgtgcgaagtgcaagaccggcgcagccatgacaagcactgccacaagaagtacctgtcggtgcaggccctggaagcccctcggctcacgcagaacttgaccgacctcctggtgaacgtgagcgactcgctggagatgcagtgcttggtggccggagcgcacgcgcccagcatcgtgtggtacaaagacgagaggctgctggaggaaaagtctggagtcgacttggcggactccaaccagaagctgagcatccagcgcgtgcgcgaggaggatgcgggacgctatctgtgcagcgtgtgcaacgccaagggctgcgtcaactcctccgccagcgtggccgtggaaggctccgaggataagggcagcatggagatcgtgatccttgtcggtaccggcgtcatcgctgtcttcttctgggtcctcctcctcctcatcttctgtaacatgaggaggccggcccacgcagacatcaagacgggctacctgtccatcatcatggaccccggggaggtgcctctggaggagcaatgcgaatacctgtcctacgatgccagccagtgggaattcccccgagagcggctgcacctggggagagtgctcggctacggcgccttcgggaaggtggtggaagcctccgctttcggcatccacaagggcagcagctgtgacaccgtggccgtgaaaatgctgaaagagggcgccacggccagcgagcaccgcgcgctgatgtcggagctcaagatcctcattcacatcggcaaccacctcaacgtggtcaacctcctcggggcgtgcaccaagccgcagggccccctcatggtgatcgtggagttctgcaagtacggcaacctctccaacttcctgcgcgccaagcgggacgccttcagcccctgcgcggagaagtctcccgagcagcgcggacgcttccgcgccatggtggagctcgccaggctggatcggaggcggccggggagcagcgacagggtcctcttcgcgcggttctcgaagaccgagggcggagcgaggcgggcttctccagaccaagaagctgaggacctgtggctgagcccgctgaccatggaagatcttgtctgctacagcttccaggtggccagagggatggagttcctggcttcccgaaagtgcatccacagagacctggctgctcggaacattctgctgtcggaaagcgacgtggtgaagatctgtgactttggccttgcccgggacatctacaaagaccctgactacgtccgcaagggcagtgcccggctgcccctgaagtggatggcccctgaaagcatcttcgacaaggtgtacaccacgcagagtgacgtgtggtcctttggggtgcttctctgggagatcttctctctgggggcctccccgtaccctggggtgcagatcaatgaggagttctgccagcggctgagagacggcacaaggatgagggccccggagctggccactcccgccatacgccgcatcatgctgaactgctggtccggagaccccaaggcgagacctgcattctcggagctggtggagatcctgggggacctgctccagggcaggggcctgcaagaggaagaggaggtctgcatggccccgcgcagctctcagagctcagaagagggcagcttctcgcaggtgtccaccatggccctacacatcgcccaggctgacgctgaggacagcccgccaagcctgcagcgccacagcctggccgccaggtattacaactgggtgtcctttcccgggtgcctggccagaggggctgagacccgtggttcctccaggatgaagacatttgaggaattccccatgaccccaacgacctacaaaggctctgtggacaaccagacagacagtgggatggtgctggcctcggaggagtttgagcagatagagagcaggcatagacaagaaagcggcttcaggtagctgaagcagagagagagaaggcagcatacgtcagcattttcttctctgcacttataagaaagatcaaagactttaagactttcgctatttcttctactgctatctactacaaacttcaaagaggaaccaggaggacaagaggagcatgaaagtggacaaggagtgtgaccactgaagcaccacagggaaggggttaggcctccggatgactgcgggcaggcctggataatatccagcctcccacaagaagctggtggagcagagtgttccctgactcct

SEQ ID NO: 2 - protein, 1298 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFR

SEQ ID NO: 3 - protein, 537 aa, Homo sapiens
YSMTPPTLNITEESHVIDTGDSLSISCRGQHPLEWAWPGAQEAPATGDKDSEDTGVVRDCEGTDARPYCKVLLLHEVHAQDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 10 aa, synthetic construct
  REGION 1..10: note = Synthetic linker
PIEGRGGGGG

SEQ ID NO: 5 - DNA, 2292 bp, Homo sapiens
  source 1..2292: mol_type = genomic DNA
  CDS 1..2292
atggagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaa

SEQ ID NO: 6 - protein, 764 aa, Homo sapiens
MESKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLE

SEQ ID NO: 7 - protein, 13 aa, synthetic construct
  REGION 1..13: note = Synthetic linker
EFGAGLVLGGQFM

SEQ ID NO: 8 - protein, 15 aa, synthetic construct
  REGION 1..15: note = Synthetic linker
GGGGSGGGGSGGGGS

SEQ ID NO: 9 - protein, 16 aa, synthetic construct
  REGION 1..16: note = Synthetic linker
GGSGGSGGGGSGGGGS

SEQ ID NO: 10 - DNA, 5777 bp, Homo sapiens
  misc_feature 1..5777: note = VEGFR-1
  source 1..5777: mol_type = genomic DNA
  CDS 250..4266
gcggacactcctctcggctcctccccggcagcggcggcggctcggagcgggctccggggctcgggtgcagcggccagcgggcctggcggcgaggattacccggggaagtggttgtctcctggctggagccgcgagacgggcgctcagggcgcggggccggcggcggcgaacgagaggacggactctggcggccgggtcgttggccgggggagcgcgggcaccgggcgagcaggccgcgtcgcgctcaccatggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagagatcaggaagcaccatacctcctgcgaaacctcagtgatcacacagtggccatcagcagttccaccactttagactgtcatgctaatggtgtccccgagcctcagatcacttggtttaaaaacaaccacaaaatacaacaagagcctggaattattttaggaccaggaagcagcacgctgtttattgaaagagtcacagaagaggatgaaggtgtctatcactgcaaagccaccaaccagaagggctctgtggaaagttcagcatacctcactgttcaaggaacctcggacaagtctaatctggagctgatcactctaacatgcacctgtgtggctgcgactctcttctggctcctattaaccctccttatccgaaaaatgaaaaggtcttcttctgaaataaagactgactacctatcaattataatggacccagatgaagttcctttggatgagcagtgtgagcggctcccttatgatgccagcaagtgggagtttgcccgggagagacttaaactgggcaaatcacttggaagaggggcttttggaaaagtggttcaagcatcagcatttggcattaagaaatcacctacgtgccggactgtggctgtgaaaatgctgaaagagggggccacggccagcgagtacaaagctctgatgactgagctaaaaatcttgacccacattggccaccatctgaacgtggttaacctgctgggagcctgcaccaagcaaggagggcctctgatggtgattgttgaatactgcaaatatggaaatctctccaactacctcaagagcaaacgtgacttattttttctcaacaaggatgcagcactacacatggagcctaagaaagaaaaaatggagccaggcctggaacaaggcaagaaaccaagactagatagcgtcaccagcagcgaaagctttgcgagctccggctttcaggaagataaaagtctgagtgatgttgaggaagaggaggattctgacggtttctacaaggagcccatcactatggaagatctgatttcttacagttttcaagtggccagaggcatggagttcctgtcttccagaaagtgcattcatcgggacctggcagcgagaaacattcttttatctgagaacaacgtggtgaagatttgtgattttggccttgcccgggatatttataagaaccccgattatgtgagaaaaggagatactcgacttcctctgaaatggatggctcccgaatctatctttgacaaaatctacagcaccaagagcgacgtgtggtcttacggagtattgctgtgggaaatcttctccttaggtgggtctccatacccaggagtacaaatggatgaggacttttgcagtcgcctgagggaaggcatgaggatgagagctcctgagtactctactcctgaaatctatcagatcatgctggactgctggcacagagacccaaaagaaaggccaagatttgcagaacttgtggaaaaactaggtgatttgcttcaagcaaatgtacaacaggatggtaaagactacatcccaatcaatgccatactgacaggaaatagtgggtttacatactcaactcctgccttctctgaggacttcttcaaggaaagtatttcagctccgaagtttaattcaggaagctctgatgatgtcagatatgtaaatgctttcaagttcatgagcctggaaagaatcaaaacctttgaagaacttttaccgaatgccacctccatgtttgatgactaccagggcgacagcagcactctgttggcctctcccatgctgaagcgcttcacctggactgacagcaaacccaaggcctcgctcaagattgacttgagagtaaccagtaaaagtaaggagtcggggctgtctgatgtcagcaggcccagtttctgccattccagctgtgggcacgtcagcgaaggcaagcgcaggttcacctacgaccacgctgagctggaaaggaaaatcgcgtgctgctccccgcccccagactacaactcggtggtcctgtactccaccccacccatctagagtttgacacgaagccttatttctagaagcacatgtgtatttatacccccaggaaactagcttttgccagtattatgcatatataagtttacacctttatctttccatgggagccagctgctttttgtgatttttttaatagtgctttttttttttgactaacaagaatgtaactccagatagagaaatagtgacaagtgaagaacactactgctaaatcctcatgttactcagtgttagagaaatccttcctaaacccaatgacttccctgctccaacccccgccacctcagggcacgcaggaccagtttgattgaggagctgcactgatcacccaatgcatcacgtaccccactgggccagccctgcagcccaaaacccagggcaacaagcccgttagccccaggggatcactggctggcctgagcaacatctcgggagtcctctagcaggcctaagacatgtgaggaggaaaaggaaaaaaagcaaaaagcaagggagaaaagagaaaccgggagaaggcatgagaaagaatttgagacgcaccatgtgggcacggagggggacggggctcagcaatgccatttcagtggcttcccagctctgacccttctacatttgagggcccagccaggagcagatggacagcgatgaggggacattttctggattctgggaggcaagaaaaggacaaatatcttttttggaactaaagcaaattttagacctttacctatggaagtggttctatgtccattctcattcgtggcatgttttgatttgtagcactgagggtggcactcaactctgagcccatacttttggctcctctagtaagatgcactgaaaacttagccagagttaggttgtctccaggccatgatggccttacactgaaaatgtcacattctattttgggtattaatatatagtccagacacttaactcaatttcttggtattattctgttttgcacagttagttgtgaaagaaagctgagaagaatgaaaatgcagtcctgaggagagttttctccatatcaaaacgagggctgatggaggaaaaaggtcaataaggtcaagggaagaccccgtctctataccaaccaaaccaattcaccaacacagttgggacccaaaacacaggaagtcagtcacgtttccttttcatttaatggggattccactatctcacactaatctgaaaggatgtggaagagcattagctggcgcatattaagcactttaagctccttgagtaaaaaggtggtatgtaatttatgcaaggtatttctccagttgggactcaggatattagttaatgagccatcactagaagaaaagcccattttcaactgctttgaaacttgcctggggtctgagcatgatgggaatagggagacagggtaggaaagggcgcctactcttcagggtctaaagatcaagtgggccttggatcgctaagctggctctgtttgatgctatttatgcaagttagggtctatgtattta

SEQ ID NO: 11 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLLIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 12 - DNA, 37 bp, synthetic construct
  misc_feature 1..37: note = Synthetic primer
  source 1..37: mol_type = other DNA
gacccccccgaccttgcagatcacggaggagtcacac

SEQ ID NO: 13 - DNA, 37 bp, synthetic construct
  misc_feature 1..37: note = Synthetic primer
  source 1..37: mol_type = other DNA
gtgtgactcctccgtgatctgcaaggtcgggggggtc

SEQ ID NO: 14 - DNA, 39 bp, synthetic construct
  misc_feature 1..39: note = Synthetic primer
  source 1..39: mol_type = other DNA
ctgcacgaggtacatgcccaggacacaggcagctacgtc

SEQ ID NO: 15 - DNA, 39 bp, synthetic construct
  misc_feature 1..39: note = Synthetic primer
  source 1..39: mol_type = other DNA
gacgtagctgcctgtgtcctgggcatgtacctcgtgcag

SEQ ID NO: 16 - DNA, 35 bp, synthetic construct
  misc_feature 1..35: note = Synthetic primer
  source 1..35: mol_type = other DNA
gtccatccccggcctccaagtcacgctgcgctcgc

SEQ ID NO: 17 - DNA, 35 bp, synthetic construct
  misc_feature 1..35: note = Synthetic primer
  source 1..35: mol_type = other DNA
gcgagcgcagcgtgacttggaggccggggatggac

SEQ ID NO: 18 - DNA, 37 bp, synthetic construct
  misc_feature 1..37: note = Synthetic primer
  source 1..37: mol_type = other DNA
gggagaagctggtcctccagtgcaccgtgtgggctga

SEQ ID NO: 19 - DNA, 37 bp, synthetic construct
  misc_feature 1..37: note = Synthetic primer
  source 1..37: mol_type = other DNA
tcagcccacacggtgcactggaggaccagcttctccc

SEQ ID NO: 20 - DNA, 38 bp, synthetic construct
  misc_feature 1..38: note = Synthetic primer
  source 1..38: mol_type = other DNA
agcatcctgaccatccaccaggtcagccagcacgacct

SEQ ID NO: 21 - DNA, 38 bp, synthetic construct
  misc_feature 1..38: note = Synthetic primer
  source 1..38: mol_type = other DNA
aggtcgtgctggctgacctggtggatggtcaggatgct